SEQUENCE LISTING (57 sequences)

SEQ ID NO: 1 - DNA, 20 bp, Artificial Sequence
  Primer
caacaacccctttgccaaag

SEQ ID NO: 2 - DNA, 20 bp, Artificial Sequence
  Primer
tcccccaagcagttgacagt

SEQ ID NO: 3 - DNA, 19 bp, Artificial Sequence
  Primer
agaaccggccccttatgaa

SEQ ID NO: 4 - DNA, 18 bp, Artificial Sequence
  Primer
agttcgcgcaggatgtcc

SEQ ID NO: 5 - DNA, 21 bp, Artificial Sequence
  Primer
cacccaggaaagacagcaacc

SEQ ID NO: 6 - DNA, 21 bp, Artificial Sequence
  Primer
gcaagagctcttgtccattga

SEQ ID NO: 7 - DNA, 18 bp, Artificial Sequence
  Primer
ccgctgagagggcttcac

SEQ ID NO: 8 - DNA, 22 bp, Artificial Sequence
  Primer
tgcaggagtaggccacattaca

SEQ ID NO: 9 - DNA, 25 bp, Artificial Sequence
  Primer
gatgcattcatgagtattgccaagt

SEQ ID NO: 10 - DNA, 21 bp, Artificial Sequence
  Primer
gtggaccactcggatgagctc

SEQ ID NO: 11 - DNA, 20 bp, Artificial Sequence
  Primer
tcggcattttgaacgagctc

SEQ ID NO: 12 - DNA, 20 bp, Artificial Sequence
  Primer
gaaaagcccgaaagagtctc

SEQ ID NO: 13 - DNA, 20 bp, Artificial Sequence
  Primer
atgacatgaaccgacccttc

SEQ ID NO: 14 - DNA, 20 bp, Artificial Sequence
  Primer
tgtgttggttgtagagggca

SEQ ID NO: 15 - DNA, 15 bp, Artificial Sequence
  Primer
atgcctggctcagac

SEQ ID NO: 16 - DNA, 19 bp, Artificial Sequence
  Primer
gtcctgcattaaggagtcg

SEQ ID NO: 17 - DNA, 22 bp, Artificial Sequence
  Primer
cagcagcgatcatccctcaaag

SEQ ID NO: 18 - DNA, 21 bp, Artificial Sequence
  Primer
caggaccaggatctcttgctg

SEQ ID NO: 19 - DNA, 21 bp, Artificial Sequence
  Primer
gagttcatggtgctgtcttcc

SEQ ID NO: 20 - DNA, 21 bp, Artificial Sequence
  Primer
gagttcatggtgctgtcttcc

SEQ ID NO: 21 - DNA, 20 bp, Artificial Sequence
  Primer
catgcaggaggtggtacctt

SEQ ID NO: 22 - DNA, 20 bp, Artificial Sequence
  Primer
cagacgcaagcatttctcag

SEQ ID NO: 23 - DNA, 21 bp, Artificial Sequence
  Primer
aggtgttggcattctcacaag

SEQ ID NO: 24 - DNA, 23 bp, Artificial Sequence
  Primer
gcttatcatgtttacaggttccc

SEQ ID NO: 25 - DNA, 20 bp, Artificial Sequence
  Primer
tcaggtgcaaggtgaagttg

SEQ ID NO: 26 - DNA, 20 bp, Artificial Sequence
  Primer
ggccactgttaccactgctt

SEQ ID NO: 27 - DNA, 21 bp, Artificial Sequence
  Primer
aaggaagctctcagtcgtcaa

SEQ ID NO: 28 - DNA, 21 bp, Artificial Sequence
  Primer
gagtcaggtttcctggtcgag

SEQ ID NO: 29 - DNA, 26 bp, Artificial Sequence
  Primer
gaactttgtataccatttgtcagacg

SEQ ID NO: 30 - DNA, 20 bp, Artificial Sequence
  Primer
ctgggtggcagtaacaacct

SEQ ID NO: 31 - DNA, 20 bp, Artificial Sequence
  Primer
agtaaccgaggcctgtcctt

SEQ ID NO: 32 - DNA, 19 bp, Artificial Sequence
  Primer
gtcactcggcaactcagga

SEQ ID NO: 33 - DNA, 20 bp, Artificial Sequence
  Primer
catcaccggtcagaaaacaa

SEQ ID NO: 34 - DNA, 20 bp, Artificial Sequence
  Primer
gtcaccatggccaatgtagg

SEQ ID NO: 35 - DNA, 20 bp, Artificial Sequence
  Primer
tcagaacttcagtggctgga

SEQ ID NO: 36 - DNA, 20 bp, Artificial Sequence
  Primer
gaggccaattttgtctccac

SEQ ID NO: 37 - DNA, 20 bp, Artificial Sequence
  Primer
tggtgaaggtcggtgtgaac

SEQ ID NO: 38 - DNA, 24 bp, Artificial Sequence
  Primer
ccatgtagttgaggtcaatgaacg

SEQ ID NO: 39 - DNA, 60 bp, Artificial Sequence
  Primer
aatgatacggcgaccaccgagatctacactatggtaattgtgtgccagcmgccgcggtaa

SEQ ID NO: 40 - DNA, 68 bp, Artificial Sequence
  Primer
caagcagaagacggcatacgagatnnnnnnnnnnnnagtcagtcagccggactachvgggtwtctaat

SEQ ID NO: 41 - DNA, 31 bp, Artificial Sequence
  Primer
tatggtaattgtgtgccagcmgccgcggtaa

SEQ ID NO: 42 - DNA, 32 bp, Artificial Sequence
  Primer
agtcagtcagccggactachvgggtwtctaat

SEQ ID NO: 43 - DNA, 32 bp, Artificial Sequence
  Primer
attagawacccbdgtagtccggctgactgact

SEQ ID NO: 44 - DNA, 24 bp, Artificial Sequence
  Primer
gaaggaagctaactggaaaaactc

SEQ ID NO: 45 - DNA, 22 bp, Artificial Sequence
  Primer
caggcccccagcacaacctact

SEQ ID NO: 46 - DNA, 21 bp, Artificial Sequence
  Primer
tggctggaaagatggagacaa

SEQ ID NO: 47 - DNA, 22 bp, Artificial Sequence
  Primer
aaagctctctcttgcatcactg

SEQ ID NO: 48 - DNA, 23 bp, Artificial Sequence
  Primer
tgccatcattctttgcatcttga

SEQ ID NO: 49 - DNA, 21 bp, Artificial Sequence
  Primer
ccgtgaacttctgaacagcct

SEQ ID NO: 50 - DNA, 20 bp, Artificial Sequence
  Primer
cattcgaacgtctgccctat

SEQ ID NO: 51 - DNA, 20 bp, Artificial Sequence
  Primer
cctccaatggatcctcgtta

SEQ ID NO: 52 - DNA, 21 bp, Artificial Sequence
  Primer
gacgctgaggcatgagagcat

SEQ ID NO: 53 - DNA, 21 bp, Artificial Sequence
  Primer
gacggcacggattgttattca

SEQ ID NO: 54 - DNA, 19 bp, Artificial Sequence
  Primer
gtgccagcmgccgcggtaa

SEQ ID NO: 55 - DNA, 20 bp, Artificial Sequence
  Primer
ggactachvgggtwtctaat

SEQ ID NO: 56 - DNA, 17 bp, Artificial Sequence
  Primer
ctcctggaaacgggtgg

SEQ ID NO: 57 - DNA, 22 bp, Artificial Sequence
  Primer
ggtgttcttcccgatatctaca

(19) United States
(12) Patent Application Publication
(10) Pub. No.: US 2016/0120915 A1
(43) Pub. Date: May 5, 2016

(54) METHODS FOR MANIPULATING IMMUNE RESPONSES BY ALTERING MICROBIOTA

(71) Applicant: NEW YORK UNIVERSITY, New York, NY (US)

(72) Inventors: Martin J. BLASER, New York, NY (US); Shingo YAMANISHI, Bunkyo-Ku (JP); Laura M. COX, Brooklyn, NY (US); Victoria E. RUIZ, Brooklyn, NY (US); Alexandra E. LIVANOS, New York, NY (US)

(73) Assignee: New York University, New York, NY (US)

(21) Appl. No.: 14/897,151

(22) PCT Filed: Jun. 10, 2014
(86) PCT No.: PCT/US14/41770

§ 371 Date: Dec. 9, 2015

Related U.S. Application Data
(60) Provisional application No. 61/833,356, filed on Jun. 10, 2013.
(60) Provisional application No. 61/926,046, filed on Jan. 10, 2014.

(51) Int. Cl. A61K 35/741; A23C 9/127; C12Q 1/68; A23C 9/123; A61K 31/43; A61K 31/7048

(52) U.S. Cl. CPC A61K 35/741; A61K 31/43; A61K 31/7048; C12Q 1/689; A23L 1/296; A23C 9/123; A23C 9/127; A23L 1/3014; A23Y 2300/00; A23V 2002/00

(57) ABSTRACT

The present invention relates to characterizing changes in mammalian microbiota associated with antibiotic treatments and various immunological conditions and related therapeutic methods. Therapeutic methods of the invention involve the use of probiotics, prebiotics, synbiotics, and antibiotics.

### CROSS-REFERENCE TO RELATED APPLICATIONS

This application claims priority to U.S. Provisional Application Ser. No. 61/833,356, filed Jun. 10, 2013, and U.S. Provisional Application Ser. No. 61/926,046, filed Jan. 10, 2014, which are herein incorporated by reference in their entirety.

### STATEMENT REGARDING FEDERALLY SPONSORED RESEARCH

Research and development leading to certain aspects of the present invention were supported, in part, by grants 1UL1RR029893, R01DK090989, and R01GM63270 from the National Center for Research Resources, National Institutes of Health. Accordingly, the U.S. government may have certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 10, 2014, is named seq243735-123.txt, and is 10,641 bytes in size.

### FIELD OF THE INVENTION

The present invention relates to characterizing changes in mammalian microbiota associated with antibiotic treatment and various immunological conditions and related therapeutic methods. Therapeutic methods of the invention involve the use of probiotics, prebiotics, and antibiotics.

### BACKGROUND OF THE INVENTION

Allergic and autoimmune diseases have been dramatically increasing in developed countries since the 1950s (Bach, NEJM, 2002, 347:911). Allergic diseases including asthma, allergic rhinitis, and atopic dermatitis and autoimmune disease including multiple sclerosis (MS), type 1 diabetes (T1D), and inflammatory bowel disease (IBD, Crohn's disease and ulcerative colitis), all are increasing in prevalence. These increases are too rapid to be explained by changes in known genetic polymorphisms or increasing diagnosis alone, suggesting the influence of environmental factors on disease development (Bach, NEJM, 2002, 347:911). The prevalence of celiac disease as well seems to be increasing in the United States (Ludvigsson et al., Am J Gastroenterol., 2013, 108:818).

Type 1 diabetes (T1D) incidence has been increasing in developed countries by 3-5% per year, with the greatest rate of increase in young children (Patterson et al., Lancet, 2009, 373:2027; Green et al., Diabetologia, 2001, 44:supp13:B3; Vehik et al., Diabetes Care. 2007, 30:503). The decreasing age of disease onset is associated with increased morbidity from secondary complications and further suggests increasing environmental pressures for T1D development (Dabelea et al., Lancet, 2009 373:1999). Rates of IBD are increasing worldwide, and incidence is particularly rising in developing countries with traditionally low-incidence. This may be due to consequences of changing life-styles to becoming more similar to developed countries (Burisch and Munkhold, Current Opinions in Gastroenterology, 2013, 29: 357). These changing life-styles often are associated with increases in antibiotics and elective caesarean sections.

Despite substantial research and numerous clinical trials, autoimmune diseases remain difficult to treat. Many current medications control symptoms but fail to modify disease progression. Furthermore, current treatments rarely provide a cure and must be taken long term, increasing toxicity. Furthermore, there are no effective methods to prevent the development of autoimmune and/or allergic diseases.

For type 1 diabetes (T1D), no treatments effectively prevent, alter disease progression, or cure the disease without intolerable toxicity. Currently, treatments involve insulin replacement for disease management, but these efforts fail to alter disease progression. A trial in the 1980s using cyclosporine showed that immune intervention could effectively delay T1D progression. However, for the treatment to be effective it must be taken from early in disease onset and continuously, and once the drug was removed, disease ensued. Due to adverse events and toxicity, this drug is not an acceptable form of treatment (Staeva et al., Diabetes, 2013, 62:9). Furthermore, there have been recent clinical trials attempting to alter disease progression using immune-modulatory biologics (including two with anti-CD3 and one with GAD-alum) that have failed to provide sufficient benefit or have acceptable toxicity (Staeva et al., Diabetes, 2013, 62:9). Therefore, new approaches are necessary.

Multiple sclerosis (MS) is an autoimmune disease targeting the myelin in the central nervous system resulting in significant disability. In young adults between ages 20-40 years old, MS is the leading cause of non-traumatic disability (Tullman et al., 2013, Am J Manag Care, 19:S21). Despite a large research effort, monitoring and treatment of MS remains difficult and insufficient for many patients (Filippi, Nature Reviews Neurology, 2011, 7:74).

Similar problems affect allergic diseases, including asthma. Clinical trials in humans have failed to show efficacy for numerous immunomodulatory drugs (Nguyen and Casale, Immunological Reviews, 2001, 242:258).

Although biologics including infliximab have improved the treatment of IBD, surgical rates for Crohn's disease remain very high, with about 50% of patients requiring surgery within 10 years after diagnosis. Additionally, despite treatments, these patients have decreased life expectancy (Burisch and Munkhold, Current Opinions in Gastroenterology, 2013, 29: 357).

Rheumatoid arthritis (RA) is a systemic autoimmune inflammatory disease that ultimately leads to joint destruction and decreased life expectancy (Nurmohamed et al., 2008, Arthritis Research and Therapy, 10:118). Traditional treatments with anti-inflammatory agents such NSAIDs and/or corticosteroids can reduce joint swelling and pain but do not alter disease progression. Methotrexate became the cornerstone of treatment in the mid 1980s although this treatment represented a great step forward, there remain patients who progressed and others unable to tolerate the long-term toxicities. New disease-modifying drugs and biologics such as TNFα blockers and IL-1 receptor antagonist are effective, however, these drugs are expensive and have not proven to be much superior than methotrexate. Furthermore, long-term cytokine inhibition can increase the risk of malignancy and infection (Nurmohamed et al., 2008, Arthritis Research and Therapy, 10:118; Hobbs et al., 2012, Rheumatology, 51 Suppl 6, vi21)

Numerous investigators have implicated T-cells and their secreted cytokines as both protective and pathologic agents in allergic, inflammatory, and autoimmune diseases. In allergic rhinitis, IL-4- and IL-5-producing CD4+ T-helper 2 (Th2) lymphocytes are elevated and induce IgE synthesis, eosinophil activation and mast cell degranulation (Broide, 2008, Ann. Rev. Med., 60:279-91). T-helper 1 (Th1) lymphocytes characterized by the production of IFNγ antagonize Th2 function and are considered protective. Use of IL-4 and IL-5 antagonists down-regulates Th2 immune responses (Nguyen et al., 2011, Immu. Rev., 242:258-271). Regulatory T-cells (Tregs) characterized by the expression of the transcription factor Foxp3 also down-regulate Th2 responses. Sublingual (SLIT) and subcutaneous (SCIT) immunotherapies, which provide gradual administration of allergens to induce a protective immune phenotype, induces a T-helper 1 (Th1) immune response and increases regulatory T-cells populations (Treg). In allergic rhinitis patients, serum IL-17A is elevated compared to healthy subjects suggesting that IL-17A expressing T-helper 17 (Th17) lymphocytes may have a pathologic role in allergic rhinitis. Th17 cells may have both protective and pathologic roles in allergic rhinitis, protective due to their ability to reduce matrix deposition by inducing production of metalloproteinases or pathologic by mediating IgE production and tissue inflammation and neutrophil recruitment (Broide, 2008, Ann. Rev. Med., 60:279-91).

Asthma and atopic dermatitis are also characterized by increased IgE levels with Th2 lymphocytes as the central disease mediators; however, IL-17- and IL-22-expressing T-helper lymphocytes play a substantial role in immunopathology, promoting an initial acute phase to a severe chronic phase (Lloyd et al., 2010, Nature Rev. Immun., 10:838-48). Th17 populations are up-regulated in asthmatic patients, which is correlated with disease severity.

Animal models have shown that airway sensitization primes both Th2 and Th17 populations, leading to increased neutrophil accumulation and airway hyper-responsiveness and inhibiting IL-17 responses during allergic sensitization (Lloyd et al., 2010, Nature Rev. Immun., 10:838-48). Treg populations suppress allergic inflammation and prevent airway remodeling (Lloyd et al., 2009, Immunity, 31:438-49). In cases of atopic dermatitis (AD), increased Th17 populations in peripheral blood of AD patients was associated with severity of the disease. IL-17 expression induced secretion of chemotactic and pro-inflammatory cytokines from keratinocytes, which may further promote disease progression (Koga et al., 2008, Jour. Inves. Derm., 128:2625-30).

T-helper lymphocytes have been associated with the progression of pancreatic and colorectal cancers. Th17 lymphocyte populations are significantly higher in tumor tissues than non-tumor tissues. An increase in Th17 is associated with a worse prognosis in colorectal cancer patients (Grivennikov et al., 2012, Nature, 491:254-8). In pancreatic cancer, an increase in Th2-tumor infiltrating lymphocytes expressing Gata3 is associated with poor prognosis versus patients with high T-bet expressing Th1 tumor-infiltrating lymphocyte populations (De Monte et al., 2011, J. Exp. Med., 208(3):469-78). The Th1-related cytokines: IFNγ and TNFα induce senescence through activation of p16INK4a in pancreatic beta cell lines (Braumüller et al., 2013, Nature, 494(7437):361-5).

Dysregulation of host immune responses is a causal factor for autoimmune diseases such as type 1 diabetes, Crohn's disease and rheumatoid arthritis. Type 1 diabetes (T1D) is a T-cell mediated disorder, both human and animal models have shown that T-cells are central to T1D pathogenesis. The NOD model is a widely used mouse model of spontaneous type 1 diabetes mellitus (Leiter, Curr Protoc Immunol 2001; Chapter 15:Unit 15 9; Anderson M S, Annu Rev Immunol 2005; 23:447-85). Adoptive transfer experiments using this model have shown that T-cells can transfer the diabetic phenotype from a diseased (or prediabetic) host to a NOD.SCID recipient. CD4+ and CD8+ populations mediate the diabetic phenotype. CD4+ Th1 and Th17 populations have been reported to be both protective and promote T1D (Mathis et al., 2011, Immunol. Rev., 245:239-49). Th17 populations are highly plastic and can convert to IFNγ producing Th1-like cells or can develop a regulatory phenotype. Foxp3+ Tregs were suggested to influence the progression of diabetes pathogenesis in NOD mice (Mathis et al., 2011, Immunol. Rev., 245:239-49).

Inflammatory bowel diseases such as Crohn's and ulcerative colitis are defined by abnormal immune responses against normal tissues. T-helper lymphocytes and their related cytokine have significant roles in the progression of these diseases. Pro-inflammatory Th17 populations are elevated in Crohn's and ulcerative colitis patients. However, animal studies also have shown that IL-17A has a protective effect in mouse models of colitis where IL-17A abrogation exacerbates DSS-induced coltis. Mice deficient in the anti-inflammatory cytokine IL-10 develop IBD-like enterocolitis, correlated with decreased regulatory T-cell function. (Kaser et al., 2009, Ann. Rev. Immun., 28:573-621). Thus, modulating a balance between pro-inflammatory and anti-inflammatory T cell subsets may play an important role in the treatment of these autoimmune diseases.

T-lymphocytes migrate to the thymus during fetal development, where they undergo both negative and positive selection. Naïve T cells egress to secondary lymphoid and mucosal tissues where they are primed by dendritic cells through antigen presentation and differentiate into effector T-helper subsets. The local cytokine environment influences T-helper cell differentiation and maturation. At birth, the T-cell repertoire is immunologically naïve and immunologic experience develops rapidly in the first three months of life, as the cells gain antigenic experience (Holt et al., 2000, Allergy, 55:688-697).

T-lymphocyte subsets have roles in both promoting and protecting against allergic, inflammatory, and autoimmune diseases. Although certain T-cell populations have been linked to disease phenotypes, there are few treatments available to safely manipulate T-cell populations. In asthma, IL-4, IL-5 antagonists and PPARγ agonists are used to decrease Th2 populations and TLR9 agonists are used to increase Th1 populations. However, these treatments do not always work and may make hosts more susceptible to disease. SLIT and SCIT immunotherapies to manipulate host immune response have long-lasting immunomodulating effects, however the proper doses and durations are yet to be optimized. (Broide, 2008, Ann. Rev. Med., 60:279-91). New methods are needed to safely manipulate host immune responses to prevent disease.

The human microbiota, our microbial residents, represent about 90% of the cells in the human body (Savage et al., 1977, Ann. Rev. Microbiol., 31:107-33). These bacterial communities have co-evolved with humans and have a complex, bidirectional interaction with the immune system (Hooper et al., 2012, Science, 336:1268-73). The interaction involves multiple components including the microbes, their metabolites, the epithelial cells, and cells of the adaptive and innate immune systems. Altering specific immunological components can cause significant effects on the intestinal microbiota. Mice deficient in toll-like receptor signaling molecule MyD88 produce high-titer of antibodies against commensal microbiota, inducing a hyper-activated adaptive immune response (Slack et al., 2009, Science, 325:617-20) and mice deficient in TLR-5 change the composition of the gut microbiota and exhibit features of metabolic syndrome (Vijay-Kumar et al., 2010, Science, 328:228-31). B-cells are significant contributors in the development of allergic and autoimmune diseases. In allergic disease states, immunoglobulin E (IgE) production by B-cells induces secretion of histamine and other pro-inflammatory molecules and promotes severity of disease. In autoimmune diseases such as type 1 diabetes, multiple sclerosis, rheumatoid arthritis and celiac disease, mature B-cells produce autoantibodies that induce tissue inflammation and damage. T-helper lymphocytes are known to either promote or prevent B-cell induced pathology in both allergic and autoimmune diseases. T-helper 2 (Th2) and T-helper 17 (Th17) lymphocytes are known to promote B-cell maturation and inflammation while T-helper 1 (Th1) lymphocytes inhibit Th2 mediated B-cell activation and maturation.

Recent work by Ivanov et al. (Cell, 2009, 139:485) has shown that segmented filamentous bacteria (SFB) adhere to the terminal ileum of mice and play a role in the induction of Th17 cells. However, SFB has not been definitively identified in the human gastrointestinal track (Sczesnak et al., 2011, 10: 260; Jonsson et al. 2013, FEMS, 342-24).

It is known that macrolides have immunomodulatory effects without affecting homeostatic immunity (Kanoh et al., 2010, Clin. Microbio. Rev., 23:590-615). Macrolides have been shown to treat steroid-resistant forms of asthma and clarithromycin treatment reduced airway hyperresponsives (AHR) and neutrophilic inflammation (Simpson et al., 2008, Am. J. Respir. Crit. Care Med., 177:148-155). Tylosin, a macrolide, has anti-inflammatory properties in ITS-induced macrophages in vitro (Cao et al., 2006, Inter. Jour. Antimicro. Agen., 27: 431-438).

### SUMMARY OF THE INVENTION

As specified in the Background section above, there is a great need in the art (i) to understand the impact that mammalian bacterial microbiota has on the host immunity and health and (ii) to employ such knowledge in development of new therapeutics. There is further a great need in the art to treat such diseases as type 1 diabetes (T1D), multiple sclerosis (MS), rheumatoid arthritis (RA), asthma, inflammatory bowel diseases (e.g., ulcerative colitis, Crohn's disease, celiac disease), atopic diseases (e.g., atopic dermatitis (eczema), allergic rhinitis and conjunctivitis, and other allergic diseases), Systemic Lupus Erythemtosis (SLE), amyotrophic lateral sclerosis (ALS), sarcoidosis, scleroderma, thyroiditis, intestinal pulmonary fibrosis, psoriasis, graft versus host disease, autism, oral candidiasis, vaginal candidiasis, and related disorders.

The present invention addresses these and other needs by characterizing specific changes in mammalian bacterial microbiota associated with antibiotic treatment and specific diseases and related diagnostic and therapeutic methods.

In one embodiment, the invention provides a method for decreasing Th17 and/or Treg and/or Th2 cell differentiation in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial species is from the order Bacteroidales. In one specific embodiment, said bacterial species is from a family selected from the group consisting of Lachnospiraceae, Clostridiaceae, Bacteroidaceae, Bifidobacteriaceae and Enterobacteriaceae. In one specific embodiment, said bacterial species is from a genus selected from the group consisting of Akkermansia, Odoribacter, Enterococcus, Candidatus Arthromitus (segmented filamentous bacteria), Bacteroides, Blautia, Bifidobacterium, and Proteus. In one specific embodiment, said bacterial species is selected from the group consisting of Bacteroides uniformis, Blautia producta, Bifidobacterium pseudolongum, and Akkermansia muciniphila.

In a related embodiment, the invention provides a method for decreasing Th17 and/or Treg and/or Th2 cell differentiation in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition results in a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial strain is from the order Bacteroidales. In one specific embodiment, said bacterial strain is from a family selected from the group consisting of Lachnospiraceae, Clostridiaceae, Bacteroidaceae, Bifidobacteriaceae and Enterobacteriaceae. In one specific embodiment, said bacterial strain is from a genus selected from the group consisting of Akkermansia, Odoribacter, Enterococcus, Candidatus Arthromitus (segmented filamentous bacteria), Bacteroides, Blautia, Bifidobacterium, and Proteus. In one specific embodiment, said bacterial strain is from the species selected from the group consisting of Bacteroides uniformis, Blautia producta, Bifidobacterium pseudolongum, and Akkermansia muciniphila.

In another embodiment, the invention provides a method for treating a disease treatable by decreasing Th17 and/or Treg and/or Th2 cell differentiation in the intestine and/or tonsils and/or waldeyer's ring and/or vagina of a mammal in need thereof (e.g., multiple sclerosis (MS), rheumatoid arthritis (RA), asthma, inflammatory bowel diseases [e.g., ulcerative colitis, Crohn's disease, celiac disease], atopic diseases [e.g., atopic dermatitis (eczema), allergic rhinitis, conjunctivitis, and other allergic diseases], Systemic Lupus Erythematosis (SLE), amyotrophic lateral sclerosis (ALS), sarcoidosis, scleroderma, thyroiditis, idiopathic pulmonary fibrosis, psoriasis, and graft versus host disease), said method comprising enhancing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial species is from the order Bacteroidales. In one specific embodiment, said bacterial species is from a family selected from the group consisting of Lachnospiraceae, Clostridiaceae, Bacteroidaceae, Bifidobacteriaceae and Enterobacteriaceae. In one specific embodiment, said bacterial species is from a genus selected from the group consisting of Akkermansia, Odoribacter, Enterococcus, Candidatus Arthromitus (segmented filamentous bacteria), Bacteroides, Blautia, Bifidobacterium, and Proteus. In one specific embodiment, said bacterial species is selected from the group consisting of Bacteroides uniformis, Blautia producta, Bifidobacterium pseudolongum, and Akkermansia muciniphila.

In a related embodiment, the invention provides a method for treating a disease treatable by decreasing Th17 and/or Treg and/or Th2 cell differentiation in the intestine and/or tonsils and/or waldeyer's ring and/or vagina of a mammal in need thereof (e.g., multiple sclerosis (MS), rheumatoid arthritis (RA), asthma, inflammatory bowel diseases [e.g., ulcerative colitis, Crohn's disease, celiac disease], atopic diseases [e.g., atopic dermatitis (eczema), allergic rhinitis, conjunctivitis, and other allergic diseases], Systemic Lupus Erythematosis (SLE), amyotrophic lateral sclerosis (ALS), sarcoidosis, scleroderma, thyroiditis, idiopathic pulmonary fibrosis, psoriasis, and graft versus host disease), said method comprising administering to said mammal a therapeutically effective amount of a probiotic composition comprising at least one bacterial strain, or a combination of several strains, wherein the administration of the probiotic composition results in a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial strain is from the order Bacteroidales. In one specific embodiment, said bacterial strain is from a family selected from the group consisting of Lachnospiraceae, Clostridiaceae, Bacteroidaceae, Bifidobacteriaceae and Enterobacteriaceae. In one specific embodiment, said bacterial strain is from a genus selected from the group consisting of Akkermansia, Odoribacter, Enterococcus, Candidatus Arthromitus (segmented filamentous bacteria), Bacteroides, Blautia, Bifidobacterium, and Proteus. In one specific embodiment, said bacterial strain is from the species selected from the group consisting of Bacteroides uniformis, Blautia producta, Bifidobacterium pseudolongum, and Akkermansia muciniphila.

In another embodiment, the invention provides a method for decreasing the expression of at least one serum amyloid A (SAA) gene in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes a decrease in the expression of said SAA gene(s) in the epithelium and/or lamina propria of the intestines and/or tonsils and/or waldeyer's ring and/or vagina of a mammal. In one specific embodiment, said bacterial species is from a genus selected from the group consisting of Akkermansia, Sutterella, and Blautia. In one specific embodiment, said bacterial species is Akkermansia muciniphila or Blautia producta.

In a related embodiment, the invention provides a method for decreasing the expression of at least one serum amyloid A (SAA) gene in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition causes a decrease in the expression of said SAA gene(s) in the epithelium and/or lamina propria of the intestines and/or tonsils and/or waldeyer's ring and/or vagina of a mammal. In one specific embodiment, said bacterial strain is from a genus selected from the group consisting of Akkermansia, Sutterella, and Blautia. In one specific embodiment, said bacterial strain is from the species Akkermansia muciniphila or species Blautia producta.

In a further embodiment, the invention provides a method for treating a disease treatable by decreasing the expression of at least one serum amyloid A (SAA) gene in the intestine and/or tonsils and/or waldeyer's ring and/or vagina of a mammal in need thereof (e.g., multiple sclerosis (MS), rheumatoid arthritis (RA), asthma, inflammatory bowel diseases [e.g., ulcerative colitis, Crohn's disease, celiac disease], atopic diseases [e.g., atopic dermatitis (eczema), allergic rhinitis, conjunctivitis, and other allergic diseases], Systemic Lupus Erythematosis (SLE), amyotrophic lateral sclerosis (ALS), sarcoidosis, scleroderma, thyroiditis, idiopathic pulmonary fibrosis, psoriasis, and graft versus host disease), said method comprising enhancing growth or activity of at least one bacterial species in the intestinal and/or oropharyngeal and/or vaginal microbiota of said mammal, wherein said bacterial species causes a decrease in the expression of said SAA gene(s) in the epithelium and/or lamina propria of the intestines and/or tonsils and/or waldeyer's ring and/or vagina of a mammal. In one specific embodiment, said bacterial species is from a genus selected from the group consisting of Akkermansia, Sutterella, and Blautia. In one specific embodiment, said bacterial species is Akkermansia muciniphila or Blautia producta.

In a related embodiment, the invention provides a method for treating a disease treatable by decreasing the expression of at least one serum amyloid A (SAA) gene in the intestine and/or tonsils and/or waldeyer's ring and/or vagina of a mammal in need thereof (e.g., multiple sclerosis (MS), rheumatoid arthritis (RA), asthma, inflammatory bowel diseases [e.g., ulcerative colitis, Crohn's disease, celiac disease], atopic diseases [e.g., atopic dermatitis (eczema), allergic rhinitis, conjunctivitis, and other allergic diseases], Systemic Lupus Erythematosis (SLE), amyotrophic lateral sclerosis (ALS), sarcoidosis, scleroderma, thyroiditis, idiopathic pulmonary fibrosis, psoriasis, and graft versus host disease), said method comprising administering to said mammal a therapeutically effective amount of a probiotic composition comprising at least one bacterial strain, or a combination of several strains, wherein the administration of the probiotic composition causes a decrease in the expression of said SAA gene(s) in the epithelium and/or lamina propria of the intestines and/or tonsils and/or waldeyer's ring and/or vagina of a mammal. In one specific embodiment, said bacterial strain is from a genus selected from the group consisting of Akkermansia, Sutterella, and Blautia. In one specific embodiment, said bacterial strain is from the species Akkermansia muciniphila or species Blautia producta.

In a separate embodiment, the invention provides a method for increasing Th17 and/or Treg and/or Th2 cell differentiation in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial species is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae. In one specific embodiment, said bacterial species is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella. In one specific embodiment, said bacterial species is selected from the group consisting of Bacteroides uniformis, Blautia producta, Bifidobacterium pseudolongum, and Lactobacillus reuteri.

In a related embodiment, the invention provides a method for increasing Th17 and/or Treg and/or Th2 cell differentiation in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition results in an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial strain is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae. In one specific embodiment, said bacterial strain is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella. In one specific embodiment, said bacterial strain is from the species selected from the group consisting of Bacteroides uniformis, Blautia producta, Bifidobacterium pseudolongum, and Lactobacillus reuteri.

In another embodiment, the invention provides a method for treating a disease treatable by increasing Th17 and/or Treg and/or Th2 cell differentiation in the intestine and/or tonsils and/or waldeyer's ring and/or vagina of a mammal in need thereof (e.g., type 1 diabetes (T1D), Crohn's disease, celiac disease, autism, oral candidiasis, vaginal candidiasis), said method comprising enhancing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial species is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae. In one specific embodiment, said bacterial species is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella. In one specific embodiment, said bacterial species is selected from the group consisting of Bacteroides uniformis, Blautia producta, Bifidobacterium pseudolongum, and Lactobacillus reuteri.

In a related embodiment, the invention provides a method for treating a disease treatable by increasing Th17 and/or Treg and/or Th2 cell differentiation in the intestine and/or tonsils and/or waldeyer's ring and/or vagina of a mammal in need thereof (e.g., type 1 diabetes (T1D), Crohn's disease, celiac disease, autism, oral candidiasis, vaginal candidiasis), said method comprising administering to said mammal a therapeutically effective amount of a probiotic composition comprising at least one bacterial strain, or a combination of several strains, wherein the administration of the probiotic composition results in an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector. In one specific embodiment, said bacterial strain is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae. In one specific embodiment, said bacterial strain is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella. In one specific embodiment, said bacterial strain is from the species selected from the group consisting of Bacteroides uniformis, Blautia producta, Bifidobacterium pseudolongum, and Lactobacillus reuteri.

In another embodiment, the invention provides a method for increasing the expression of at least one serum amyloid A (SAA) gene in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes an increase in the expression of said SAA gene(s) in the epithelium and/or lamina propria of the intestines and/or tonsils and/or waldeyer's ring and/or vagina of a mammal. In one specific embodiment, said bacterial species is from the order RF39 (class Mollicutes). In one specific embodiment, said bacterial species is from a genus selected from the group consisting of Oscillospira, Odoribacter, and Bifidobacterium.

In a related embodiment, the invention provides a method for increasing the expression of at least one serum amyloid A (SAA) gene in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition causes an increase in the expression of said SAA gene(s) in the epithelium and/or lamina propria of the intestines and/or tonsils and/or waldeyer's ring and/or vagina of a mammal. In one specific embodiment, said bacterial strain is from the order RF39 (class Mollicutes). In one specific embodiment, said bacterial strain is from a genus selected from the group consisting of Oscillospira, Odoribacter, and Bifidobacterium.

In a further embodiment, the invention provides a method for treating a disease treatable by increasing the expression of at least one serum amyloid A (SAA) gene in the intestine and/or tonsils and/or waldeyer's ring and/or vagina of a mammal in need thereof (e.g., type 1 diabetes (T1D), Crohn's disease, celiac disease, autism, oral candidiasis, vaginal candidiasis), said method comprising enhancing growth or activity of at least one bacterial species in the intestinal and/or oropharyngeal and/or vaginal microbiota of said mammal, wherein said bacterial species causes an increase in the expression of said SAA gene(s) in the epithelium and/or lamina propria of the intestines and/or tonsils and/or waldeyer's ring and/or vagina of a mammal. In one specific embodiment, said bacterial species is from the order RF39 (class Mollicutes). In one specific embodiment, said bacterial species is from a genus selected from the group consisting of Oscillospira, Odoribacter, and Bifidobacterium.

In a related embodiment, the invention provides a method for treating a disease treatable by increasing the expression of at least one serum amyloid A (SAA) gene in the intestine and/or tonsils and/or waldeyer's ring and/or vagina of a mammal in need thereof (e.g., type 1 diabetes (T1D), Crohn's disease, celiac disease, autism, oral candidiasis, vaginal candidiasis), said method comprising administering to said mammal a therapeutically effective amount of a probiotic composition comprising at least one bacterial strain, or a combination of several strains, wherein the administration of the probiotic composition causes an increase in the expression of said SAA gene(s) in the epithelium and/or lamina propria of the intestines and/or tonsils and/or waldeyer's ring and/or vagina of a mammal. In one specific embodiment, said bacterial strain is from the order RF39 (class Mollicutes). In one specific embodiment, said bacterial strain is from a genus selected from the group consisting of Oscillospira, Odoribacter, and Bifidobacterium.

In any of the above methods, enhancing growth or activity of at least one bacterial species in the microbiota can comprise, for example, administering to said mammal a therapeutically effective amount of one or more prebiotic agents which enhance growth or activity of said bacterial species. Non-limiting examples of useful prebiotic agents include fructooligosaccharides (e.g., oligofructose, inulin, inulin-type fructans), galactooligosaccharides, N-acetylglucosamine, N-acetylgalactosamine, glucose, penta-saccharides, hexa-saccharides (e.g., arabinose, maltose, lactose, sucrose, cellobiose), amino acids, alcohols, and resistant starch (RS).

In any of the above methods, enhancing growth or activity of at least one bacterial species in the microbiota can comprise, for example, administering to said mammal a therapeutically effective amount of a probiotic composition comprising at least one bacterial strain of said bacterial species.

Thus, in conjunction with the above methods, the invention also provides probiotic compositions comprising one or more bacterial strains specified above.

Non-limiting examples of useful probiotic compositions include live bacterial cells (including bacterial analogues and conditionally lethal bacterial strains), killed bacterial cells, and bacterial extract.

Probiotic compositions useful in the methods of the invention can further comprise (i) a buffering agent (e.g., saline, sodium bicarbonate, milk, yogurt, infant formula, and other dairy products) and/or (ii) one or more prebiotic agents which enhance growth or activity of the bacterial strain.

Prebiotic and/or probiotic compositions of the invention can be administered via various routes of administration, including, for example, orally, rectally, fecally (by enema), or via naso/oro-gastric gavage.

In a separate embodiment, the invention provides a method for decreasing Th17 and/or Treg and/or Th2 cell differentiation in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal. In one specific embodiment, said bacterial species is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae. In one specific embodiment, said bacterial species is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella. In one specific embodiment, said bacterial species is selected from the group consisting of Bacteroides uniformis, Blautia producta, Bifidobacterium pseudolongum, and Lactobacillus reuteri. In one specific embodiment, the increase in Th17 and/or Treg and/or Th2 cell differentiation is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

In another embodiment, the invention provides a method for treating a disease treatable by decreasing Th17 and/or Treg and/or Th2 cell differentiation in the intestine and/or tonsils and/or waldeyer's ring and/or vagina of a mammal in need thereof (e.g., multiple sclerosis (MS), rheumatoid arthritis (RA), asthma, inflammatory bowel diseases [e.g., ulcerative colitis, Crohn's disease, celiac disease], atopic diseases [e.g., atopic dermatitis (eczema), allergic rhinitis, conjunctivitis, and other allergic diseases], Systemic Lupus Erythematosis (SLE), amyotrophic lateral sclerosis (ALS), sarcoidosis, scleroderma, thyroiditis, idiopathic pulmonary fibrosis, psoriasis, and graft versus host disease), said method comprising suppressing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal. In one specific embodiment, said bacterial species is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae. In one specific embodiment, said bacterial species is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella. In one specific embodiment, said bacterial species is selected from the group consisting of Bacteroides uniformis, Blautia producta, Bifidobacterium pseudolongum, and Lactobacillus reuteri. In one specific embodiment, the increase in Th17 and/or Treg and/or Th2 cell differentiation is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

In yet another embodiment, the invention provides a method for decreasing the expression of at least one serum amyloid A (SAA) gene in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes an increase in the expression of said SAA gene(s) in the intestine and/or tonsils and/or waldeyer's ring and/or vagina of the mammal. In one specific embodiment, said bacterial species is from the order RF39 (class Mollicutes). In one specific embodiment, said bacterial species is from a genus selected from the group consisting of Oscillospira, Odoribacter, and Bifidobacterium.

In a further embodiment, the invention provides a method for treating a disease treatable by decreasing the expression of at least one serum amyloid A (SAA) gene in the intestine and/or tonsils and/or waldeyer's ring and/or vagina of a mammal in need thereof (e.g., multiple sclerosis (MS), rheumatoid arthritis (RA), asthma, inflammatory bowel diseases [e.g., ulcerative colitis, Crohn's disease, celiac disease], atopic diseases [e.g., atopic dermatitis (eczema), allergic rhinitis, conjunctivitis, and other allergic diseases], Systemic Lupus Erythematosis (SLE), amyotrophic lateral sclerosis (ALS), sarcoidosis, scleroderma, thyroiditis, idiopathic pulmonary fibrosis, psoriasis, and graft versus host disease), said method comprising suppressing growth or activity of at least one bacterial species in the intestinal and/or oropharyngeal and/or vaginal microbiota of said mammal, wherein said bacterial species causes an increase in the expression of said SAA gene(s) in the intestine and/or tonsils and/or waldeyer's ring and/or vagina of the mammal. In one specific embodiment, said bacterial species is from the order RF39 (class Mollicutes). In one specific embodiment, said bacterial species is from a genus selected from the group consisting of Oscillospira, Odoribacter, and Bifidobacterium.

In another embodiment, the invention provides a method for increasing Th17 and/or Treg and/or Th2 cell differentiation in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal. In one specific embodiment, said bacterial species is from the order Bacteroidales. In one specific embodiment, said bacterial species is from a family selected from the group consisting of Lachnospiraceae, Clostridiaceae, Bacteroidaceae, Bifidobacteriaceae and Enterobacteriaceae. In one specific embodiment, said bacterial species is from a genus selected from the group consisting of Akkermansia, Odoribacter, Enterococcus, Candidatus Arthromitus (segmented filamentous bacteria), Bacteroides, Blautia, Bifidobacterium, and Proteus. In one specific embodiment, said bacterial species is Akkermansia muciniphila. In one specific embodiment, the decrease in Th17 and/or Treg and/or Th2 cell differentiation is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

In a further embodiment, the invention provides a method for treating a disease treatable by increasing Th17 and/or Treg and/or Th2 cell differentiation in the intestine and/or tonsils and/or waldeyer's ring and/or vagina of a mammal in need thereof (e.g., type 1 diabetes (T1D), Crohn's disease, celiac disease, autism, oral candidiasis, vaginal candidiasis), said method comprising suppressing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal. In one specific embodiment, said bacterial species is from the order Bacteroidales. In one specific embodiment, said bacterial species is from a family selected from the group consisting of Lachnospiraceae, Clostridiaceae, Bacteroidaceae, Bifidobacteriaceae and Enterobacteriaceae. In one specific embodiment, said bacterial species is from a genus selected from the group consisting of Akkermansia, Odoribacter, Enterococcus, Candidatus Arthromitus (segmented filamentous bacteria), Bacteroides, Blautia, Bifidobacterium, and Proteus. In one specific embodiment, said bacterial species is Akkermansia muciniphila. In one specific embodiment, the decrease in Th17 and/or Treg and/or Th2 cell differentiation is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

In another embodiment, the invention provides a method for increasing the expression of at least one serum amyloid A (SAA) gene in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes a decrease in the expression of said SAA gene(s) in the intestine and/or tonsils and/or waldeyer's ring and/or vagina of the mammal. In one specific embodiment, said bacterial species is from a genus selected from the group consisting of Akkermansia, Sutterella, and Blautia. In one specific embodiment, said bacterial species is Akkermansia muciniphila or Blautia producta.

In yet another embodiment, the invention provides a method for treating a disease treatable by increasing the expression of at least one serum amyloid A (SAA) gene in the intestine and/or tonsils and/or waldeyer's ring and/or vagina of a mammal in need thereof (e.g., type 1 diabetes (T1D), Crohn's disease, celiac disease, autism, oral candidiasis, vaginal candidiasis), said method comprising suppressing growth or activity of at least one bacterial species in the intestinal and/or oropharyngeal and/or vaginal microbiota of said mammal, wherein said bacterial species causes a decrease in the expression of said SAA gene(s) in the intestine and/or tonsils and/or waldeyer's ring and/or vagina of the mammal. In one specific embodiment, said bacterial species is from a genus selected from the group consisting of Akkermansia, Sutterella, and Blautia. In one specific embodiment, said bacterial species is Akkermansia muciniphila or Blautia producta.

Suppressing growth or activity of at least one bacterial species in the microbiota according to any of the above methods involving such suppression can be achieved, e.g., by administering an antibiotic. In one specific embodiment, the antibiotic is administered in a therapeutic dose. In another specific embodiment, the antibiotic is administered in a sub-therapeutic dose. Non-limiting examples of antibiotics useful in the methods of the invention include beta-lactams (e.g., Penicillin VK, Penicillin G, Amoxicillin trihydrate), nitroimidazoles, macrolides (e.g., Tylosin tartrate, Erythromycin, Azithromycin, and Clarithromycin), tetracyclines, glycopeptides (e.g., Vancomycin), and fluoroquinolones. In one specific embodiment, the method comprises administering Penicillin VK or Penicillin G at 1 mg/kg body weight per day for at least four weeks of life. In another specific embodiment, the method comprises administering Amoxicillin trihydrate at 25 mg/kg body weight per day for 1 to 3 treatments each lasting 3 to 5 days. In yet another specific embodiment, the method comprises administering Tylosin tartrate at 50 mg/kg body weight per day for 1 to 3 treatments each lasting 3 to 5 days.

In one specific embodiment, suppressing growth or activity of at least one bacterial species in the microbiota results in a decrease in expression of one or more genes selected from the group consisting of Rab30, Areg, Flrt3, Arl5b, Maff, Dusp14, Zfand2a, Chka, Phlda1, Ereg, Tnfrsf12a, Il1rn, Il1rL1, Acs13, Slc2a1, S100a14, Klf4, and Gjb3, in the intestinal and/or oropharyngeal and/or vaginal epithelium and lamina propria of said mammal.

In one specific embodiment, suppressing growth or activity of at least one bacterial species in the microbiota results in an increase in expression of one or more genes selected from the group consisting of Saa1, G6pc, Edn2, Bhmt, Gzma, Tnfsf10, Saa2, Tppp, Dio1, Tifa, Irf1, Cd38, Cc120, Socs2, Clec2d, Ccl128, and Il17rd, in the intestinal and/or oropharyngeal and/or vaginal epithelium and lamina propria of said mammal.

Non-limiting examples of cytokines, chemokines, and effectors monitored in the methods of the invention include:

(i) Th17-specific cytokines, chemokines, or effectors such as, e.g., IL-17A, IL-17F, IL-21, IL-22, IL-23, CCL20, beta defensin 4, CD-161, and CCR6; defensin β1, Reg3γ, and Relmβ;
(ii) Th2-specific cytokines such as, e.g., IL-4, IL-5, IL-9, and IL-13;
(iii) Treg-specific cytokines such as, e.g., TGFβ, IL-10, and IL-35.

In any of the methods of the invention involving the SAA gene, such gene can be SAA1, SAA2, or SAA3. For non-limiting examples of corresponding sequences see, e.g., GenBank Accession Nos. NM_009117.3 (mouse SAA1 mRNA), NM_000331.4 (human SAA1 mRNA), NM_011314.2 (mouse SAA2 mRNA), NM_030754.4 (human SAA2 mRNA), NM_011315.3 (mouse SAA3 mRNA), NR_026576.1 (human SAA3 pseudogene).

In a separate embodiment, the invention provides a method for determining a risk for developing type 1 diabetes (T1D) in a subject, said method comprising:

(a) determining a relative abundance of one or more Bifidobacterium species in a gastrointestinal microbiota sample obtained from the subject;
(b) comparing the one or more relative abundances determined in step (a) to a healthy control relative abundance for the same species, and
(c) (i) determining that the subject is at high risk for developing type 1 diabetes if the relative abundance of the one or more Bifidobacterium species in the gastrointestinal microbiota sample from the subject is decreased at least two-fold as compared to the control, or (ii) determining that the subject is not at high risk for developing type 1 diabetes if the relative abundance of the one or more Bifidobacterium species in the gastrointestinal microbiota sample from the subject is not decreased as compared to the control.

In another embodiment, the invention provides a method for determining a risk for developing type 1 diabetes in a subject, said method comprising:

(a) determining a relative abundance of Akkermansia mucinophilia in a gastrointestinal microbiota sample obtained from the subject;
(b) comparing the relative abundance determined in step (a) to a healthy control relative abundance for the same species, and
(c) (i) determining that the subject is at high risk for developing type 1 diabetes if the relative abundance of Akkermansia mucinophilia in the gastrointestinal microbiota sample from the subject is increased at least two-fold as compared to the control, or (ii) determining that the subject is not at high risk for developing type 1 diabetes if the relative abundance of Akkermansia mucinophilia in the gastrointestinal microbiota sample from the subject is not increased as compared to the control.

In one embodiment of the above two methods for determining a risk for developing T1D, the subject is a child. In one embodiment of these methods, the subject has not been exposed to antibiotics for at least one month before the microbiota sample is obtained.

Non-limiting examples of gastrointestinal microbiota samples useful in the above two methods for determining a risk for developing T1D, include fecal, cecal, and ileal samples. In one embodiment of these methods, the method further comprises obtaining the gastrointestinal microbiota sample from the subject prior to step (a). In one embodiment, the method further comprises subjecting the gastrointestinal microbiota sample to a treatment to maintain DNA integrity. Non-limiting examples of useful treatments to maintain DNA integrity include freezing of the sample or adding a preservative (e.g., RNAlater or Paxgene, or drying over silica gel or on Whatman FTA cards).

In one embodiment of the above two methods for determining a risk for developing T1D, determining the relative abundance of the bacterial species comprises a method selected from the group consisting of quantitative polymerase chain reaction (qPCR), sequencing of bacterial 16S rRNA, and shotgun metagenome sequencing.

In one embodiment of the above two methods for determining a risk for developing T1D, the healthy control relative abundance is a predetermined standard. In another embodiment of these methods, the healthy control relative abundance is obtained using a healthy subject or several healthy subjects of the same gender, age and ethnicity as the subject who is being diagnosed for T1D. In one specific embodiment, the healthy subject has not been exposed to antibiotics for at least one month before the microbiota sample is obtained.

In one embodiment of the above two methods for determining a risk for developing T1D, the method further comprises treating the subject who has been determined to be at high risk for developing T1D with a diabetes treatment. In one embodiment, the diabetes treatment comprises administering to the subject a therapeutically effective amount of a compound or composition, wherein said compound or composition increases the abundance of at least one Bifidobacterium species in the gastrointestinal microbiota of the subject. In one specific embodiment, said compound is a prebiotic. In one specific embodiment, said composition comprises a probiotic and/or a prebiotic. Non-limiting examples of useful prebiotics include, e.g., a fructooligosaccharide (FOS), inulin, a galactooligosaccharide (GOS), a human milk oligosaccharide (HMO), Lacto-N-neotetraose, D-Tagatose, xylo-oligosaccharide (XOS), an arabinoxylan-oligosaccharide (AXOS), and any mixtures thereof. Non-limiting examples of useful probiotics include, e.g., live cells, conditionally lethal cells, spores, inactivated cells, killed cells, and a cell extract. In one specific embodiment, said probiotic comprises at least one Bifidobacterium strain. In another embodiment, the diabetes treatment comprises administering to the subject a therapeutically effective amount of a compound or composition, wherein said compound or composition decreases the abundance of Akkermansia mucinophilia in the gastrointestinal microbiota of the subject. In one specific embodiment, said compound is a narrow spectrum antibiotic (or said composition comprises a narrow spectrum antibiotic).

In a further embodiment, the invention provides a method for preventing or delaying onset or decreasing severity of type 1 diabetes (T1D) in a subject in need thereof, said method comprising administering to the subject a therapeutically effective amount of a compound or composition, wherein said compound or composition increases the abundance of at least one Bifidobacterium species in the gastrointestinal microbiota of the subject (e.g., fecal, cecal, and/or ileal microbiota). In one specific embodiment, the subject is at high risk of developing T1D.

In a separate embodiment, the invention provides a method for enhancing a mucosal IgA response in a subject in need thereof, said method comprising administering to the subject a therapeutically effective amount of a compound or composition, wherein said compound or composition increases the abundance of at least one Bifidobacterium species in a mucosal microbiota of the subject. In one specific embodiment, the mucosal microbiota is gastrointestinal microbiota (e.g., fecal, cecal, and/or ileal microbiota).

In another embodiment, the invention provides a method for enhancing SAA1 gene expression and/or intestinal barrier function in a subject in need thereof, said method comprising administering to the subject a therapeutically effective amount of a compound or composition, wherein said compound or composition increases the abundance of at least one Bifidobacterium species in a mucosal microbiota of the subject. In one specific embodiment, the mucosal microbiota is gastrointestinal microbiota (e.g., fecal, cecal, and/or ileal microbiota).

In a further embodiment, the invention provides a method for enhancing interferon-gamma (IFNγ) production in the spleen in a subject in need thereof, said method comprising administering to the subject a therapeutically effective amount of a compound or composition, wherein said compound or composition increases the abundance of at least one Bifidobacterium species in a mucosal microbiota of the subject. In one specific embodiment, the mucosal microbiota is gastrointestinal microbiota (e.g., fecal, cecal, and/or ileal microbiota).

In yet another embodiment, the invention provides a method for preventing or delaying onset or decreasing severity of a disease selected from the group consisting of Celiac disease, Graves disease, and Hashimoto's thyroiditis in a subject in need thereof, said method comprising administering to the subject a therapeutically effective amount of a compound or composition, wherein said compound or composition increases the abundance of at least one Bifidobacterium species in the gastrointestinal microbiota of the subject (e.g., fecal, cecal, and/or ileal microbiota).

In a further embodiment, the invention provides a method for preventing or delaying onset or decreasing severity of an early life male-dominated disease in a subject in need thereof, said method comprising administering to the subject a therapeutically effective amount of a compound or composition, wherein said compound or composition increases the abundance of at least one Bifidobacterium species in the gastrointestinal microbiota of the subject (e.g., fecal, cecal, and/or ileal microbiota). In one specific embodiment, the early life male-dominated disease is autism or attention-deficit disorder (ADD).

In one embodiment of the above treatment methods (i.e., a method for preventing or delaying onset or decreasing severity of T1D, or Celiac disease/Graves disease/Hashimoto's thyroiditis, or early life male-dominated diseases; a method for enhancing a mucosal IgA response; a method for enhancing SAA1 gene expression and/or intestinal barrier function; a method for enhancing IFNγ production in the spleen), said compound is a prebiotic. In one embodiment of these methods, said composition comprises a probiotic and/or a prebiotic. Non-limiting examples of useful prebiotics include, e.g., a fructooligosaccharide (FOS), inulin, a galactooligosaccharide (GOS), a human milk oligosaccharide (HMO), Lacto-N-neotetraose, D-Tagatose, a xylo-oligosaccharide (XOS), an arabinoxylan-oligosaccharide (AXOS), and any mixtures thereof. Non-limiting examples of useful probiotics include, e.g., live cells, conditionally lethal cells, spores, inactivated cells, killed cells, and a cell extract. In one specific embodiment, said probiotic comprises at least one Bifidobacterium strain. In one specific embodiment, the probiotic composition further comprises a buffering agent (e.g., sodium bicarbonate, dairy drinks [e.g., milk, yougurt and kefir], and infant formula). In one specific embodiment, the probiotic composition is administered conjointly with a prebiotic which stimulates growth and/or metabolic activity of bacteria contained in the probiotic composition. The probiotic and prebiotic can be administered in one composition, or simultaneously as two separate compositions, or sequentially. In one specific embodiment, said compound is a narrow spectrum antibiotic, which inhibits growth of one or more suppressors or competitors of Bifidobacterium (or said composition comprises a narrow spectrum antibiotic, which inhibits growth of one or more suppressors or competitors of Bifidobacterium). The compound or composition can be administered, for example (but not limited to), orally, rectally, fecally, or via naso/oro-gastric gavage. In one specific embodiment, the compound or composition is contained within an infant formula. In one specific embodiment, the compound or composition is administered to a pregnant woman who is in active labor (e.g., to the vaginal area of the woman). In one specific embodiment, the compound or composition is administered to a newborn child (e.g., to the child's mouth and/or skin) In one specific embodiment, the compound or composition is administered to a newborn child who was born via a C-section (e.g., to the child's mouth and/or skin) In one specific embodiment, the compound or composition is applied to mother's nipples during breastfeeding.

In another embodiment, the invention provides a method for preventing or delaying onset or decreasing severity of type 1 diabetes (T1D) in a subject in need thereof, said method comprising administering to the subject a therapeutically effective amount of a compound or composition, wherein said compound or composition decreases the abundance of Akkermansia mucinophilia in the gastrointestinal microbiota of the subject. In one specific embodiment, said compound is a narrow spectrum antibiotic (or said composition comprises a narrow spectrum antibiotic).

In one specific embodiment of any of the methods of the invention, the mammal/subject is human. In one specific embodiment of any of the methods of the invention, the mammal/subject is a child.

### BRIEF DESCRIPTION OF THE DRAWINGS

The patent application file contains at least one drawing executed in color.

FIG. 1 is a schematic of TimeSTAT study design.

FIG. 2 is a schematic of EarlySTAT study design and study groups for gene expression analysis.

FIGS. 3A-B show TimeSTAT: the effect of early or late STAT exposure on immunologic markers. Expression of immunological markers in ileal tissue was measured in (A) female, and (B) male mice. (A) In female mice, there were significant reductions in Th17 transcription factor (RORγT) and cytokines (IL-17A and IL-17F), polymeric Ig receptor (pIgR), J-chain, and antimicrobial peptides defensin β1 (Defb1), Reg3γ, SAA1/SAA2, and SSA3. (B) Male mice showed similar trends, but lacked differences in RORγt, pIgR, and J-Chain.

FIGS. 4A-B show Early STAT: Ileal expression of helper T-cell regulatory transcription factors and cytokines in 4- and 8-week STAT and Control mice. Ileal expression of T-bet [Th1], GATA3 [Th2], Foxp3 [Treg], and RORγt [Th17] (A), and IFNγ [Th1], IL-4 [Th2], TGFβ1 [Treg], and IL-17 [Th17] (B) was quantitated by qPCR and normalized by GAPDH expression.

FIG. 5 shows decreased Th17 populations in 8-week STAT intestine identified by flow cytometric analysis. Lamina propria lymphocytes (LPL) from both small and large intestine were isolated from 8-week STAT mice (Control n=4, STAT n=4) and analyzed for intracellular, Th17-produced cytokines by flow cytometry. Plots gated on CD3+CD4+ LPL. IL-17⁺ and IL-22⁺ cells were decreased in STAT compared to control mice in both the small and large intestine.

FIG. 6 shows ileal expression of antimicrobial peptide genes in 4- and 8-week STAT. Ileal expression of defensin β1, Reg3γ, and Relmβ (4-week; Control n=4, STAT n=5, 8-week; Control n=3, STAT n=4) was quantitated by qPCR and normalized by GAPDH expression. While only Relmβ expression was significantly lower in STAT compared to control at 4 weeks (p<0.05), all three antimicrobial peptides examined were significantly lower in STAT at 8 weeks (p<0.05).

FIGS. 7A-C show the effect of STAT on ileal tissue structure. Atrophy was scored in H&E stained ileum samples from 4-week old control and STAT mice (n=5 each). (A-B) Example images from an ileal sample with an atrophy score of 0 (A) and 2 (B) are shown. (C) STAT mice showed significantly more ileal atrophy, * p<0.05 Mann-Whitney U.

FIG. 8 is a schematic of the process of ileal gene expression profiling by whole genome microarray in 8-week EarlySTAT and control mice.

FIGS. 9A-D show KEGG pathways, identified through Gene Set Enrichment Analysis (GSEA), that are down-regulated in STAT mice and involved in immunity. Genes from the ileal tissues from the mice indicated in FIGS. 1-5 that are down-regulated in STAT, and are involved in immunity, including (A) T cell receptors, (B) B cell receptors, (C) Toll like receptors, and (D) immune system processes. The figure is the representation of ileal tissues from seven mice: Control, n=3; STAT n=4.

FIGS. 10A-C show (A) a schematic of the process of transcriptional profiling analysis of intestinal tissue by Nanostring analysis; (B) the effects of STAT on expression of genes involved in intestinal immune responses; and (C) the predicted effects of STAT on biological functions. Male and female C57BL6J mice received sub-therapeutic antibiotic treatment (STAT) from birth, or not (control), n=4 each. In FIG. 10A, ileal tissue was collected at 8-weeks, at sacrifice, and RNA was isolated. Gene expression of 547 genes was quantified by the Nanostring Immunology Panel, and significant changes were detected by t-test. Alterations in biological functions were predicted using the Ingenuity Pathway Analysis. p<0.05 by Ingenuity Pathway Analysis, and z-score>|2| in at least one group.

FIG. 11 shows down-regulation of B cell markers and TLRs in 4- and 8-week STAT mice identified through both qPCR and microarray analyses. Ileal expression of the IgA J-chain, polymeric Ig receptor (pIgR), both related to polymeric IgA function, and TLR2 and TLR4 was quantitated by qPCR and normalized by GAPDH expression in 4- and 8-week STAT [4-week; Control n=3, STAT n=4, 8-week; Control n=3, STAT n=4].

FIGS. 12A-H show the effect of parents, diet and antibiotics on the abundance of SFB in fecal pellets. C57BL6J mice were bred, and 1 week prior to birth, 12 of the 17 mothers were exposed to sub-therapeutic antibiotic treatment (S, STAT), while 5 did not receive antibiotics (C, Control). Control offspring did not receive antibiotics; STAT offspring received STAT for 4, 8, or 28 weeks. All offspring were switched to a high fat diet at 6 weeks of age. Shown is SFB relative abundance in (A) parents, and (B-H) offspring at 3 to 8.5 weeks of age.

FIG. 13 shows study design for transmission of altered immune phenotype through microbiota transfer.

FIGS. 14A-K show transmission of altered immune phenotype through microbiota transfer. C57BL6J mice received sub-therapeutic antibiotic treatment (STAT; n=3), Control mice did not receive antibiotics (n=3). At 18 weeks, microbiota was transferred to germ-free Swiss-Webster mice (Control-microbiota recipients, n=7; STAT-microbiota recipients, n=8). Shown is ileal expression of Th17 cell transcription factor (RORγt), Th17 cytokines (IL17-A and IL-17F), and antimicrobial peptides in 18-week old control and STAT microbiota donor mice (A-E) and 8-week old control- and STAT-microbiota recipient mice (F-K). Expression levels were measured by qPCR and normalized by GAPDH expression.

FIGS. 15A-C show a schematic for transferring microbiota (A) and the results of transferring microbiota on gene expression levels (B) and predicted impacts on various biological functions (C). Microbiota from 3 control or STAT donors was transferred to 3-week old germ-free Swiss-Webster mice, and ileal tissue was collected at 8 weeks of age. (A) Ileal tissue was collected at 8-weeks, at sacrifice, and RNA was isolated. Gene expression of 547 genes was quantified by the Nanostring Immunology Panel, and significant changes were detected by t-test. Alterations in biologic functions were predicted using the Ingenuity Pathway Analysis. (B) Relative expression of genes significantly altered between STAT and control microbiota recipients. (C) Predicted biologic functions increased or decreased, p<0.05 by Ingenuity Pathway Analysis, and z-score>|2|.

FIGS. 16A-B show SFB prevalence in microbiota donor and recipient mice. Taxonomy was assigned based on the Green Genes May 2013 database, and the percentage of sequences classified as Candidatus Arthromitus, the candidate genus name for segmented filamentous bacteria (SFB), was calculated. SFB relative abundance in (A) the microbiota-donors, and (B), the microbiota-recipient mice, and each mouse is plotted.

FIGS. 17A-C show microbial correlations with immunologic markers. Germ-free Swiss Webster mice were colonized with microbiota from Control mice or STAT mice, as above. The intestinal microbiota was surveyed over time by high throughput sequencing at an average depth of approximately 5,800 sequences per sample. The Spearman correlation between (A) RORγT, (B) IL-17A, and (C) IL-17F ileal expression at 35 days-post transfer with the relative abundance of the predominant phylum over time was calculated. Phyla with at least one significant correlation (p<0.05) are shown.

FIGS. 18A-C show microbial correlation with immunologic markers at day 35. Germ-free Swiss Webster mice were colonized with microbiota from control mice or STAT mice. The intestinal microbiota was surveyed over time (1-34 days post transfer fecal specimens, 35 days post transfer cecal and ileal specimens) by high throughput sequencing at an average depth of approximately 5,800 sequences per sample. Taxonomic assignment was made through the QIIME pipeline using the May 20, 2013 Green Genes database of 16S microbial sequences. The Spearman correlation was calculated between (A) ileal RORγT, (B) SAA1/2, and (C) Relmβ expression at 35 days-post transfer with relative abundance of the predominant species (>1% in any sample). Microbiota with at least one significant correlation (p<0.05), and consistent correlation direction are shown. An ellipse with a forward slant represents a positive Spearman correlation, and a backwards slant represents a negative Spearman correlation, and the narrowness of the ellipse indicates the strength of the correlation (higher rho value). Significant correlations are indicated as * p<0.05, ** p<0.01. Microbiota names are reported at the lowest possible identifiable level, indicated by the letter preceding the underscore: o=order, f=family, g=genus, s=species.

FIGS. 19A-B are schematics of pulsed antibiotic treatment (PAT) schedule (A) and study groups for gene expression analysis (B). (A) Mice were exposed to 3 pulses of antibiotics (Amoxicillin or Tylosin), or no antibiotics, via breast milk or drinking water at the indicated time points. (B) Male mice, including 5 Control, 4 Amoxicillin, and 6 Tylosin, were fed a normal diet. They were sacrificed at 6 weeks of age and the terminal[1 cm] ileum without Peyer's Patches [TI-] was collected.

FIG. 20 shows ileal expression of helper T-cell regulatory transcription factors and their characteristic cytokines in 6-week PAT mice. Ileal expression of T-bet and IFN-γ [Th1], GATA3 and IL-4 [Th2], Foxp3, and TGFβ1 [Treg], and RORγt, IL-17A, and IL-17F [Th17] (Control n=5, Amoxicillin n=4, Tylosin n=6) was quantitated with qPCR and normalized by GAPDH expression.

FIG. 21 shows ileal expression of antimicrobial peptides in 6-week PAT and Control mice. Ileal expression of defensin β1, Reg3γ, and Relmβ (Control n=5, Amoxicillin n=4, Tylosin n=6) was quantitated by qPCR and normalized by GAPDH expression. Relmβ expression was significantly lower in Tylosin compared to control mice (p<0.05).

FIGS. 22A-B represent a schematic of the process of ileal gene expression profiling by whole genome microarray in 6-week and control mice. 1127 and 3065 reproducibly up- and down-regulated genes, respectively were identified from ˜45,000 genes of the mouse genome by T-test (p<0.05) and fold change (≧1.2 or ≦0.8) in 6-week PAT (Panel A; Control vs Amoxicillin and Panel B; Control vs Tylosin), respectively. DAVID analysis was performed using the regulated genes, and Gene Set Enrichment analysis (GSEA) was performed using whole gene data.

FIGS. 23A-C show KEGG pathways, identified through Gene Set Enrichment Analysis (GSEA), that are down-regulated in PAT and involved in immunity. (C, n=3), amoxicillin (A, n=3), and tylosin treated mice (T, n=3). Shown are pathways down-regulated in both 6-week PAT mouse groups, and are involved in immunity, including (A) T cell receptor pathway, (B) immune responses, and (C) Toll like receptor.

FIG. 24 shows down-regulation of B cell markers and TLRs in 6-week PAT, identified through qPCR and microarray. Ileal expression of J-chain, polymeric Ig receptor (pIgR), both related to polymeric IgA function, and TLR2 and TLR4 was quantitated by qPCR and normalized by GAPDH expression. Control n=5, Amoxicillin n=4, Tylosin n=6. Both qPCR and microarray data showed significantly down-regulated B cell markers with a downward trend in TLRs in PAT mice compared to controls.

FIG. 25 is a schematic of the early-life pulsed antibiotic treatment (EL-PAT) study. C57BL/6 mice were treated with either one (PAT1) or three (PAT3) therapeutic doses (50 mg/kg/day) of tylosin tartrate.

FIGS. 26A-B show decreased frequency of small intestine CD4⁺ IL-17A⁺ T-helper 17 (Th17) and CD4⁻ IL-17A⁺ lymphocytes in PAT mice. Small intestine lamina propria lymphocytes were isolated from 7-week old control and PAT mice. Intestinal CD4⁺ IL-17A⁺ and CD4− IL-17A populations were phenotyped based on surface and intracellular staining Cells were gated on CD45+ and either CD4+ or CD4− populations. Panel A: Cells with IL-17A expression were significantly decreased with one or three therapeutic doses of antibiotics in males. ***p<0.001, **p<0.01, Three antibiotic doses significantly decreased intestinal CD4⁺ IL-17A⁺ populations in females. (A)**p<0.01. Panel B: Additionally, three therapeutic doses of antibiotics decreased CD4⁻ IL-17A⁺ populations in females. *p<0.05.

FIG. 27 shows alpha rarefaction plots of phylogenetic diversity in fecal, cecal and ileal in Control, PAT1 and PAT3 treated male pups over time.

FIGS. 28A-B show alpha rarefaction plots of microbial community evenness and richness in ileal Control, PAT1 and PAT3 treatment groups at sacrifice. Ileal evenness (A) and richness (B) decreases with both one and three pulses of antibiotics at sacrifice, six-weeks and one week after the last antibiotic pulse in PAT1 and PAT3, respectively.

FIG. 29 depicts Principal Coordinate Analysis (PCoA) of fecal, ileal and cecal samples in male and female Control, PAT1 and PAT3 treatment groups. Plots were generated using weighted Unifrac distance metric. The three components explain 56%, 8% and 4.2% of the variance (total 68%). Both one and three therapeutic doses of Tylosin, early in life, significantly alter intestinal microbial populations in both males and females.

FIGS. 30A-E depict Principal Coordinate Analysis (PCoA) of ileal microbial communities and ileal T-cell populations in male and female Control, PAT1 and PAT3 treatment groups. Plots were generated using the Jenson-Shannon divergence (JSD) distance metric to determine the similarity between Control, PAT1 and PAT3. PAT1 and PAT3 ileal microbial communities are significantly different from Control and from each other (A, C, E, respectively) independent of sex (B). The frequency of ileal Th17 populations greater than 12.5% clusters with Control samples (A).

FIGS. 31A-C show the relative abundance of fecal, ileal and cecal microbial communities in dams and male and female offspring. Fecal samples were collected from nursing dams from the birth of pups to sacrifice. PAT Dams were treated with one therapeutic dose of Tylosin 5 days post-birth of offspring for 5 days to indirectly treat offspring (PAT1). Control microbial communities were transferred from Dams to pups and remain relatively stable over time, as shown in Panel A. One therapeutic dose of tylosin altered both the Dams' and offspring's microbial communities with blooms in Akkermansia muciniphila and Bacteroides uniformis and loss of Segemented Filamentous Bacteria, and remained altered in fecal, ileal and cecal samples seven weeks post antibiotic treatment, as depicted in Panel B. Further alterations in microbial communities occurred in the PAT3 group, which received two additional doses of the Tylosin antibiotic, including blooms in the family Enterobacteriaceae and Blautia producta, shown in Panel C.

FIGS. 32A-B depict the differential microbial abundance of ileal microbial communities and their association to Th17 population. Circular cladograms demonstrate significantly different taxa among treatment groups using the LEfSe module. Each color indicates the most abundant taxa in Control (light grey), PAT1, (darker grey) or PAT3 (darkest grey) groups. (A) Early-life PAT increases the relative abundance of Akkermansia muciniphila and decreases the abundance of S24-7, Candidatus Arthromitus (SFB) and Bifidobacterium. Th17 cells are positively associated with S24-7, Bifidobacterium and Candidatus Arthromitus (SFB) and negatively associated with Akkermansia muciniphila (B).

FIG. 33 is a schematic of NOD PAT/STAT study design. Pregnant NOD/ShiLTj mice were randomized into 3 groups: Control, STAT, & PAT. Control mice were maintained on non-acidified sterilized water. STAT pregnant dams were given low dose penicillin beginning late in pregnancy until pups were 12 weeks of age. PAT mice were given 3 therapeutic doses of tylosin starting on day 10 of life. At 6 weeks of age, 13 male (5 control, 3 STAT, 5 PAT) and 15 female (5 per group) NOD mice were sacrificed for immune-phenotyping by flow cytometry and ileal gene expression analysis by microarray and qPCR. From 10 weeks of age to 31 weeks of age, mice were tested for diabetes weekly by blood glucose measurement and urinary glucose detection. Onset of diabetes was defined as 2 consecutive weeks of blood glucose >250 mg/dl and glucosuria.

FIG. 34 shows type 1 diabetes incidence in female and male control, STAT, and PAT NOD mice. Diabetes onset was defined as 2 consecutive weeks with blood glucose >250 mg/dl and glucosuria. Diabetes incidence plots were compared using the log-rank test and the Gehan-Breslow-Wilcoxon test (# p<0.05, * p<0.04, respectively).

FIG. 35 shows pancreatic insulitis in 6-week old NOD mice. Upon necropsy, pancreata were preserved in modified Bouin's fixative, paraffin-embedded, and stained with aldehyde fuchsin, followed by a hemotoxylin and eosin counterstain. Five sections were made for each pancreas. Each of the sections is 5 microns thick with a 70 micron gap between sections. The most severely affected islet was scored from each section. Insulitis was scored on a scale of 0-4: 0, normal islet; 1, peri-insular leukocyte aggregates; 2, leukocyte infiltration <25%; 3, leukocyte infiltration >25%, but <75%; 4, leukocyte infiltration >75% and β-cell destruction, according to standard criteria. For the females, at least 24 islets per group (5 mice per group) were scored. STAT females had significantly more islet inflammation than control females. For the males, 25 islets were scored for the control and PAT group (5 mice per group), and 15 islets from the STAT group (3 mice). PAT males had significantly more insulitis than control or STAT males. The Kruskal-Wallis test with Dunn's multiple comparison correction was used to determine group differences, *p<0.05. C=control, S=STAT, and P=PAT.

FIG. 36 shows a decreased proportion of CD4+ FOXP3+ T regulatory (Treg) cells and CD4+RORγT+ (Th17) cells in the small intestinal lamina propria (SI-LP) of PAT-treated male NOD mice. Flow cytometry was performed on splenic and SI-LP cells from 6-week old male and female NOD mice. CD3 and CD4 surface stains and a live/dead stain were used to gate on live T-helper cells. Nuclear staining for FOXP3 and RORγT was performed to identify Treg and Th17 cells, respectively. By qPCR, Candidatus Arthromitus (SFB) was below detectable limits in fecal specimens collected at 3 weeks and 6 weeks of age from all of the mice used in the above analysis. The range for our SFB qPCR assay is from 10² to 10⁶ copies of SFB. Quantitation of Treg and Th17 cells isolated from the spleen and SI-LP. PAT decreased the percent of SI-LP Treg and Th17 cells in male, but not female NOD mice. C=controls, S=STAT, P=PAT. Statistical analysis was performed on each tissue separately, using the Kruskal-Wallis test followed by the Dunn's multiple comparison test, ** p≦0.01.

FIGS. 37A-B show that PAT significantly decreases SAA gene expression in the terminal ileum of NOD mice. RNA was extracted from the terminal ileum of 6-week old NOD mice. (A) SAA 1, 2, and 3 gene expression by qPCR in 6-week old NOD male mice. RNA was reverse transcribed into cDNA and then qPCR was performed for SAA1, 2, or 3 using an absolute quantitation method. Gene expression data were normalized to 18S rRNA expression. Statistical analysis was performed using the Kruskal-Wallis test, followed by Dunn's multiple comparison test, *p<0.05. The results for this experiment show consistent decreases in expression of the SAA genes in the ileum, detected by two independent methods (microarray (data in Table 3 and 4) and qPCR). (B) shows that ileal gene expression of SAA 1, 2, and 3 correlates positively with the proportions of Th17 or Treg cells in the small intestinal lamina propria in NOD mice. SAA 1, 2, and 3 ileal gene expression was determined by reverse transcriptase qPCR. The percent of Th17 and Treg cells was determined by flow cytometry. Spearman's correlation was used to determine whether there was a significant correlation between SAA 1, 2, and 3 gene expression and the percent of Th17 and Treg cells.

FIGS. 38A-B show that PAT decreases richness in fecal, cecal, and ileal samples from male and female NOD mice. Fecal samples from control, PAT-, and STAT-exposed male and female mice at 3, 6, 10, and 13 weeks of age, and cecal and ileal samples from 6-week old mice were studied. DNA was extracted and the V4 region of the 16S rRNA gene sequenced and analyzed. Samples (n=555) were rarefied to a depth of 4150, which only excluded 5 samples from the analysis. (A) Alpha rarefaction plots of observed OTUs. At all sample times, in all sample types, and in both males and females, OUT richness is lower in PAT-exposed mice compared to controls (FDR corrected p-value <0.05). However, after the final PAT pulse ending on day 39 of life, OTU richness partially recovered by 13 weeks of age. In males, STAT has significantly lower OUT richness than controls at 3 and 6 weeks of age, and in females, STAT has significantly lower richness than controls only at 3 weeks of age (FDR corrected p-value <0.05). In all other comparisons, there were no significant differences between STAT and controls, despite continued antibiotics until 13 weeks of age. Compared to fecal control samples, OTU richness was significantly higher for cecal control samples and lower for ileal samples (FDR corrected p-value <0.05). (B) Phylogenic diversity (PD). At all sample times, in all sample types, and both in males and females, PD is lower in PAT compared to controls (FDR corrected p-value <0.05). However, after the last PAT pulse, PD is partially recovered. Relationships with STAT, and for cecal and ileal samples are essentially as in (A) for OUT richness.

FIGS. 39 A-D show that PAT alters intestinal microbial community structure in male and female NOD mice. (A) Fecal microbiota. At all time points examined and in both males and females, PAT samples (medium grey circles) cluster distinctly from controls (black circles). Control and STAT samples essentially overlap at all time points. The difference with PAT is chiefly represented on PC1, which by definition accounts for the most diversity in this set of samples. From 3 to 13 weeks of age, the representation of all samples moves up on the PC2 axis and ultimately closer together on the PC1 axis, although PAT remains distinct. (B) Dynamic changes in the fecal microbiome. All time points of fecal samples displayed on a single set of axes. For (C) and (D), the Adonis (non-parametric multivariate analysis of variance) test in R was used to determine whether the separation between groups is statistically significant, *p<0.05, **p<0.01, ***p<0.001. (C) shows the effect of treatment on the grouping within the each sex. (D) shows the effect of sex within each treatment group. These results show that PAT samples have longterm impacts on microbial composition in both males and females. In addition, male and female fecal microbiota composition begins to differ starting at 6-weeks of age in controls

FIGS. 40A-C show cecal (A) and ileal (B) microbial community structure in 6-week old mice. PAT clusters distinctly from control and STAT. STAT has a partial effect in the males, with some samples clustering near controls and others near PAT. For (C), the Adonis test in R was used to determine whether the seperation between groups is statistically significant, *p<0.05, **p<0.01, ***p<0.001. As in fecal samples, PAT cecal and ileal microbial community composiiton is significantly different from controls.

FIG. 41 shows beta diversity as measured by Unifrac differs in PAT-treated NOD mice. Beta diversity, as measured by unweighted unifrac analysis of samples from control, STAT-, and PAT-treated mice. In male mice, intragroup beta diversity is similar from 3 to 13 weeks of life in control mice. In STAT-treated mice, intragroup beta diversity is similar to that of controls. STAT distance from controls is similar to control intragroup distance. PAT-treated mice have lower intragroup distance than controls. Distances from controls to PAT-treated mice is greater than control intragroup distance. Results in female mice are parallel to those for male mice.

FIGS. 42A-B show taxonomy of fecal, cecal, and ileal samples. 16S rRNA sequencing of the V4 region was performed on over 500 fecal, cecal, and ileal samples from male and female NOD mice. At the Phylum level (A), male and female control samples have similar compositions over time. Among the control cecal and ileal samples, males have less Verrucomicrobia than females. STAT fecal samples appear very similar to controls. In cecal and ileal samples from male STAT mice there is a bloom in Verrucomicrobia greater than in controls but less than in PAT. After the first antibiotic pulse, the PAT samples show a large bloom in Verrucomicrobia not seen in control mice, and decrease in Bacteroides. The PAT samples become more similar to controls over time but do not recover completely by 13 weeks (7 weeks after the last antibiotic exposure). Cecal and ileal samples follow the same trends as the fecal samples with some differences in the STAT males. At the species level (B), results parallel those observed at the phylum level. STAT male cecal and ileal samples have more Akkermansia muciniphila (a species within the Verrucomicrobia phylum) than controls, but less than that in PAT samples. PAT samples show a large bloom in A. muciniphila starting at 3 weeks (after the first antibiotic pulse); the proportions of A. muciniphila decrease over time but remain greater than that seen in controls.

FIGS. 43A-B show identification of taxa differences in NOD mice by group using LEfSe. From each sample, OTU relative abundances were analyzed using the LEfSe algorithm to identify differentially abundant taxa by treatment group or sex. The graphs shown are cladograms depicting these differences and their phylogenic relationships. The colors indicate that the taxon is significantly enriched. The size of the individual taxon circle correlates with the relative abundance of that taxon. (A) Comparison of male and female control NOD mice. Fecal microbial communities from male and female NOD mice were compared using LEfSe at weeks 3, 6, 10, and 13 of life. The darker grey shading indicates that the taxon is significantly enriched in males while the lighter grey shading indicates that the taxon is significantly enriched in females. At week 3 (pre-pubertal), the taxa in males and females were similar. At week 6, 10, and 13 of life, consistent taxa are over-represented in male mice. Furthermore, 6-week cecal and ileal samples from males show the same enriched taxa. Key taxa from the Actinobacteria phylum including the genus Bifidobacterium are consistently identified as enriched in males in our study before the development of diabetes. Since males are relatively protected from diabetes compared to females, we hypothesize that these may be key protective taxa and plan to follow up on these studies. (B) Comparison of male control and PAT mice. Similar comparisons were made in male mice either in the PAT or control group to determine the key differential taxa. The darker grey color indicates that the taxon is significantly enriched in control males while the lighter grey color indicates that the taxon is significantly enriched in PAT males. As in the male to female comparison, control male mice are relatively protected from diabetes development compared to PAT male mice, therefore taxa identified as enriched in male controls are hypothesized to be key protective taxa. Consistent with the male and female comparison data, we find that taxa from Actinobacteria phylum and more specifically the genus Bifidobacterium are enriched in male controls relative to PAT males. These differences are present in fecal samples at 6, 10, and 13 weeks of life as well as cecal and ileal samples from 6 weeks of life. At 3 weeks of life, the overall phylum of Actinobacteria is significantly greater in controls, but the genus Bifidobacterium does not reach statistical significance.

FIGS. 44A-C show that PAT decreases the relative abundance of Bifidobacterium genus and species in both females and males. The V4 region of the 16S rRNA gene was sequenced for 205 control and PAT samples from both male and female NOD mice. OTU picking and taxonomic assignment were performed using QIIME. Panel A. Relative abundance of Bifidobacterium genus. Each dot represents a sample, and the horizontal line represents the mean. Panel B. Relative abundance of 3 detectable Bifidobacterium species. The detectable named Bifidobacterium species were B. adolescentis, B. animalis, and B. pseudolongum. In both males and females, Bifidobacterium pseudolongum appears to be the dominant bifidobacterium species. In controls (black bars), Bifidobacteria especially B. pseudolongum are more highly abundant in males. In females Bifidobacteria are very infrequently seen in the ileum while in males they are abundant (light grey bar). In females PAT decreases the abundance of the 3 species of Bifidobacterium but there is some recovery by 13 weeks. STAT has increased abundance of all 3 of these species in females. In males, as with females, PAT decreases the abundance of these 3 species however there is very little recovery in males and no recovery in the ileal bifidobacterium. STAT appears similar to controls in fecal samples, with slightly reduced bifidobacteria species in the ileum. Panel C. Relative abundance of unnamed Bifidobacterium species. Bar height represents the mean and error bars depict the SEM. The unnamed Bifidobacterium species is present at a higher relative abundance in male controls up to nearly 6% compared with the named species, which have a relative abundance below 1%. Control male NOD mice have higher relative abundance of Bifidobacterium s than females in fecal samples (black bars) and ileal samples (light grey bars). Control male samples have higher relative abundance of Bifidobacterium s_ than PAT-treated mice, and the same trend is seen in females, although PAT females recover some Bifidobacterium s_{—} by the 13 weeks (last lighter grey bar).

FIG. 45 shows an overview of significant ileal genes in NOD mice among different treatment groups. Differential gene expression analysis was performed using the Empirical Bayes Method. Multiple testing adjustments were done by FDR correction with q<0.05. No genes were significantly differentially expressed between Control and STAT mice in either males or females, whereas between Control and STAT, there were >1000. In males, 69.3% of the differentially regulated genes were down in PAT, vs 42.1% in females.

FIG. 46 shows proportions of IFNγ-producing splenic CD4+ and CD8+ T-cells are increased in PAT-treated male NOD mice compared to controls. Leukocytes were isolated from spleens harvested from 6-week old control (n=7) and PAT-treated (n=7) NOD mice. Flow cytometric analysis was performed to assess T-cell cytokine production. Mann-Whitney U-test, ** p<0.01. Bar heights represent the means and error bars represent the standard deviations. FIG. 47 shows Taxa with significantly different abundances in control and PAT male NOD mice across timepoints and locations. Area under the curve analysis was used to determine significantly different taxa, with FDR correction for multiple comparisons. Taxa with at least two significant timepoints (indicated by *) are illustrated at the phylum, genus, and species level. Dark grey and light grey coloration indicates enrichment at the specified time point in control and in PAT specimens, respectively.

FIGS. 48A-E show the quantitation of Bifidobacterium in NOD mice by qPCR using Bifidobacterium genus-specific primers. Testing was performed on control and PAT male fecal samples at 4 timepoints: 3-, 6-, 10-, and 13-weeks of life. Panel A depicts the correlation of Bifidobacterium genus quantitation by qPCR and relative abundance by 16S rRNA sequencing. Spearman correlation was performed to determine statistical significance. Only samples with defined counts of Bifidobacterium by sequencing were used for this analysis. Panel B shows patterns of genus Bifidobacterium colonization over time. Panel C shows the quantitation of genus Bifidobacterium at 3-, 6-, 10-, and 13-weeks of life. The unpaired t-test was used to determine statistical significance * p<0.05, *** p<0.001. The median for each group was calculated and displayed as a line. Panel D shows Bifidobacterium levels in control mice by cage. Panel E shows that age of diabetes onset negatively correlates with Bifidobacterium copy number at 6-weeks of age. Spearman correlation was performed to determine statistical significance. Only samples from mice that developed diabetes were included in this analysis.

### DETAILED DESCRIPTION OF THE INVENTION

The present invention is based on an unexpected experimental observation in a mouse model that alteration in the composition of microbiota (e.g., as a result of antibiotic treatment), can produce short and long-term alterations in intestinal effector T-cell populations, B cells, innate immunity, and other effectors of innate and adaptive immunity, via modifiable changes in intestinal gene expression. Specifically, as demonstrated in the Examples section, below:
a) continuous exposure to low doses of antibiotics (modeled in sub-therapeutic antibiotic treatment (STAT) of mice at weaning or from birth) can be used to change immune effector T-cells, B cells, innate immunity, and the effector molecules they produce;
b) the antibiotic-altered (STAT) microbiota composition was sufficient to transfer the altered immune phenotypes to the recipient germ-free mice, which effect was unrelated to the presence of segmented filamentous bacteria (SFB), suggesting that other microbiota taxa are important for establishing the transferred phenotype;
c) early life low-dose antibiotics shape the immune responses, with only 4 weeks of exposure (in mice) sufficient for long-term or permanent changes in immune cell populations and gene expression in the gut;
d) pulsed antibiotic exposure (PAT) is sufficient to alter antibacterial peptide gene expression and can have large-scale long-lasting (to the time of sexual maturity) effects on gene expression in the terminal ileum; changes in gene expression induced by PAT exposures are highly similar to those due to STAT exposures;
e) PAT-induced changes in the microbiota trigger histological events in the ileum that correlate with decreased expression of SAA genes (SAA1, 2, 3) as well as changes in the expression of other genes (see Tables 3 and 4) and insulitis.
f) PAT-induced changes in the microbiota lead to changed bacterial compositions, changed ileal gene expression, changed T cell populations and the earlier onset of insulitis leading to type 1 diabetes (T1D).

This unexpected discovery highlights opportunities for prevention, diagnosis, and treatment of allergic, inflammatory and autoimmune diseases, including type 1 diabetes (T1D), using prebiotics, probiotics, synbiotics, antibiotics, or combinations thereof.

### DEFINITIONS

As used herein, the term “bacteria” encompasses both prokaryotic organisms and archaea present in mammalian microbiota.

The terms “intestinal microbiota”, “gut flora”, and “gastrointestinal microbiota” are used interchangeably to refer to bacteria in the digestive tract.

Specific changes in microbiota discussed herein can be detected using various methods, including without limitation quantitative PCR or high-throughput sequencing methods which detect over- and under-represented genes in the total bacterial population (e.g., 454-sequencing for community analysis; screening of microbial 16S ribosomal RNAs (16S rRNA), etc.), or transcriptomic or proteomic studies that identify lost or gained microbial transcripts or proteins within total bacterial populations. See, e.g., U.S. Patent Publication No. 2010/0074872; Eckburg et al., Science, 2005, 308:1635-8; Costello et al., Science, 2009, 326:1694-7; Orrice et al., Science, 2009, 324:1190-2; Li et al., Nature, 2010, 464: 59-65; Bjursell et al., Journal of Biological Chemistry, 2006, 281:36269-36279; Mahowald et al., PNAS, 2009, 14:5859-5864; Wikoff et al., PNAS, 2009, 10:3698-3703.

As used herein, the term “probiotic” refers to a substantially pure bacteria (i.e., a single isolate, live or killed), or a mixture of desired bacteria, or bacterial extract, and may also include any additional components that can be administered to a mammal. Such compositions are also referred to herein as a “bacterial inoculant.” Probiotics or bacterial inoculant compositions of the invention are preferably administered with a buffering agent (e.g., to allow the bacteria to survive in the acidic environment of the stomach and to grow in the intestinal environment). Non-limiting examples of useful buffering agents include saline, sodium bicarbonate, milk, yogurt, infant formula, and other dairy products.

As used herein, the term “prebiotic” refers to an agent that increases the number and/or activity of one or more desired bacteria. Non-limiting examples of prebiotics useful in the methods of the present invention include fructooligosaccharides (e.g., oligofructose, inulin, inulin-type fructans), galactooligosaccharides, N-acetylglucosamine, N-acetylgalactosamine, glucose, other five- and six-carbon sugars (such as arabinose, maltose, lactose, sucrose, cellobiose, etc.), amino acids, alcohols, resistant starch (RS), and mixtures thereof. See, e.g., Ramirez-Farias et al., Br J Nutr (2008) 4:1-10; Pool-Zobel and Sauer, J Nutr (2007), 137:2580S-2584S.

The term “synbiotic” refers to a combination of a probiotic and a prebiotic.

As used herein, the term “metagenome” refers to genomic material obtained directly from a subject, instead of from culture. Metagenome is thus composed of microbial and host components.

The terms “treat” or “treatment” of a state, disorder or condition include:
(1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or
(2) inhibiting the state, disorder or condition, i.e., arresting, reducing or delaying the development of the disease or a relapse thereof (in case of maintenance treatment) or at least one clinical or sub-clinical symptom thereof; or
(3) relieving the disease, i.e., causing regression of the state, disorder or condition or at least one of its clinical or sub-clinical symptoms.

The benefit to a subject to be treated is either statistically significant or at least perceptible to the patient or to the physician.

As used herein in connection with administration of antibiotics, the term “antibiotic treatment” comprises antibiotic exposure.

As used herein, the term “early in life” refers to the period in life of a mammal before growth and development is complete. In case of humans, this term refers to pre-puberty, preferably within the first 6 years of life.

A “therapeutically effective amount” means the amount of a bacterial inoculant or a compound (e.g., an antibiotic or a prebiotic) that, when administered to a subject for treating a state, disorder or condition, is sufficient to effect such treatment. The “therapeutically effective amount” will vary depending on the compound, bacteria or analogue administered as well as the disease and its severity and the age, weight, physical condition and responsiveness of the mammal to be treated.

When used in connection with antibiotic administration, the term “therapeutic dose” refers to an amount of an antibiotic that will achieve blood and tissue levels corresponding to the minimal inhibitory concentration (MIC) for at least 50% of the targeted microbes, when used in a standardized in vitro assay of susceptibility (e.g., agar dilution MICs; see Manual of Clinical Microbiology, ASM Press).

The term “sub-therapeutic antibiotic treatment” or “sub-therapeutic antibiotic dose” refers to administration of an amount of an antibiotic that will achieve blood and tissue levels below the minimal inhibitory concentration (MIC) for 10% of targeted organisms, when used in a standardized in vitro assay of susceptibility (e.g., agar dilution MICs; see Manual of Clinical Microbiology, ASM Press). Non-limiting examples of useful doses for sub-therapeutic antibiotic treatment include 1-5 mg/kg/day.

As used herein, the phrase “pharmaceutically acceptable” refers to molecular entities and compositions that are generally regarded as physiologically tolerable.

As used herein, the term “combination” of a bacterial inoculant, probiotic, analogue, or prebiotic compound and at least a second pharmaceutically active ingredient means at least two, but any desired combination of compounds can be delivered simultaneously or sequentially (e.g., within a 24 hour period).

“Patient” or “subject” as used herein refers to mammals and includes, without limitation, human and veterinary animals.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “relative abundance” refers to the abundance of a specific taxon normalized to the total eubacteria.

The term “control microbiota” as used herein in connection with the diagnostic methods of the invention refers to microbiota from a healthy subject (or average of several healthy subjects) of the same gender and ethnicity, and of a similar age (±5 years), and preferably (but not necessarily) residing in the same geographic region as the subject who is being diagnosed. According to the invention, control microbiota can be either obtained and/or assayed in the same assay as the test microbiota or can be used as a standard predetermined value.

### Methods of the Invention

a. Methods Involving Administration of Probiotics and Prebiotics

Bacterial strains administered according to the methods of the present invention can comprise live bacteria. One or several different bacterial inoculants can be administered simultaneously or sequentially (including administering at different times). Such bacteria can be isolated from microbiota and grown in culture using known techniques. However, many bacterial species are very difficult to culture and administration of others may lead to various undesirable side-effects. The present invention therefore comprises administering “bacterial analogues”, such as recombinant carrier strains expressing one or more heterologous genes derived from the bacteria affected in a disease. The use of such recombinant bacteria may allow the use of lower therapeutic amounts due to higher protein expression and may simultaneously allow to avoid any potential harmful side-effects associated with reintroduction of specific bacterial strains. Non-limiting examples of recombinant carrier strains useful in the methods of the present invention include E. coli and Lactobacillus (e.g., E. coli and Lactobacillus expressing cag island-encoded type IV secretion system of H. pylori). Methods describing the use of bacteria for heterologous protein delivery are described, e.g., in U.S. Pat. No. 6,803,231.

In certain embodiments, killed bacterial cells and bacterial cell extracts can be utilized as the probiotics of the invention (see, e.g., Round et al., Proc. Natl. Acad. Sci. USA, 2010, 107: 12204)

In certain embodiments, a conditionally lethal bacterial strain can be utilized as the inoculant or to deliver a recombinant construct. Such a conditionally lethal bacteria survives for a limited time typically when provided certain nutritional supplements. It is contemplated that such a supplement could be a liquid, formulated to contain the nutritional component necessary to keep the bacteria alive. It is further contemplated that a patient/subject would drink such a supplement in intervals to keep the bacteria alive. Once the supplement is depleted, the conditional lethal bacteria die. Methods relating to conditionally lethal strains of H. pylori are described in U.S. Pat. No. 6,570,004.

In certain embodiments, the bacterial inoculant used in the methods of the invention further comprises a buffering agent. Examples of useful buffering agents include saline, sodium bicarbonate, milk, yogurt, infant formula, and other dairy products.

In certain embodiments, the bacterial inoculant is administered in combination with one or more prebiotics which promote growth and/or immunomodulatory activity of the bacteria in the inoculant. Non-limiting examples of prebiotic agents useful in the methods of the present invention include fructooligosaccharides (e.g., oligofructose, inulin, inulin-type fructans), galactooligosaccharides, N-acetylglucosamine, N-acetylgalactosamine, glucose, other five and six-carbon sugars (e.g., arabinose, maltose, lactose, sucrose, cellobiose, etc.), amino acids, alcohols, resistant starch (RS), and mixtures thereof. Additional prebiotic agents can be selected based on the knowledge of a particular bacteria and/or immunological response implicated in a disease to be treated.

Administration of a bacterial inoculant can be accomplished by any method likely to introduce the organisms into the desired location. Non-limiting examples of useful routes of delivery include oral, rectal, fecal (by enema), and via naso/oro-gastric gavage.

Bacteria can be mixed with a carrier and (for easier delivery to the digestive tract) applied to liquid or solid food, or feed or to drinking water. The carrier material should be non-toxic to the bacteria and the subject/patient. Non-limiting examples of probiotic formulations useful in the methods of the present invention include oral capsules and saline suspensions for use in feeding tubes, transmission via nasogastric tube, or enema. If live bacteria are used, the carrier should preferably contain an ingredient that promotes viability of the bacteria during storage. The formulation can include added ingredients to improve palatability, improve shelf-life, impart nutritional benefits, and the like. If a reproducible and measured dose is desired, the bacteria can be administered by a rumen cannula.

The dosage of the bacterial inoculant or compound of the invention will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve colonization of the gastrointestinal tract with the desired bacterial inoculant, e.g. 10⁶, 10⁷, 10⁸, 10⁹, and 10¹⁰ CFU for example, can be administered in a single dose. Lower doses can also be effective, e.g., 10⁴, and 10⁵ CFU.

One of the organisms contemplated for administration to restore the gastrointestinal microbiota is Helicobacter pylori. H. pylori is Gram-negative, microaerophilic, fastidious bacterium that colonizes specifically the surface of the mucosa of the stomach. Non-limiting examples of H. pylori strains useful in the methods of the invention include live or conditionally lethal cagA positive (cagA+) strains (i.e., strains possessing a full functioning cag island-encoded type IV secretion system that can inject the CagA protein and other H. pylori constituents into epithelial cell), live or conditionally lethal cagA negative (cagA-) strains, as well as live or conditionally lethal strains varying in VacA activity (of genotypes s1 or s2, m1 or m2, i1 or i2) and/or in expression of the type I or type II Lewis antigen pathways. In certain embodiments, the bacteria administered in the therapeutic methods of the invention comprise H. pylori and one or more additional bacterial strains (such as, e.g., Oxalobacter species, Lactobacillus species, etc.). In one embodiment, the invention provides a method for treating asthma, allergy, gastrointestinal reflux disease (GERD), eosinophilic esophagitis, and related disorders in a mammal comprising administering to the mammal a therapeutically effective amount of H. pylori live or conditionally lethal cagA positive (cagA+) strain.

In a separate embodiment, the invention comprises the use of prebiotics that stimulate the correlated taxa. For example, as specified in the Examples section below, genera Bifidobacter and Odoribacter were positively associated with SAA expression. The growth or activity of these genera could be stimulated by including maltose, lactose and/or sucrose in the diet (to preferentially select for Bifidobacter species) or Arabinose (to select for Odoribacter species). Conversely, since Blautia was inversely associated with SAA, then to drive down SAA, including cellobiose in the diet would be a means to preferentially select for organisms in the genus Blautia. Since organisms in the genus Ruminococcus were positively associated with RoRGT, then inclusion of lactose in the diet would select for those organisms.

b. Methods Involving Administration of Antibiotics

Non-limiting examples of antibiotics useful in the methods of the invention include beta-lactams (e.g., Penicillin VK, Penicillin G, Amoxicillin trihydrate), nitroimidazoles, macrolides (e.g., Tylosin tartrate, Erythromycin, Azithromycin, and Clarithromycin), tetracyclines, and glycopeptides (e.g., Vancomycin). In one specific embodiment, the method comprises administering Penicillin VK or Penicillin G at 1 mg/kg body weight per day for at least four weeks of life. In another specific embodiment, the method comprises administering Amoxicillin trihydrate at 25 mg/kg body weight per day for 1 to 3 treatments each lasting 3 to 5 days. In yet another specific embodiment, the method comprises administering Tylosin tartrate at 50 mg/kg body weight per day for 1 to 3 treatments each lasting 3 to 5 days.

### Pharmaceutical Compositions

While it is possible to use a bacterial inoculant or compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Although there are no physical limitations to delivery of the formulations of the present invention, oral delivery is preferred for delivery to the digestive tract because of its ease and convenience, and because oral formulations readily accommodate additional mixtures, such as milk, yogurt, and infant formula. Bacteria can be also administered via naso/orogastric gavage, rectally, or via fecal route (by enema).

### Combination Treatments

For an enhanced therapeutic effect, the bacterial inoculants or compounds as described herein can be administered in combination with other therapeutic agents or regimes as discussed. The choice of therapeutic agents that can be co-administered with the bacterial inoculants or compounds of the invention depends, in part, on the condition being treated.

Non-limiting examples of additional pharmaceutically active compounds useful for treatment of rheumatoid arthritis, psoriasis, and related disorders include anti-inflammatory agents, cytokines, and anti-TNF-monoclonal antibodies.

Non-limiting examples of diabetes drugs useful in the combination therapies of the present invention include insulin, proinsulin, insulin analogs, activin, glucagon, somatostatin, amylin, actos (pioglitazone), amaryl (glimepiride), glipizide, avandia (rosiglitazone), glucophage, glucotrol, glucovance (a combination of glyburide and metformin), and the like. See, e.g., U.S. Pat. No. 6,610,272. The term “insulin” encompasses natural extracted human insulin, recombinantly produced human insulin, insulin extracted from bovine and/or porcine sources, recombinantly produced porcine and bovine insulin and mixtures of any of these insulin products. In accordance with the present invention, administering a bacterial inoculant or compound of the present invention in combination with insulin is expected to lower the dose of insulin required to manage the diabetic patient, while also alleviating the symptoms of metabolic syndrome.

In accordance with the present invention there may be numerous tools and techniques within the skill of the art, such as those commonly used in molecular immunology, cellular immunology, pharmacology, and microbiology. Such tools and techniques are describe in detail in e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Use of Sub-Therapeutic Antibiotic Treatment (STAT) to Affect Immunity

This example demonstrates that the use of low-dose antibiotics, using penicillin as a model, can change microbiome compositions, which then change immune cell populations and effector molecules in the gut. All experiments were performed in mice, which were housed on a 12-hour light dark cycle, and all procedures were carried out under IACUC approved protocols.

TimeSTAT (demonstrates that initiating sub-therapeutic antibiotic treatment (STAT) at weaning or from birth can influence immune responses). C57BL/6J (Jackson Labs, Bar Harbor, Me.) mice either received no antibiotics (control, females n=5, males n=5) or continuous STAT penicillin starting at birth (STATb, females n=4, males n=5), or at weaning at 4 weeks (STATw, females n=5, males n=5). Mice were weaned at 4 weeks onto normal chow (13.2% fat, 5053 PicoLab Rodent Diet 20, LabDiet, Brentwood, Mo.).

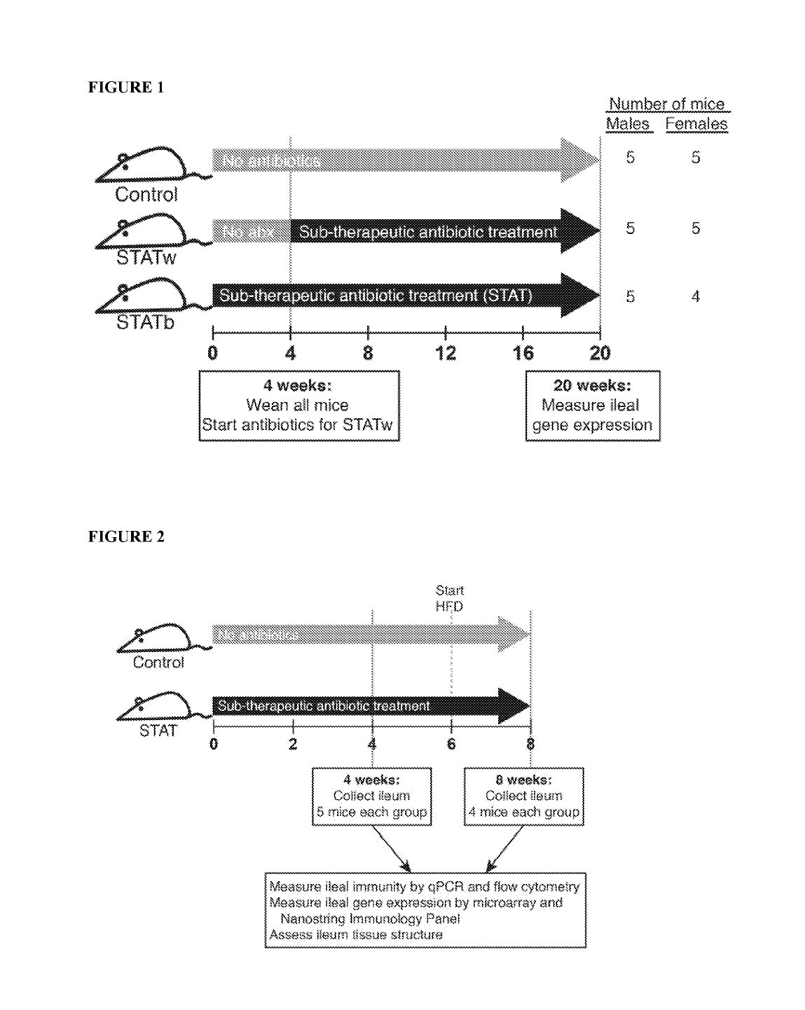

FIG. 1 shows a schematic of TimeSTAT study design. C57B16J mice received sub-therapeutic antibiotic treatment (STAT) beginning from weaning 4-weeks of age (STATw, n=5 females, 5 males), or from birth (STATb, n=4 females, 5 males), control mice did not receive antibiotics (n=5 females, 5 males). Both STATw and STATb remained on lifelong STAT after initial exposure. Mice were sacrificed at 20 weeks of age. The terminal[1 cm] ileum without Peyer's Patches [TI-] was collected for gene expression analysis by qPCR.

EarlySTAT (demonstrates that early life low-dose antibiotics shapes the immune responses, with only 4 weeks of exposure sufficient to change immune cell populations and gene expression in the gut). C57BL/6J (Jackson Labs, Bar Harbor, Me.) mice either received no antibiotics (control) or continuous STAT penicillin. Mice were weaned at 4 weeks onto normal chow (13.2% fat, 5053 PicoLab Rodent Diet 20, LabDiet, Brentwood, Mo.), then switched to a high fat diet (45% kcal from fat, D12451, Research Diets, New Brunswick, N.J.) at 6 weeks of life. Mice were sacrificed at 4, 8, and 18 weeks of age. The terminal[1 cm] ileum without Peyer's Patches [TI-] was collected for gene expression analysis by qPCR. The proximal 1 cm of ileum was used for histological analysis of tissue structure, and the remaining ileum was used to determine immunity phenotypes by flow cytometry.

FIG. 2 shows a schematic of EarlySTAT study design and study groups for gene expression analysis. C57BL6J mice were exposed to sub-therapeutic antibiotic treatment (STAT) with penicillin via their drinking water from birth, or no antibiotics (Control). Mice were fed a normal diet until 6 weeks of age, then they were fed high fat diet. 5 mice from each experimental group were sacrificed at 4 weeks, and 4 mice from each group were sacrificed at 8 weeks. The terminal[1 cm] ileum without Peyer's Patches [TI-] was collected for gene expression analysis by qPCR. The proximal 1 cm of ileum was used for histological analysis of tissue structure, and the remaining ileum was used to determine immunity phenotypes by flow cytometry. Ileal gene expression was also measured by microarray and the nCounter GX Mouse Immunology Kit (Nanostring Technologies, Seattle, Wash., USA).

Quantitative PCR (qPCR).

Total RNA was extracted from mouse tissues using the RNeasy Mini Kit (Qiagen), according to the manufacturer's instructions. Total RNA was reverse transcribed to cDNA using the Verso™ cDNA Synthesis Kit (Thermo Scientific). To generate standards for expression analysis of each target gene, the cDNA region of interest was PCR-amplified and the PCR product was cloned into pGEM-T easy (Promega). qPCR was performed with LightCycler 480 SYBR Green I Master mix (Roche) and run in a LightCycler 480 system (Roche). Target mRNA was normalized to GAPDH mRNA as an internal control in each sample. The primer sequences for qPCR are shown in Table 1.

TABLE 1
Primer sequences for T-cell and cytokine qPCRs
(SEQ ID NOS: 1-38, consecutively from top to bottom)

| | | | | | Product | Product |
|---|---|---|---|---|---|---|
| | | | | Tm | from | from |
| Gene | Orient | Sequence (5′→3′) | Location | (° C.) | gDNA | RNA |
| T-bet | F | CAA CAA CCC CTT TGC CAA AG | Exon 5, 1097-1116 | 66.3 | 501 | 109 |
| | R | TCC CCC AAG CAG TTG ACA GT | Exon 6, 1186-1205 | 66.9 | | |
| GATA-3 | F | AGA ACC GGC CCC TTA TGA A | Exon 3, 1074-1095 | 65.4 | 5613 | 72 |
| | R | ACT TCG CCC ACC ATC TCC | Exon 4, 1128-1145 | 66.5 | | |
| Foxp3 | F | CAC CCA GCA AAG ACA GCA ACC | Exon 7, 782-802 | 67.9 | 3443 | 314 |
| | R | GCA AGA GCT CTT GTC CAT TGA | Exon 10, 1075-1095 | 64.4 | | |
| RORγt | F | CCG CTG AGA GGG CTT CAC | Exon 1, 75-92 | 66.3 | 12287 | 230 |
| | R | TGC AGG AGT AGG CCA CAT TAC A | Exon 3, 282-304 | 66.5 | | |
| IFN-γ | F | GAT GCA TTC ATG AGT ATT GCC AAG T | Exon 3, 439-463 | 66.7 | 2446 | 87 |
| | R | GTG GAC CAC TCG GAT GAG CTC | Exon 4, 505-525 | 68.4 | | |
| IL-4 | F | TCG GCA TTT TGA ACG AGC TC | Exon 1, 188-207 | 67.2 | 4636 | 216 |
| | R | GAA AAG CCC GAA AGA GTC TC | Exon 3, 384-403 | 61.8 | | |
| TGF-β1 | F | ATG ACA TGA ACC GAC CCT TC | Exon 5, 1619-1638 | 63.7 | 7801 | 288 |
| | R | TGT GTT GGT TGT AGA GGG CA | Exon 7, 1887-1906 | 64.2 | | |
| 1L-10 | F | ATG CCT GGC TCA GAC | Exon 1, 76-88 | 60.2 | 1107 | 219 |
| | R | GTC CTG CAT TAA GGA GTC G | Exon 2, 276-294 | 59.8 | | |
| 1L-17A | F | CAG CAG CGA TCA TCC CTC AAA G | Exon 2, 131-152 | 70.0 | 1617 | 302 |
| | R | CAG GAC CAG GAT CTC TTG CTG | Exon 3, 412-432 | 66.1 | | |
| IL-17F | F | GAG GAT AAC ACT GTG AGA GTT GAC | Exon 2, 216-239 | 60.6 | 1496 | 209 |
| | R | GAG TTC ATG GTG CTG TCT TCC | Exon 3, 404-424 | 63.6 | | |
| 1L-22 | F | CAT GCA GGA GGT GGT ACC TT | Exon 4, 395-414 | 63.9 | 3779 | 198 |
| | R | CAG ACG CAA GCA TTT CTC AG | Exon 6, 573-592 | 63.7 | | |
| β defensin 1 | F | AGG TGT TGG CAT TCT CAC AAG | Exon 1, 113-133 | 64.0 | 17847 | 131 |
| | R | GCT TAT CAT GTT TAC AGG TTC CC | Exon 2, 221-243 | 63.5 | | |
| Reg3γ | F | TCA GGT GCA AGG TGA AGT TG | Exon 2, 98-137 | 63.9 | 987 | 233 |
| | R | GGC CAC TGT TAC CAC TGC TT | Exon 4, 318-330 | 64.0 | | |
| Relmβ | F | AAG GAA GCT TCT AGT CGT CAA | Exon 2-3, 347-370 | 63.1 | 1564 | 289 |
| | R | GAG TCA GGT TTC CTG GTC GAG | Exon 4, 615-635 | 65.1 | | |
| J-chain | F | GAA CTT TGT ATA CCA TTT GTC | Exon 2, 358-383 | 63.4 | 1054 | 88 |
| | | AGA CG | | | | |
| | R | CTG GGT GGC AGT AAC AAC CT | Exon 3-4, 426-445 | 63.9 | | |
| p1gR | F | AGT AAC CGA GGC CTG TCC TT | Exon 3, 523-542 | 63.8 | 2660 | 67 |
| | R | GTC ACT CGG CAA CTC AGG A | Exon 4, 570-589 | 64.3 | | |
| TLR2 | F | CAT CAC CGG TCA GAA AAC AA | Exon 3, 295-334 | 63.6 | 2370 | 271 |
| | R | GTC ACC ATG GCC AAT GTA GG | Exon 4, 546-565 | 65.2 | | |
| TLR4 | F | TCA GAA CTT CAG TGG CTG GA | Exon 2, 250-269 | 63.7 | 5361 | 188 |
| | R | GAG GCC AAT TTT GTC TCC AC | Exon 3, 418-437 | 63.4 | | |
| GAPDH | F | TGG TGA AGG TCG GTG TGA AC | Exon 3, 52-71 | 66.1 | 1858 | 123 |
| | R | CCA TGT AGT TGA GGT CAA TGA AGG | Exon 3, 151-174 | 65.4 | | |

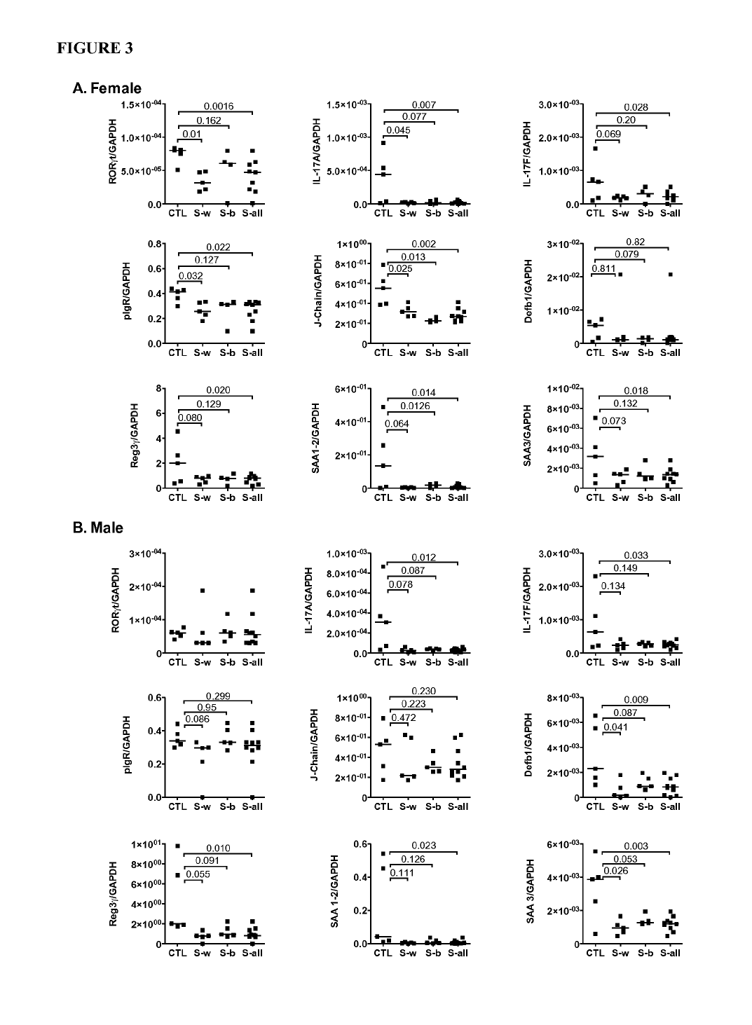

C57B16J mice received sub-therapeutic antibiotic treatment (STAT) beginning at 4-weeks of age (weaning, STATw, n=5 females, 5 males), or at birth (STATb, n=4 females, 5 males), control mice did not receive antibiotics (n=5 females, 5 males). Both STATw and STATb remained on lifelong STAT after initial exposure. Expression of immunological markers in ileal tissue was measured by qPCP at 20 weeks of age in both female and male mice. In female mice, there were significant reductions in Th17 transcription factor (RORγT) and cytokines (TH-17A and T111.7F), polymeric Ig receptor (pIgR), J-chain, and antimicrobial peptides defensin β1 (Defb1), Reg3γ, SAA1/SAA2, and SSA3 (FIG. 3A). Male mice showed similar trends, but lacked differences in RORγt, pIgR, and J-Chain (FIG. 3B).

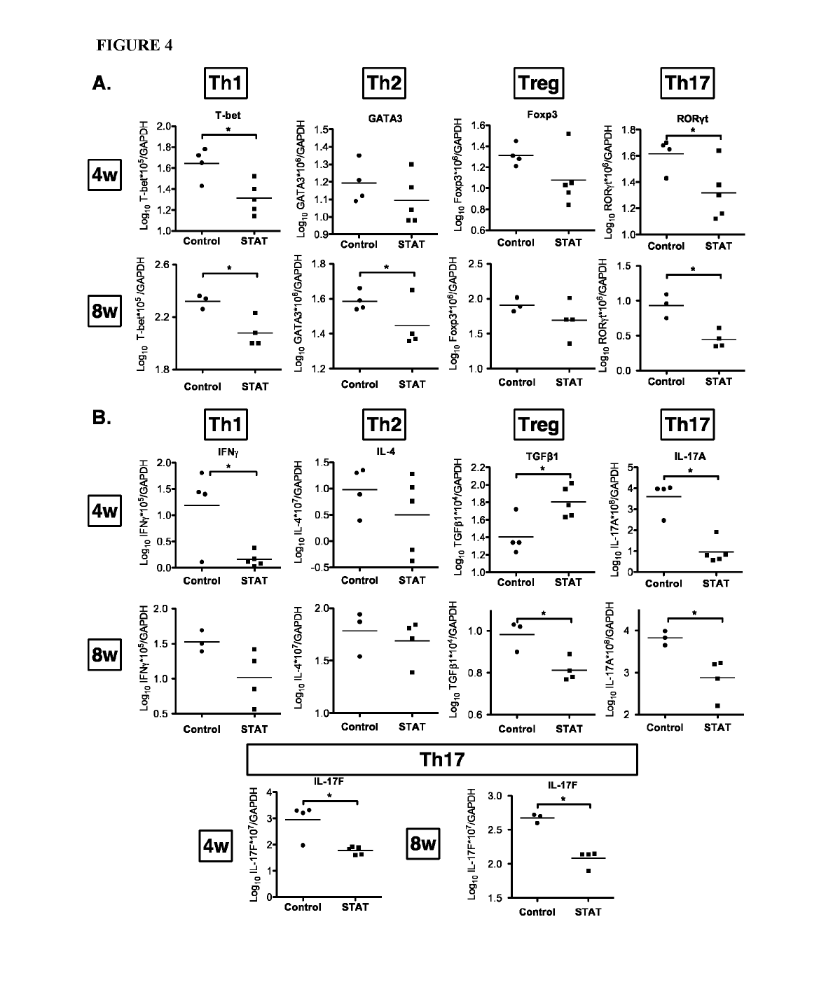

Ileal expression of T-bet [Th1], GATA3 [Th2], Foxp3 [Treg], and RORγt [Th17] (FIG. 4A), and IFNγ [Th1], IL-4 [Th2], TGFβ1 [Treg], and IL-17 [Th17] (FIG. 4B) (4-week; Control, n=4, STAT, n=5, 8-week; Control, n=3, STAT, n=4) was quantitated by qPCR and normalized by GAPDH expression. Expression of all four transcription factors trended downwards in STAT compared to control mice. There was significantly down-regulated expression of T-bet (at 4- and 8-weeks), GATA3 (at 8-weeks), and RORγt (at 4- and 8-weeks) in STAT (p<0.05). There was also significantly down-regulated expression of IFNγ (at 4-weeks), TGFβ1 (at 8-weeks), and IL-17 (at 4- and 8-weeks) in STAT (p<0.05). These studies provide evidence that low dose antibiotic exposure can be used to change immune effector T-cell populations and the effector molecules they produce.

Lamina propria lymphocytes (LPL) from both small and large intestine were isolated from 8-week STAT mice (Control n=4, STAT n=4) and analyzed for intracellular, Th17-produced cytokines by flow cytometry. Plots gated on CD3+CD4+LPL. IL-17⁺ and IL-22⁺ cells were decreased in STAT compared to control mice in both the small and large intestine (FIG. 5).

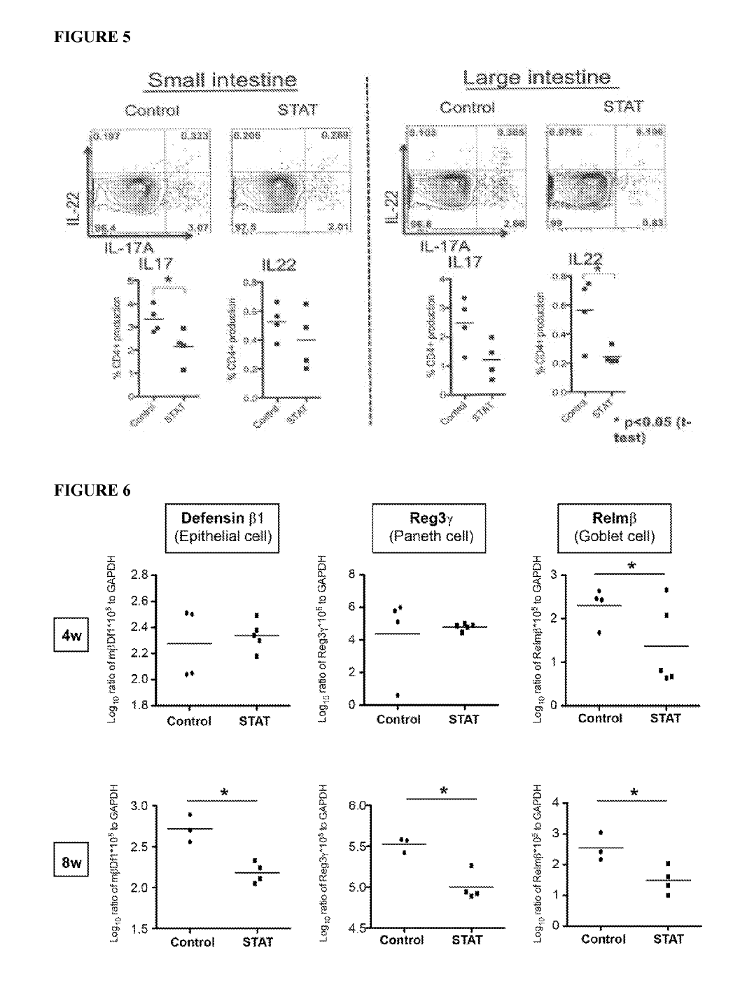

Ileal expression of defensin β1, Reg3γ, and Relmβ (4-week; Control n=4, STAT n=5, 8-week; Control n=3, STAT n=4) was quantitated by qPCR and normalized by GAPDH expression. While only Relmβ expression was significantly lower in STAT compared to control at 4 weeks (p<0.05), all three antimicrobial peptides examined were significantly lower in STAT at 8 weeks (p<0.05) (FIG. 6). These findings provide examples of how the STAT exposure changed intestinal expression of antimicrobial peptide genes.

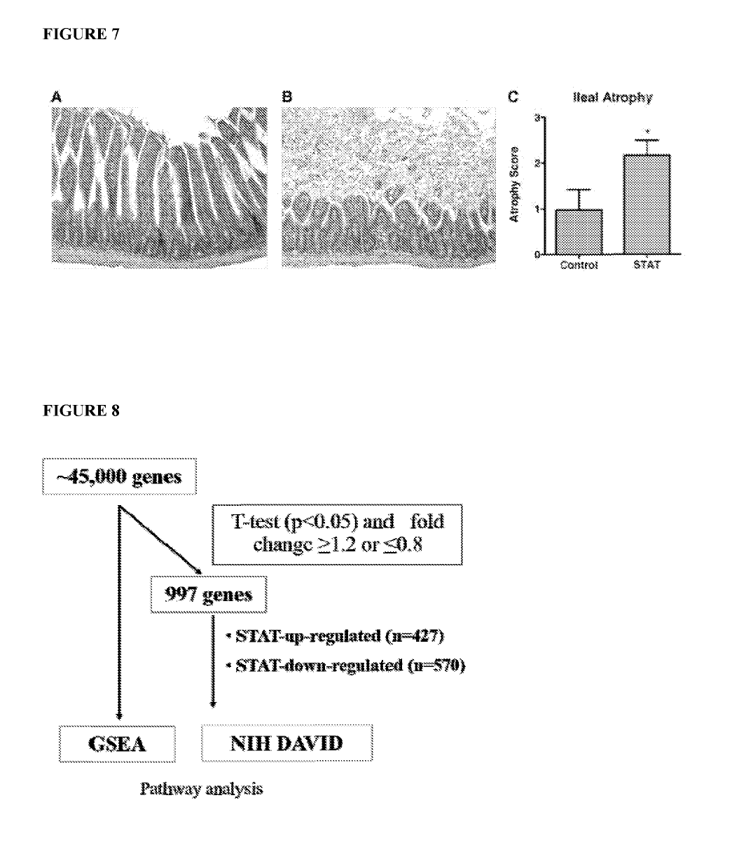

Atrophy was scored in hematoxylin and eosin (H&E) stained ileum samples from 4-week old control and STAT mice (n=5 each). STAT mice showed significantly more ileal atrophy (FIG. 7, * p<0.05 Mann-Whitney U).

Gene Expression Profiling.

Ileal gene expression profiling of STAT, PAT, and control animal groups, was performed using the Affymetrix Genechip system (Affymetrix). Total RNA quality and quantity were determined using the Agilent 2100 Bioanalyser and Nanodrop ND-1000. Total RNA (100 ng) was used to prepare cDNA following the Affymetrix 39IVT Express Kit labelling protocol (Affymetrix). Standardized array processing procedures recommended by Affymetrix were performed, including hybridization, fluidics processing and scanning of the Affymetrix MG-430 2.0 arrays. GeneSpring GX11 software (Agilent Technologies) was used to normalize the raw data (Affymetrix CEL files) by Robust Multichip Average algorithm (RMA). Gene set enrichment analysis (GSEA) was used to identify significantly enriched gene expression patterns underlying fatty acid and lipid metabolism, by querying the C2 (curated pathways) and C5 (Gene Ontologies) categories of the GSEA MolSig v.3 database.

Gene Expression Profiling Via Nanostring Immunology Panel.

Male and female C57BL6J mice received sub-therapeutic antibiotic treatment (STAT, n=4 each) from birth, or did not receive antibiotics (control, n=4 each) (FIG. 2). RNA was extracted from ileal tissue using the miRNeasy mini kit (Qiagen, Redwood City, Calif., USA) at 8 weeks of age and the expression levels of 547 genes related to immunology were measured by nCounter GX Mouse Immunology Kit (Nanostring Technologies, Seattle, Wash., USA). Significant differences between treatment and control were determined by t-test. For functional characterization, the significant changes were analyzed by Ingenuity Pathway Analysis (Qiagen, Redwood City, Calif.).

997 reproducibly regulated genes were identified from ˜45,000 genes of the mouse genome by T-test (p<0.05) and fold change (≧1.2 or ≦0.8) in 8-week STAT mice (FIG. 8). DAVID analysis was performed using the regulated genes, and Gene Set Enrichment analysis (GSEA) was performed using whole gene data. These data provide examples of how STAT exposure has widespread effects on gene expression in the ileum.

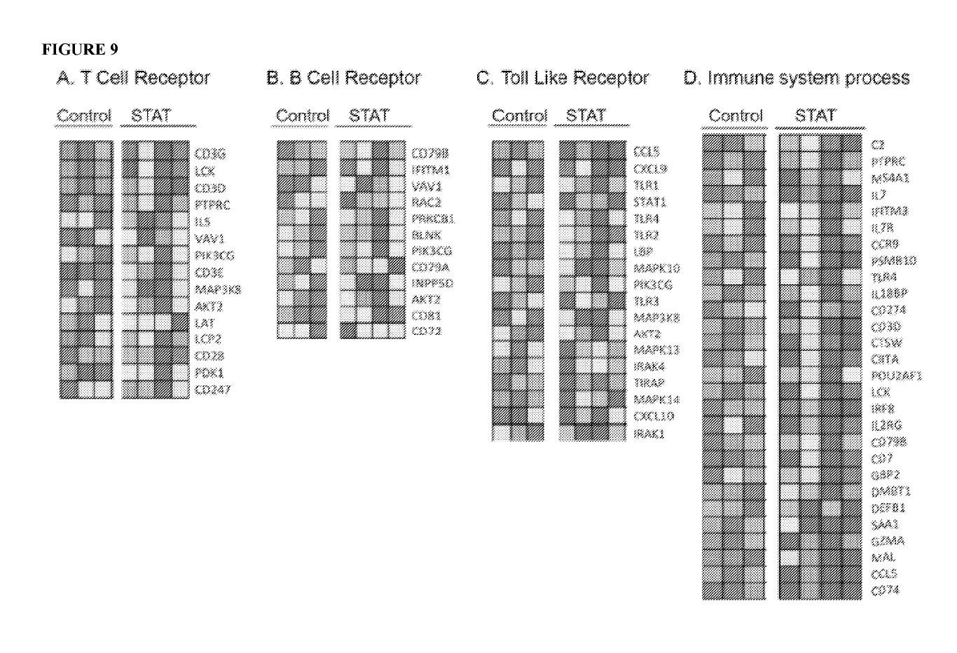

FIG. 9 shows genes from the ileal tissues from the mice in FIGS. 1-5 (Control, n=3; STAT n=4) that are down-regulated in STAT, and are involved in immunity, including T cell receptors (FIG. 9A), B cell receptors (FIG. 9B), Toll like receptors (FIG. 9C), and immune system processes (FIG. 9D). These data provide examples of global down-regulatory effects, involving both adaptive and innate arms of immunity. Note strong down-regulation of SAA1 by STAT.

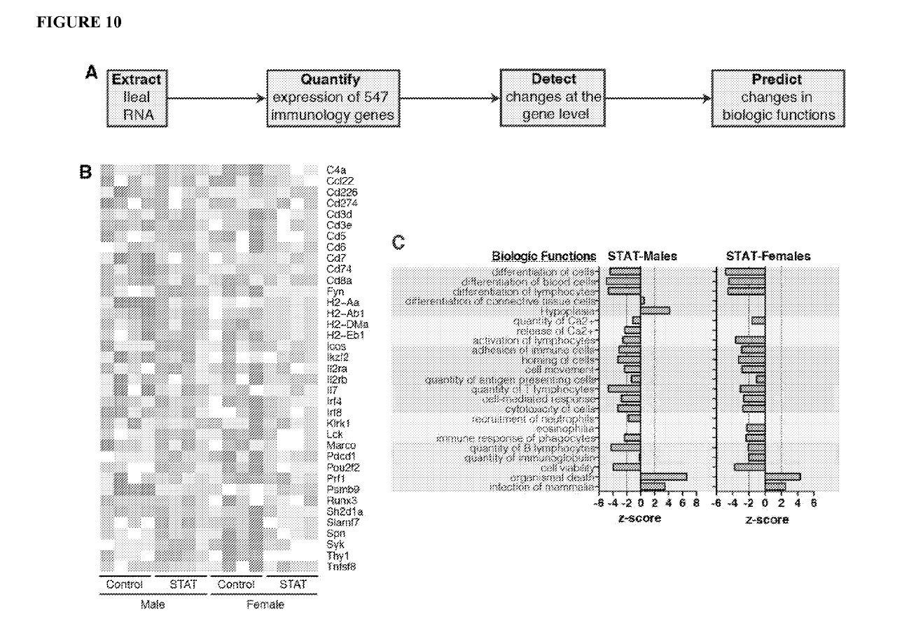

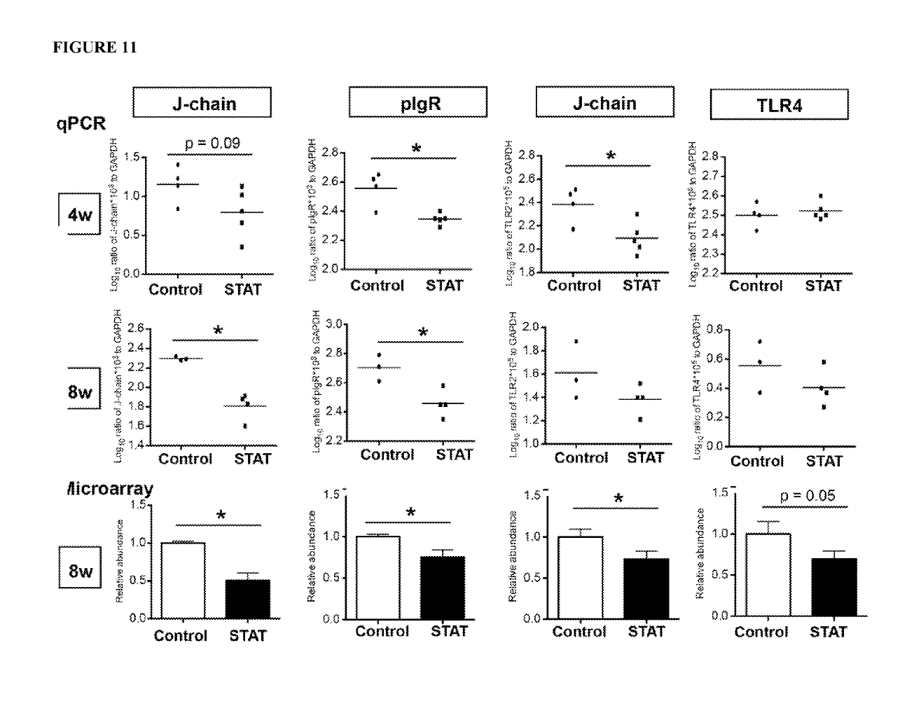

FIG. 10 shows the results of transcriptional profiling analysis of intestinal tissue by Nanostring analysis. These results reveal that STAT was associated with a general decreased expression of genes involved in intestinal immune responses. Specifically, STAT decreased 111 and 74 genes and increased only 1 and 7 genes in male and female mice, respectively, with numerous consistencies across gender (FIG. 10A-C and Table 2). This indicates that the STAT treatment resulted in widespread down-regulation of immune related genes in both males and females, and conservation across gender increases the confidence of these findings. Ingenuity Pathway Analysis revealed that STAT decreased expression of genes related to several biologic functions, such as differentiation, activation, recruitment, and adhesion of immune cells, and functions specifically related to antigen-presenting cells, T-cells, B-cells, and phagocytic cells (FIG. 10A-C). These results demonstrate that STAT decreases markers of intestinal immunity.

TABLE 2
Ileal immune gene expression

| | Ileal Nanostring | | |
|---|---|---|---|
| Significant^{a} Changes | Up | Down | Total |
| STAT-Male | 1 | 111 | 112 |
| STAT-Female | 7 | 74 | 81 |
| Recipient-Female | 5 | 16 | 21 |
| Shared: STAT-M + STAT-F | 0 | 37 | 37 |
| Shared: STAT-F + Recip-F | 0 | 1 | 1 |
| Shared: STAT-M + Recip-F | 0 | 3 | 3 |

F = Female, M = Male,
STAT = effect of sub-therapeutic antibiotic treatment (vs. control) on ileal gene expression in 8-week old mice, n = 4 per group.
Gene expression in STAT and control ileal specimens measured by the Nanostring
Immunology Recip = recipient; effect of transferring control or LDP microbiota to germ-free recipients
^{a}Significant: t-test <0.05 for nanostring.

Ileal expression of the IgA J-chain, polymeric Ig receptor (pIgR), both related to polymeric IgA function, and TLR2 and TLR4 was quantitated by qPCR and normalized by GAPDH expression in 4- and 8-week STAT [4-week; Control n=3, STAT n=4, 8-week; Control n=3, STAT n=4]. Both qPCR and microarray data showed significantly down-regulated B cell markers with a downward trend in TLRs in STAT mice at both time-points compared to controls (FIG. 11). This example shows that the effects of the low dose antibiotic exposures are not limited to T cells, but also affect B cells and innate immunity.

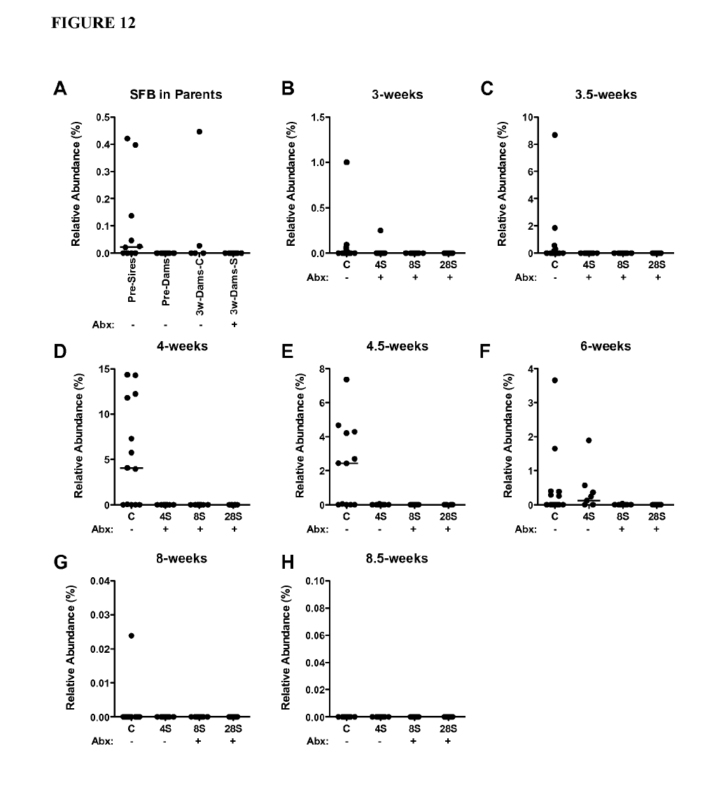

Because prior studies indicated a role of segmented filamentous bacteria (SFB) in immune phenotypes (Ivanov et al., 2009, Cell, 139:485-98), the present inventors characterized the SFB populations in the intestine by high throughput (MiSeq Illumina) sequencing of fecal pellets at an average read depth of 5,800 quality-filtered reads/specimen. The Green Genes May 2013 version includes SFB in their annotation. C57BL6J mice were bred, and 1 week prior to birth, 12 of the 17 mothers were exposed to sub-therapeutic antibiotic treatment (S, STAT), while 5 did not receive antibiotics (C, Control). Control offspring did not receive antibiotics, STAT offspring received STAT for 4, 8, or 28 weeks. All offspring were switched to a high fat diet at 6 weeks of age. FIG. 12 shows SFB relative abundance in (A) parents, and (B-H) offspring at 3 to 8.5 weeks of age. Several sires were SFB-positive, and 3 weeks after birth, one of the dams had low level positivity. From 3 to 4.5 weeks, some of the control pups were positive and then numbers declined, so that by 8.5 weeks there were no detectable SFB in any of the pups that were examined for immune characteristics. Some of the 4-STAT mice had low levels of SFB detected at 6 weeks (2 weeks after ending antibiotics), but levels were not detectable in the other STAT mice, or in all but one Control mouse at 8 weeks. These data provide evidence that the presence of SFB cannot explain the differences in immune phenotypes between STAT and Control that were observed.

### Example 2

### TRANSTAT—Transmission of Altered Immune Phenotype Through Microbiota Transfer

Example 1 demonstrates strong associations between administering antibiotics and changes in intestinal immune functions. But to develop practical approaches to issues of immunity, it is important to determine whether antibiotics are working directly on the tissues or whether the effect of the antibiotics is mediated through their effects on microbiome composition. To determine this, microbiota from the STAT-exposed mice and the Control mice were harvested and transferred into germ-free mice. Transfer of microbiota to germ-free animals allows examining the characteristics of the microbiota, independent of any on-going host or drug effects. After the germ-free mice became “conventionalized” (i.e., colonized by a microbiota), the effects of the alternate sources of their microbiota on their immune characteristics were determined.

C57BL/6J (Jackson Labs, Bar Harbor, Me.) mice either received no antibiotics (control) or continuous STAT penicillin. Mice were weaned at 4 weeks onto normal chow (13.2% fat, 5053 PicoLab Rodent Diet 20, LabDiet, Brentwood, Mo.), then changed to a high fat diet (45% kcal from fat, D12451, Research Diets, New Brunswick, N.J.) at 6 weeks of life. At 18 weeks of age, from each group (Control, n=9; STAT, n=8), the three animals with weight at or closest to the median were selected as cecal content donors. Donor mice were humanely euthanized, and the proximal ⅓ of the cecum was aseptically removed and after a series of processing steps, either the pooled Control or STAT microbiota suspensions were placed in the stomachs of the germ-free recipients (Control, n=7 recipients; STAT, n=8) by oral gavage. Recipients were chosen randomly, and the inoculum alternated between control and STAT recipients. Mice awoke from anesthesia within minutes and no mouse exhibited ill effects from the microbiota transfer. The microbiota-recipient mice were housed in autoclaved cages, under specific pathogen-free conditions, and fed an irradiated high fat diet (45% kcal from fat, D12451, Research Diets, New Brunswick, N.J.), and followed for the next 35 days until sacrifice. Fecal pellets were collected serially from the time of transfer.

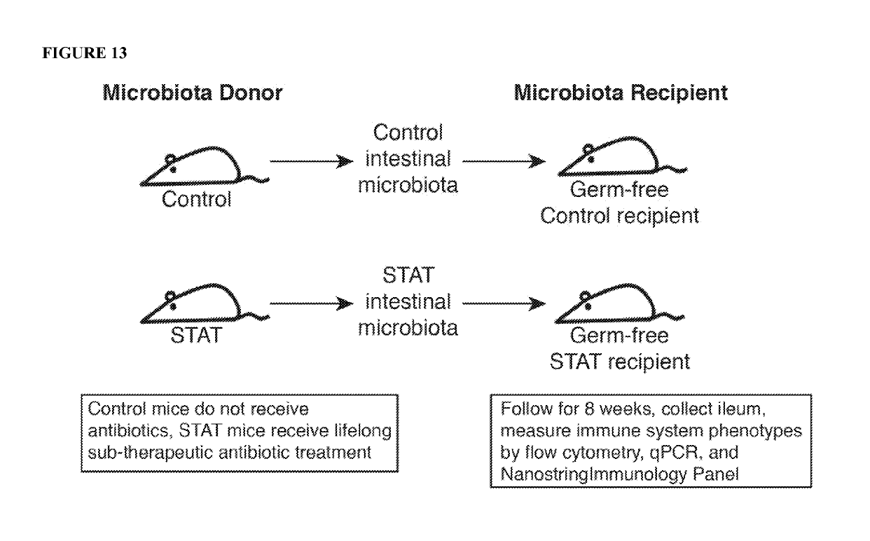

C57BL6J mice either did not receive antibiotics (Control) or received sub-therapeutic antibiotic treatment with penicillin (STAT) from birth until 18 weeks of age. Mice were fed normal chow, then switched to high fat diet at 6 weeks of age. At 18 weeks, cecal contents were collected from 3 control and 3 STAT mice, based on their median weight, pooled, and transferred to 3-4 week old germ-free Swiss Webster mice by oral gavage. Every attempt was made to maintain viability of the microbiota by protecting the microbiota from oxygen and minimizing time between microbiota collection and transfer. Microbiota recipient mice were monitored for 5 weeks, then ileal samples were collected to assess immunity by flow cytometry and qPCR. Ileal gene expression was also measured by microarray and Nanostring Immunology Panel (FIG. 13).

C57BL6J mice received sub-therapeutic antibiotic treatment (STAT; n=3), Control mice did not receive antibiotics (n=3). At 18 weeks, microbiota was transferred to germ-free Swiss-Webster mice (Control-microbiota recipients, n=7; STAT-microbiota recipients, n=8).

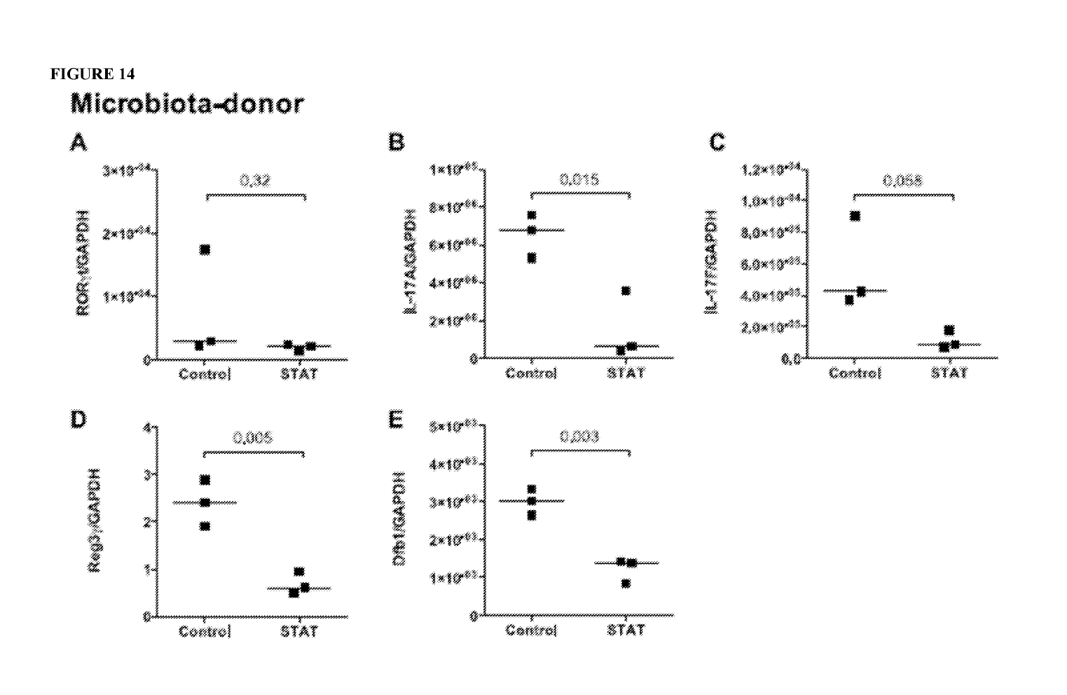

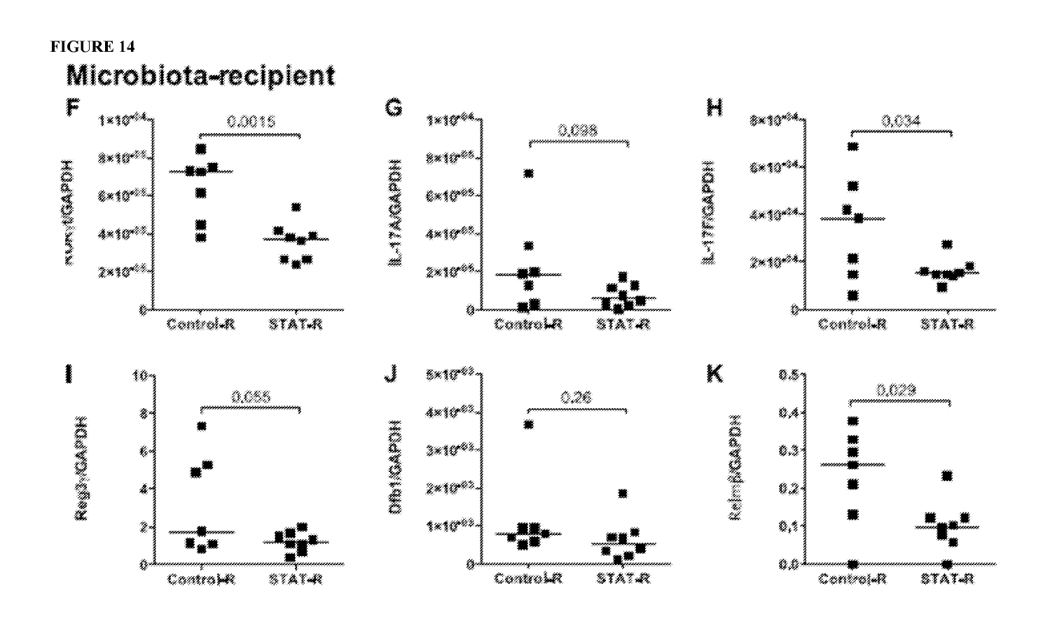

FIG. 14 shows ileal expression of Th17 cell transcription factor (RORγt), Th17 cytokines (IL17-A and IL-17F), and antimicrobial peptides in 18-week old control and STAT microbiota donor mice (Panel A-E) and 8-week old control- and STAT-microbiota recipient mice (Panel F-K). Expression levels were measured by qPCR and normalized by GAPDH expression.

In the STAT-donor mice, there was significantly down-regulated expression of IL-17A, Reg3γ, and defensin β1, and a downward trend of RORγt and IL-17F expression in the ileum. In the recipients of the STAT microbiota, compared to the recipients of the Control microbiota, there were significant decreases in the Th17 markers RORGT and IL-17F, and the antibacterial peptide RelmB, and consistent trends of decrease in IL-17A and Reg3G. This example provides evidence that the antibiotic-altered microbiota composition was sufficient to transfer the altered immune phenotypes to the recipient germ-free mice.

Gene Expression Profiling Via Nanostring Immunology Panel.

Microbiota from 3 control or STAT donors was transferred to 3-week old germ-free Swiss-Webster mice, and ileal tissue was collected at 8 weeks of age (FIG. 13). RNA was extracted from ileal tissue at 8 weeks of age using the miRNeasy mini kit (Qiagen, Redwood City, Calif., USA) and the expression levels of 547 genes related to immunology were measured by nCounter GX Mouse Immunology Kit (Nanostring Technologies, Seattle, Wash., USA. Significant differences between treatment and control were determined by t-test. For functional characterization, the significant changes were analyzed by Ingenuity Pathway Analysis (Qiagen, Redwood City, Calif.).

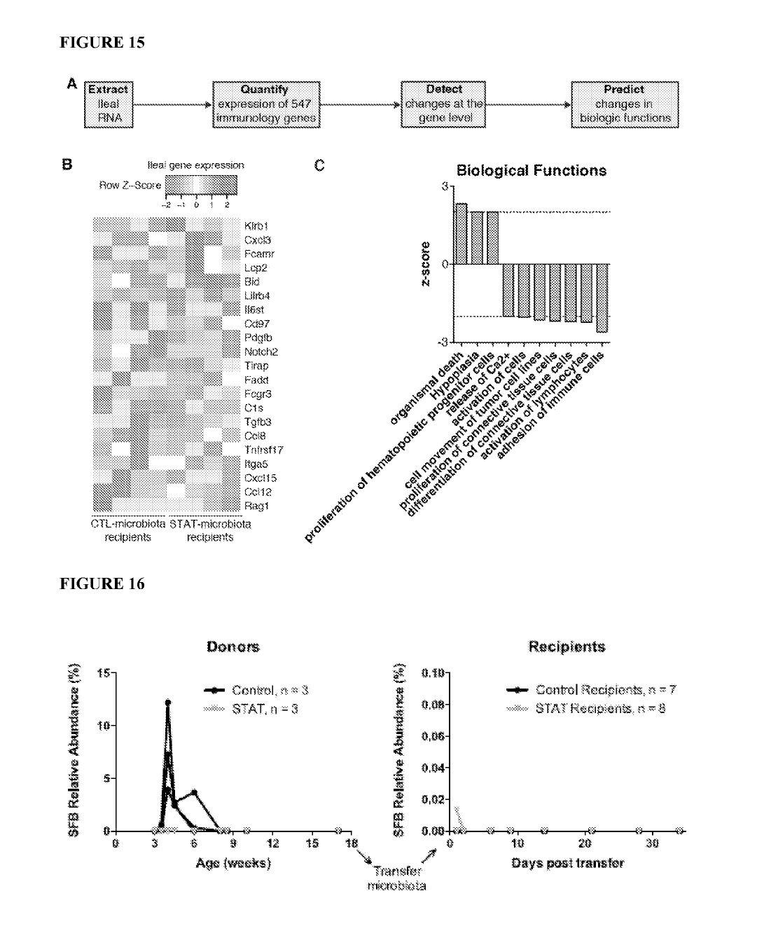

To test whether altered microbiota could induce changes in intestinal immunity, cecal microbiota was collected from 3 control and 3 STAT mice at 18 weeks of age and transferred into germ-free Swiss Webster mice (FIG. 13). Changes in ileal gene expression were measured by the Nanostring Immunology Panel, and STAT microbiota recipients had more genes decreased (21) than increased (5), similar to their microbiota donors (FIG. 15 and Table 2 of Example 1). These changes were related to predicted biological functions similarly altered in the 8-week old male and female mice directly exposed to STAT (FIG. 15), including decreased differentiation, activation, and adhesion of immune cells. These studies provide evidence that altered microbiota can mediate changes in intestinal immune gene expression and are consistent with the global reduction in intestinal immune responses observed in the STAT-exposed microbiota donors.

C57BL6J mice received sub-therapeutic antibiotic treatment (STAT), or did not receive antibiotics. As above, mice were weaned onto normal chow at 4 weeks and switched to HFD at 6 weeks of age. Cecal microbiota was collected at 18 weeks of age and transferred to 3 to 4-week old germ-free Swiss-Webster mice, which were then housed in specific pathogen-free (SPF) conditions and fed HFD. Longitudinal fecal samples were collected and microbial communities were profiled by 16S rRNA sequencing using standard universal primers (Caporaso et al., ISMEJ 2012, 8:1621-4) at an average depth of approximately 5,800 sequences per sample.

### Library Preparation:

Forward primer:
(SEQ ID NO: 39)
AATGATACGGCGACCACCGAGATCTACACTATGGTAATTGTGTGCCAGC
MGCCGCGGTAA
Reverse Primer:
(SEQ ID NO: 40)
CAAGCAGAAGACGGCATACGAGATNNNNNNNNNNNNAGTCAGTCAGCCG
GACTACHVGGGTWTCTAAT

### Sequencing Primers:

Forward Primer:
(SEQ ID NO: 41)
TATGGTAATTGTGTGCCAGCMGCCGCGGTAA
Reverse Primer:
(SEQ ID NO: 42)
AGTCAGTCAGCCGGACTACHVGGGTWTCTAAT
Index:
(SEQ ID NO: 43)
ATTAGAWACCCBDGTAGTCCGGCTGACTGACT

Taxonomy was assigned based on the Green Genes May 2013 database, and the percentage of sequences classified as Candidatus Arthromitus, the candidate genus name for segmented filamentous bacteria (SFB), was calculated. FIG. 16 shows SFB relative abundance in the microbiota-donors (A), and the microbiota-recipient (B) mice, and each mouse is plotted. SFB was present in all 3 control donor mice at levels >1%. SFB was undetectable in STAT donors at all time points, and in the Control donors following the switch to a HFD (8 weeks and beyond). SFB was nearly undetectable in microbiota recipients. Only one STAT-recipient mouse had SFB detected, and it was only one sequence that was classified as SFB out of 7,657 total sequences. SFB was not detected in any of the 7 Control or the other 7 STAT microbiota-recipients. This experiment clearly shows that the differences in immune phenotypes observed following the transfer to the germ-free mice, cannot be explained by transfer of SFB. Other microbiota taxa are thus important in the transferred phenotype.

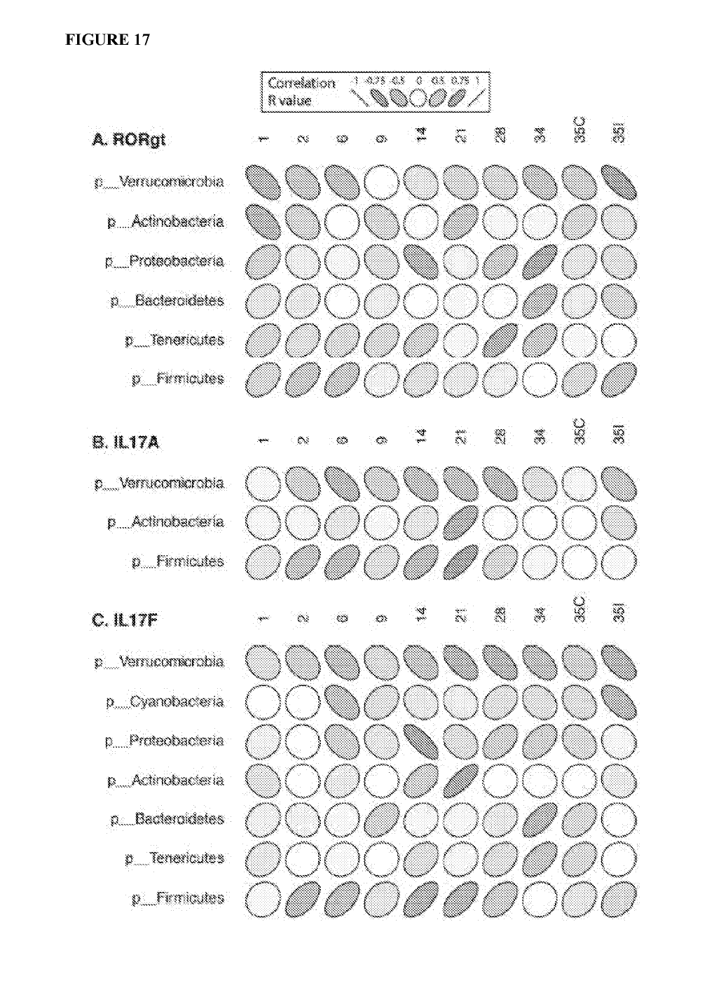

Germ-free Swiss Webster mice were colonized with microbiota from Control mice or STAT mice, as above. The intestinal microbiota was surveyed over time by high throughput sequencing at an average depth of approximately 5,800 sequences per sample. The Spearman correlation between (A) RORγT, (B) IL-17A, and (C) IL-17F ileal expression at 35 days-post transfer with the relative abundance of the predominant phylum over time was calculated. Phyla with at least one significant correlation (p<0.05) are shown in FIG. 17. This experiment shows that there are microbes from different phyla that are either positively or negatively correlated with the observed day 35-post-transfer phenotypes. These taxa are indicators of the microbe types that could be transferred to alter immune response of a host in a particular direction.

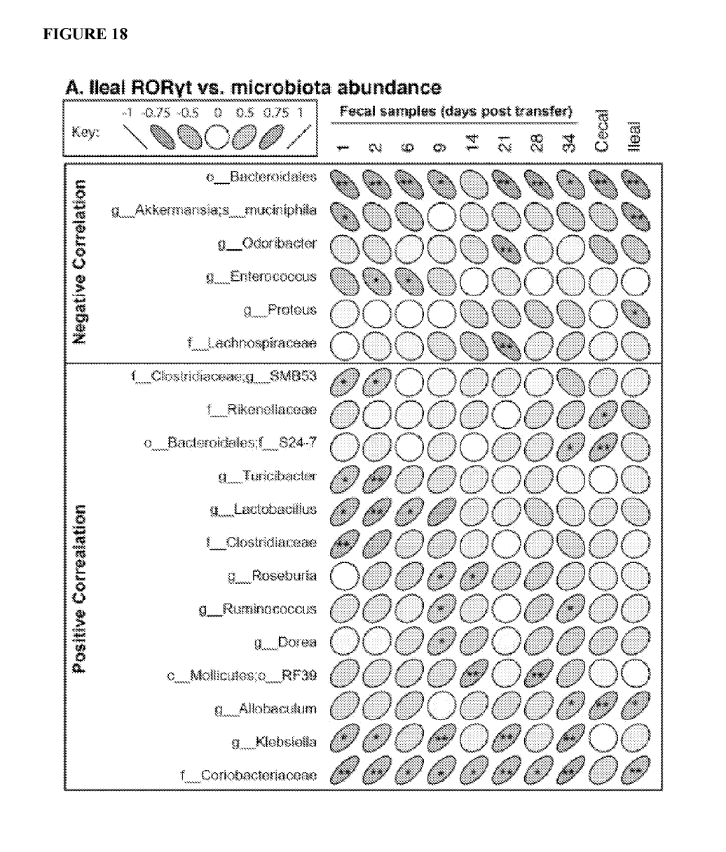

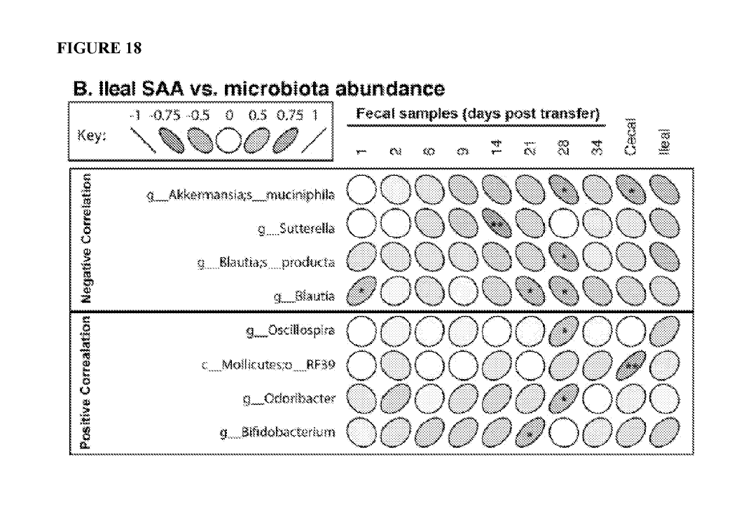

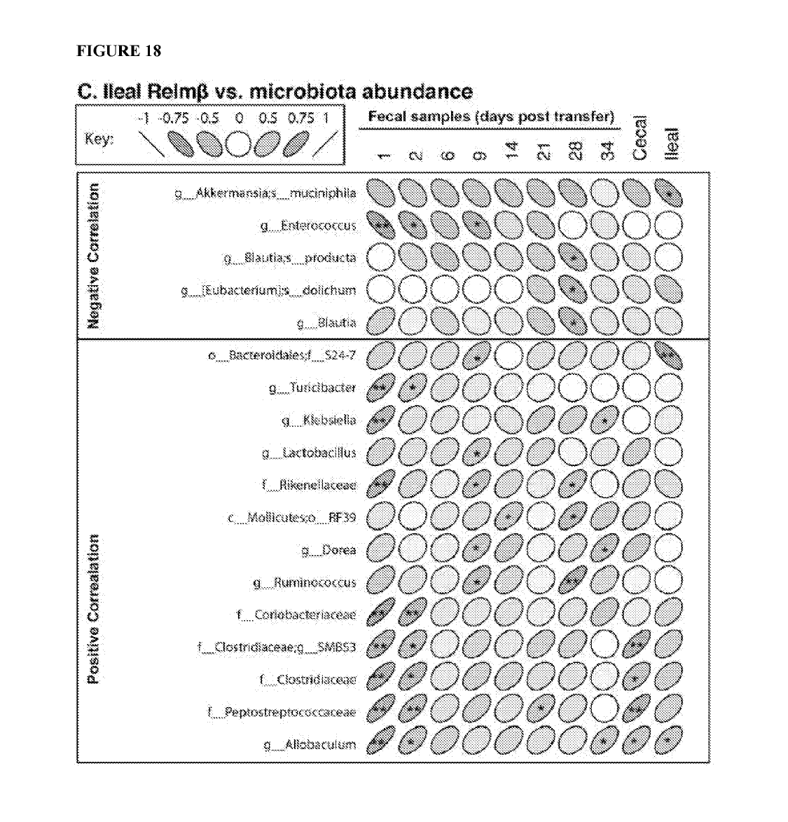

Germ-free Swiss Webster mice were colonized with microbiota from control mice or STAT mice. The intestinal microbiota was surveyed over time (1-34 days post transfer fecal specimens, 35 days post transfer cecal and ileal specimens) by high throughput sequencing at an average depth of approximately 5,800 sequences per sample. Taxonomic assignment was made through the QIIME pipeline using the May 20, 2013 Green Genes database of 16S microbial sequences. The Spearman correlation was calculated between (A) ileal RORγT, (B) SAA1/2, and (C) Relmβ expression at 35 days-post transfer with relative abundance of the predominant species (>1% in any sample). Microbiota with at least one significant correlation (p<0.05), and consistent correlation direction are shown in FIG. 18. In FIG. 18, an ellipse with a forward slant represents a positive Spearman correlation, and a backwards slant represents a negative Spearman correlation, and the narrowness of the ellipse indicates the strength of the correlation (higher rho value). Significant correlations are indicated as * p<0.05, ** p<0.01. Microbiota names are reported at the lowest possible identifiable level, indicated by the letter preceding the underscore: o=order, f=family, g=genus, s=species. This experiment defines the significant taxa down to the genus level in most cases, and including the species level, and represents candidate microbiota for manipulating immune responses.

### Example 3

### The Effect of Pulsed Antibiotic Treatment (PAT) on Immunity

The prior examples relate to manipulation of the microbiome by continuous exposure to low doses of antibiotics. In this example, the present inventors addressed giving discrete pulses of antibiotics for a few days at a time in relation to affecting immune phenotypes.

C57BL/6 mice were fed a standard diet after weaning, and exposed to 3 pulses of therapeutic dose antibiotics (Amoxicillin or Tylosin), or no antibiotics, via their drinking water, all before 6 weeks of age. Mice were sacrificed at 6 weeks of age.

Tissue Collection.

The distal ileum (1 cm) without Peyer's Patches was collected from each mouse for RNA extraction and kept in RNAlater (Ambion) overnight at 4° C. Samples were stored at −80° C. until RNA extraction was performed.

LPL Lymphocyte Preparation.

Small and large intestines of mice were dissected, and all mesenteric fat and Peyer's patches were removed. Intestines were fileted for removal of stool, cut into 2 cm pieces, and washed with PBS. Tissues were transferred to tubes with 10 ml 1 mM DTT/PBS, shaken at RT for 10 minutes, and transferred to tubes with 30 mM EDTA and 10 mM HEPES in PBS. Intestines were then shaken at 37° C. for 20 minutes and washed in complete RPMI. The small and large intestines were digested at 37 C for 1.5 hours in 100 units/ml collagenase Type VIII (Sigma) and 150 μg/ml DNase (Sigma) in complete RPMI. Cells were filtered through a 50 micron filter, washed with 5 ml PBS, and pelleted. Cells were then resuspended in 40% Percoll (GE Healthcare), under-layed with 80% Percoll, and centrifuged at 2,200 rpm for 20 min at RT. Lamina propria lymphocytes were collected at the interface and used for flow cytometric analyses.

FACS Staining.

Cells were stimulated with phorbol 12-myristate 13-acetate (PMA) and ionomycin for 4 hours at 37 C with the addition of GolgiPlug (BD). Following this in vitro stimulation, cells were stained with a live/dead Aqua marker, anti-CD3, anti-CD4, anti-CD8, and anti-NKp46 and fixed in 4% paraformaldehyde in PBS for cytokine staining and in eBioscience Fix/Perm for nuclear staining Cells used in cytokine staining were permeabilized in Perm/Wash buffer (BD) and stained with anti-IFNγ, anti-IL-17A, anti-IL-22, anti-IL-4, and anti-TNFα. Cells used in nuclear staining were permeabilized in Perm/Wash (eBioscience) and stained for FoxP3, Ki67, GATA3, RORγT, and Tbet. Cells were acquired on an LSRII (BD) and analyzed with FlowJo (Tree Star, Inc.) software.

Histology.

Ileal tissues from 4-week-old control and STAT mice were formalin fixed and paraffin embedded, cut, and stained with hemotoxylin and eosin (H&E). 4 slides were scored for ileal atrophy per mouse and the average score per mouse was averaged.

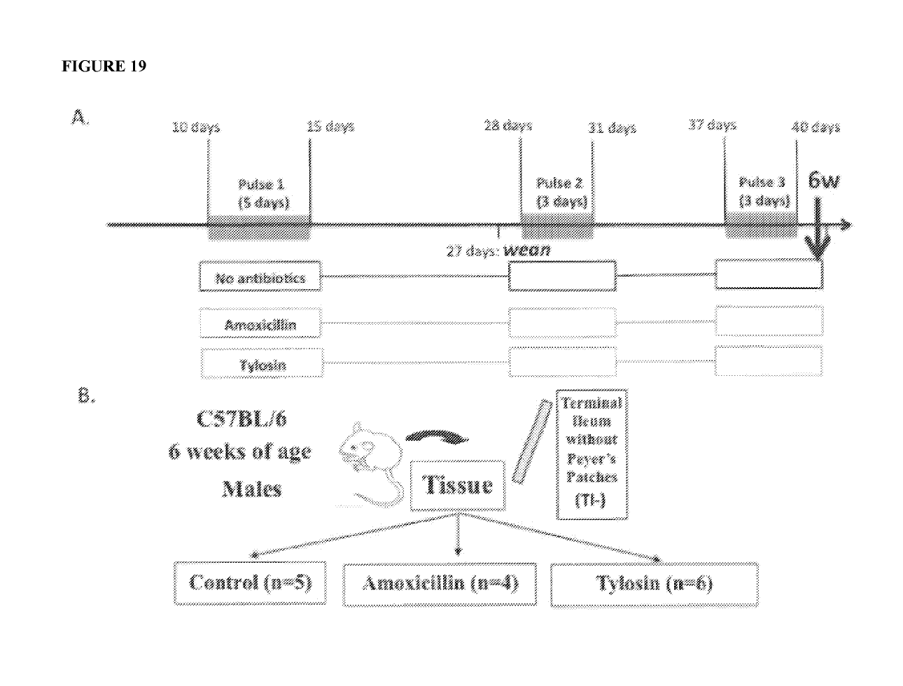

FIG. 19 shows a schematic of pulsed antibiotic treatment (PAT) schedule (Panel A) and study groups for gene expression analysis (Panel B). Panel A: Mice were exposed to 3 pulses of antibiotics (Amoxicillin or Tylosin), or no antibiotics, via breast milk or drinking water at the indicated time points. Panel B: Male mice, including 5 Control, 4 Amoxicillin, and 6 Tylosin, were fed a normal diet. They were sacrificed at 6 weeks of age and the terminal[1 cm] ileum without Peyer's Patches [TI-] was collected.

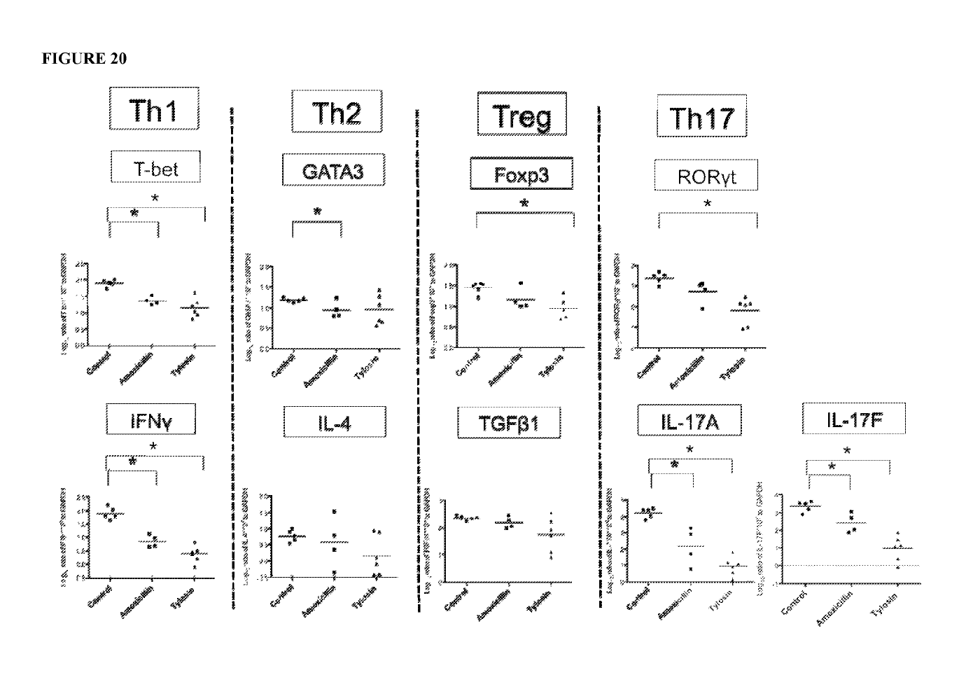

Ileal expression of T-bet and IFN-γ [Th1], GATA3 and IL-4 [Th2], Foxp3, and TGFβ1 [Treg], and RORγt, IL-17A, and IL-17F [Th17] (Control n=5, Amoxicillin n=4, Tylosin n=6) was quantitated with qPCR and normalized by GAPDH expression. Expression of T-bet and IFNγ [Th1] (p<0.05) and IL-17A and IL-17F [Th17] (p<0.05) was significantly lower in both Amoxicillin and Tylosin compared to control (FIG. 20). Foxp3 and RORγt were lower in Tylosin than control. This example extends the earlier observation that continuous antibiotics affect ileal immune cell populations and effectors by showing that pulses are sufficient to produce similar effects.

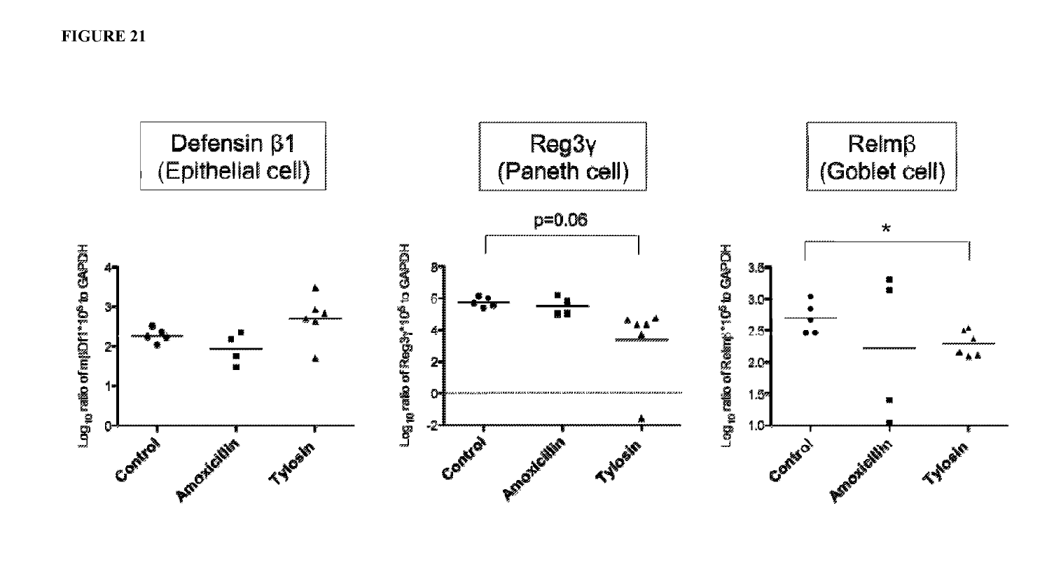

Ileal expression of defensin β1, Reg3γ, and Relmβ (Control n=5, Amoxicillin n=4, Tylosin n=6) was quantitated by qPCR and normalized by GAPDH expression. Relmβ expression was significantly lower in Tylosin compared to control mice (p<0.05) (FIG. 21). This experiment shows that pulse antibiotic exposure is sufficient to alter antibacterial peptide gene expression.

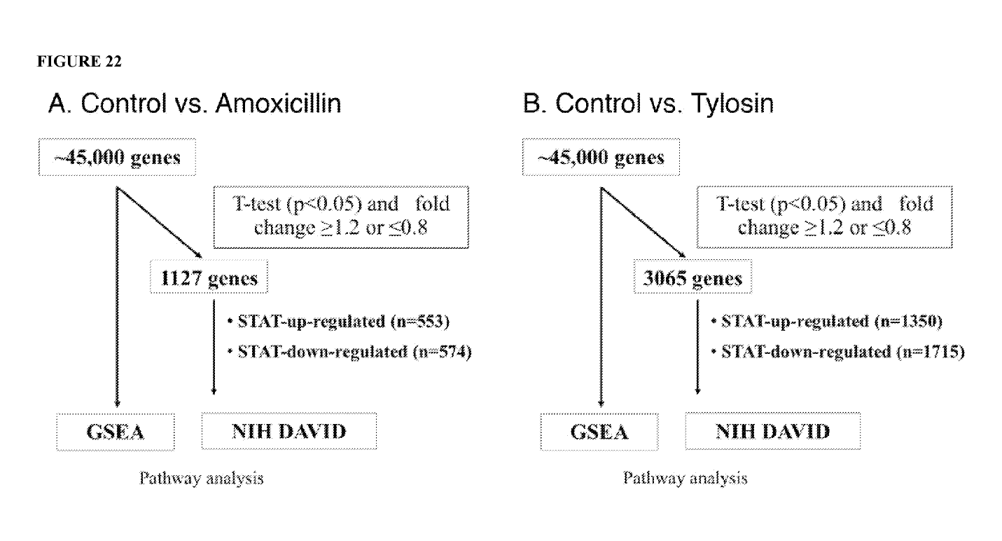

1127 and 3065 reproducibly regulated genes were identified from ˜45,000 genes of the mouse genome by T-test (p<0.05) and fold change (≧1.2 or ≦0.8) in 6-week PAT (Panel A; Control vs Amoxicillin and Panel B; Control vs Tylosin), respectively (FIG. 22). DAVID analysis was performed using the regulated genes, and Gene Set Enrichment analysis (GSEA) was performed using whole gene data. This experiment shows that pulsed antibiotic treatments can have large-scale effects on gene expression in the terminal ileum.

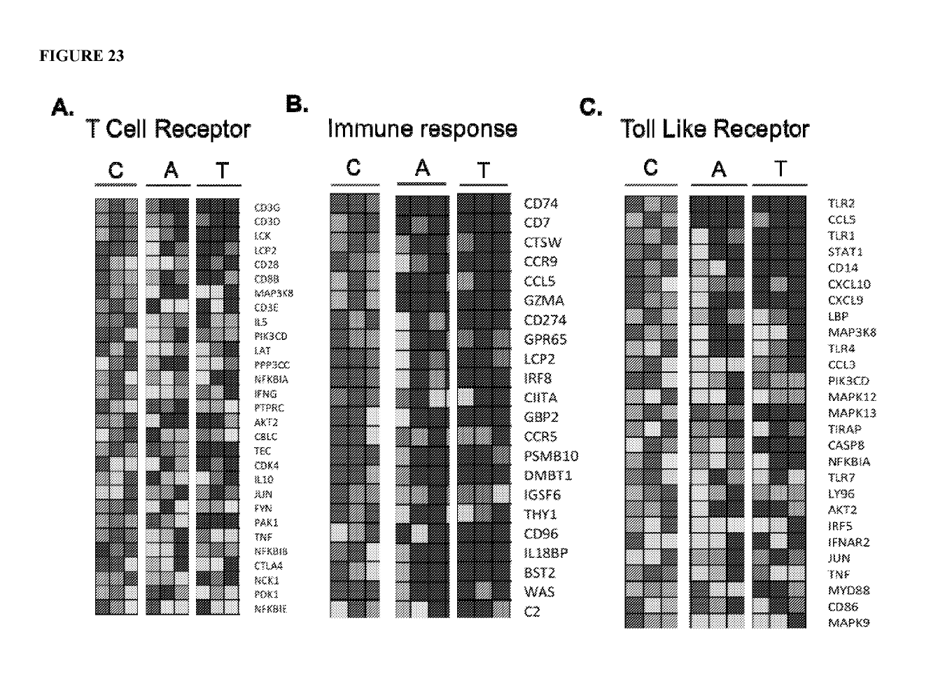

(C, n=3), amoxicillin (A, n=3), and tylosin treated mice (T, n=3). FIG. 23 shows pathways down-regulated in both 6-week PAT mouse groups, and involved in immunity, including (A) T cell receptor pathway, (B) immune responses, and (C) Toll like receptor. This experiment shows that changes in gene expression induced by PAT exposures are highly similar to those due to STAT exposures.

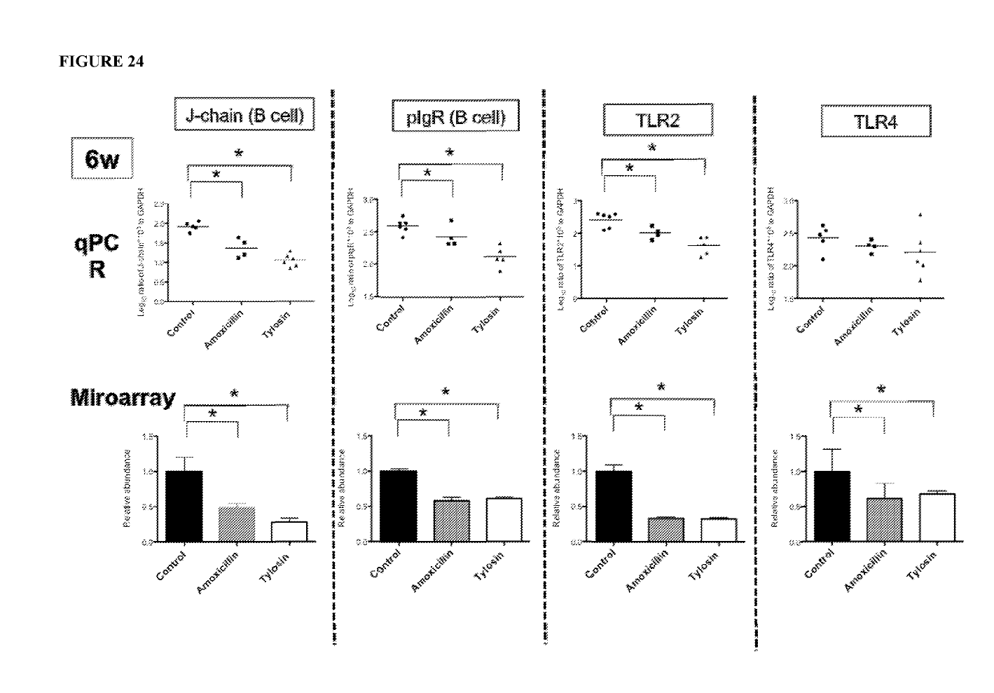

Ileal expression of J-chain, polymeric Ig receptor (pIgR), both related to polymeric IgA function, and TLR2 and TLR4 was quantitated by qPCR and normalized by GAPDH expression. Control n=5, Amoxicillin n=4, Tylosin n=6. Both qPCR and microarray data showed significantly down-regulated B cell markers with a downward trend in TLRs in PAT mice compared to controls (FIG. 24). This experiment shows that changes in PAT are similar to those observed in STAT manipulations.

### Example 4

### Early Life (EL)-PAT

In this example, the effects of starting the pulse very early in life (at day 5) were investigated to determine whether three pulses are needed for the effect, or whether a single early life pulse is sufficient for the effect.

Animals.

7-week old male and female C57BL/6 mice (Jackson Labs, Bar Harbor, Me.) were bred in the NYUMC vivarium to obtain sufficient litters for the study. Mice were maintained on a 12-hour light/dark cycle and fed standard laboratory chow (Purina Mills International Diet no. 5001) and water ad libitum.

Antibiotics.

For the experiments, dams and their litters were divided into control and antibiotic groups. In the EL-PAT study dams in the antibiotic groups received Tylosin tartrate (Sigma Aldrich, St. Louis Mo.) dissolved in distilled deionized water (neutral pH) at a concentration of 0.33 mg/ml, and dams in the control group received non-acidified water. Mice in the PAT3 group received two additional doses of antibiotics at day 27 and 36 of life for 3 days. In some experiments, C57BL/6 mice were exposed to one, PAT1, (n=17, M=9, F=8) or three, PAT3, (n=19, M=10, F=9) doses of the macrolide Tylosin in non-acidified water or non-acidified water alone (control, n=12, M=9, F=3), early-in-life, to mirror pediatric antibiotic exposures. For the first antibiotic pulse 0.333 mg of Tylosin tartrate (Sigma) was dissolved in 250 ml of non-acidified water and given to PAT1 and PAT3 lactating dams at pup day 5 of life for 5 days. For the second and third pulse, pups received the antibiotic directly at day 27 and day 36 of life respectively, for three days. Mice were sacrificed at 7 weeks of age and fecal, ileal and cecal contents were collected. Throughout the experiment mouse weight and fecal contents were collected.

Isolation of Small Intestine Lamina Propria Lymphocytes.

After euthanasia of the mice by CO₂ asphyxiation, small intestines were excised and placed in cold calcium and magnesium-free HBSS supplemented with HEPES-Bicarbonate and 5% FBS (CMF). Peyer's patches were removed and the intestine was opened longitudinally along its entire length. Intestinal contents were removed by washing thoroughly with cold CMF and treated with 1 mM DTT to remove contaminating intra-epithelial lymphocytes. Subsequently, the intestinal tissue was treated twice with 1.3 mM EDTA to remove epithelial cells. To isolate lamina propria lymphocytes, intestinal tissue was cut laterally into ˜0.5 cm pieces and digested in collagenase IV digestion mix (0.5 mg/ml of collagenase IV, 200 ug/ml of DNase 1 suspended in RPMI supplemented with 10% FBS and HEPES for 1 hour. For each digestion procedure, samples were incubated at 37° C. with vigorous shaking After each treatment, cell isolates were filtered through 100 μm diameter nylon mesh (BD Bioscience).

Enrichment of Leukocyte Population Via Density Gradient Centrifugation.

For further purification of leukocytes, intestinal cell suspensions were subjected to a 44% /67% discontinuous Percoll (GE Healthcare Life Sciences) gradient. Cells were spun at 2,200 rpm for 20 minutes and collected at the 44% /67% Percoll interface. Viable cells were carefully removed from the interface, washed, and suspended in RPMI complete media.

Primary Culture Preparation and Lymphocyte Staining.

To induce cytokine production, intestinal leukocytes were cultured for 10 hours in RPMI complete media containing brefeldin A (eBioscience) and 1 μg/μl of bound anti-CD3/and soluble anti-CD28 (BD Bioscience). After stimulation, cells were washed with PBS and incubated with blue Live/DEAD fixable dye (Invitrogen) for 10 minutes to identify live cells. Intestinal leukocytes were phenotyped using the following antibodies: CD45-Percp-Cy5.5, APC-Rorgt, PE-IL-17A, CD4-V500, FITC-IFNγ (BD Biosciences), CD8-BV650 (Biolegend), and Foxp3-PE-Cy7 (eBioscience). For surface and intracellular staining, cells were first incubated with surface antibodies for 30 minutes at 4° C., fixed with Fix/Perm (eBioscience) and subsequently incubated with intracellular antibodies in perm/wash buffer for 30 minutes at 4° C. (eBioscience).

Analysis of Lymphocyte Subsets.

All cells were acquired on a LSRII (BD Bioscience) and analyzed using FlowJo software (version 9.3.2; Tree Star Inc.).

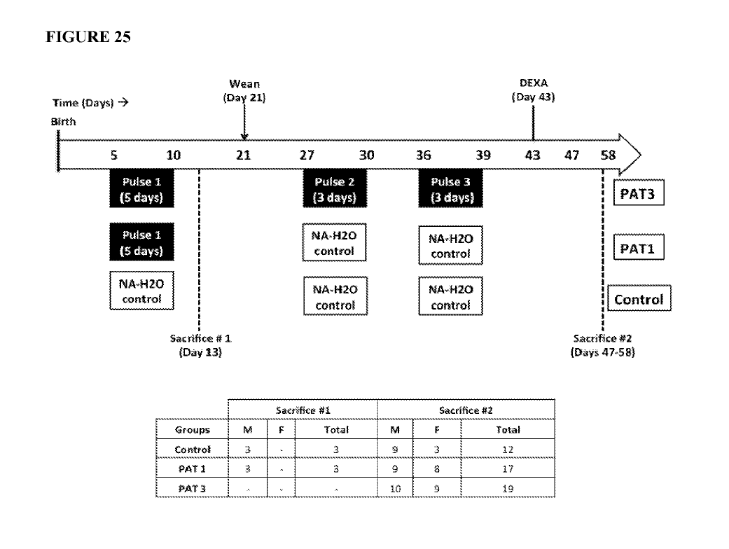

FIG. 25 shows a schematic of early-life pulsed antibiotic treatment (EL-PAT) study. C57BL/6 mice were treated with either one (PAT1) or three (PAT3) therapeutic doses (50 mg/kg/day) of tylosin tartrate. Antibiotics were administered in non-acidified water at day 5 of life for 5 days, and for the PAT3 mice, at day 27 and day 36 of life for 3 days. Control mice were given non-acidified water during each treatment. Three male control and antibiotic-treated mice were sacrificed at day 13 of life, and at week 7 (day 50+/−6) of life, all groups were sacrificed.

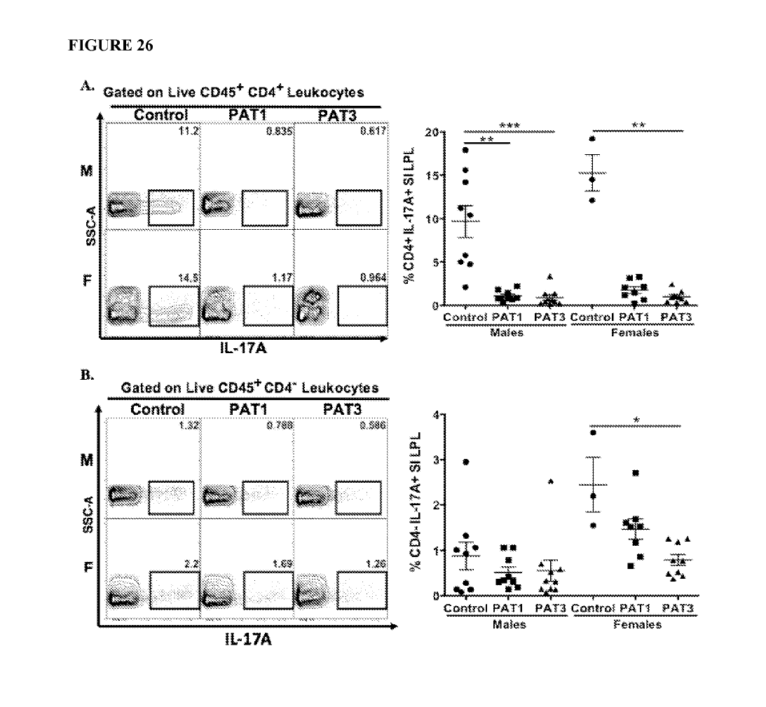

Small intestine lamina propria lymphocytes were isolated from 7-week old control and PAT mice. Mice were sacrificed at 7 weeks of age and fecal, ileal and cecal contents were collected. Throughout the experiment, mouse weight and fecal contents were collected. Intestinal CD4⁺ IL-17A⁺ and CD4− IL-17A populations were phenotyped based on surface and intracellular staining Cells were gated on CD45+ and either CD4+ or CD4-populations. FIG. 26A shows that IL-17A expression was significantly decreased with one or three therapeutic doses of antibiotics in males. ***p<0.001, **p<0.01, Three antibiotic doses significantly decreased intestinal CD4⁺ IL-17A⁺ populations in females. (A)**p<0.01. Additionally, FIG. 26B shows that three therapeutic doses of antibiotics decreased CD4⁻ IL-17A⁺ populations in females. *p<0.05. This example shows that even a single pulse of antibiotics early in life was sufficient for the manipulation, and also that the effect of a pulse that ended at day 10, persisted for another 6 weeks of life; to the time of sexual maturity of the mice. Thus, the effect of the single pulse was long-lasting. In this example, using three pulses rather than one had no added benefit.

EL-PAT DNA Extraction and Amplicon Library Preparation.

Fecal, ileal and cecal DNA were extracted using Mobio 96-well extraction kit (MO BIO Laboratories, Inc., CA). For amplicon library generation, the V4 region of 16s rRNA gene was amplified with gene specific primers (SEQ ID NOS: 39-43). Amplicons were prepared in triplicate, pooled and quantified. The 254 bp V4 region was sequenced using the Illumina MiSeq 2×150 bp platform (Illumina Inc., CA). Forward and reverse reads were joined using the ea-utils module fastq-join. Demultiplexing and operational taxonomic unit (OTU) picking were performed using the bioinformatic tool Qiime, while pair-end reads were demultiplexed with the script split_libraries_fastq.py. Reads were quality filtered at a phred score of 20 using -q flag, and the flag --rev_comp_mapping allowed reading of the reverse complement barcode before demultiplexing. Open referenced OTU picking was performed using pick_open_reference_otus.py script to assign a taxonomy for each OTU representative sequence and build a phylogenetic tree, and an OTU table. For alpha and beta diversity, samples were rarified at a sequencing depth of 1000.

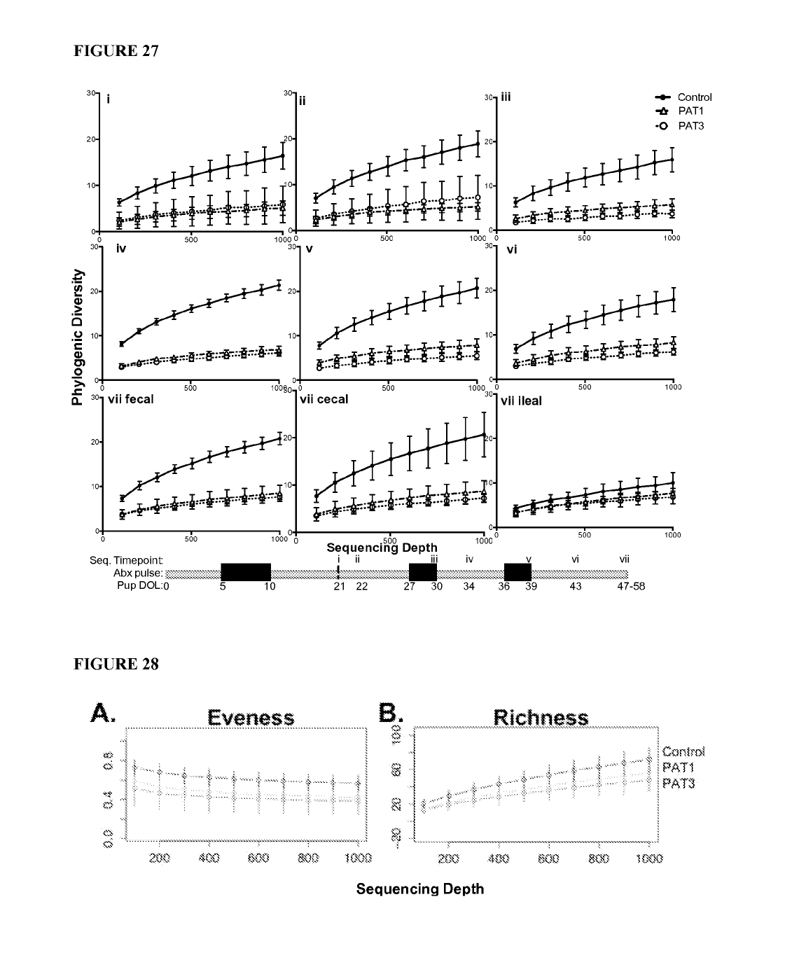

FIG. 27 shows alpha rarefaction plots of phylogenetic diversity in fecal, cecal and ileal samples in Control, PAT1 and PAT3 treated male pups over time. Fecal, cecal and ileal microbial diversity decreases with both one and three pulses of antibiotics and remain decreased throughout the life of the host. This indicates that early-life antibiotic treatment lead to long-term alterations in microbial diversity, which may have significant effects on alterations in Th17 and T-regulatory cell subsets.

Similar results of PAT on microbial diversity can be seen in FIGS. 28A and B, which show alpha rarefaction plots of microbial community evenness and richness in ileal Control, PAT1 and PAT3 at sacrifice. Ileal evenness (A) and richness (B) decreases with both one and three pulses of antibiotics at sacrifice, six-weeks and one-week after the last antibiotic pulse PAT1 and PAT3, respectively. This implies that one dose of an antibiotic, early-in-life, is sufficient to significantly alter ileal microbial communities, potentially impacting critical taxa involved in mucosal immunity and normal growth and development. This work provides a mechanism to identify candidate taxa that can be used to modulate systemic and mucosal immunity in animals and humans, as well as taxa, genera, and/or species of bacteria or other pathways that can be augmented to maximize immunological and metabolic health.

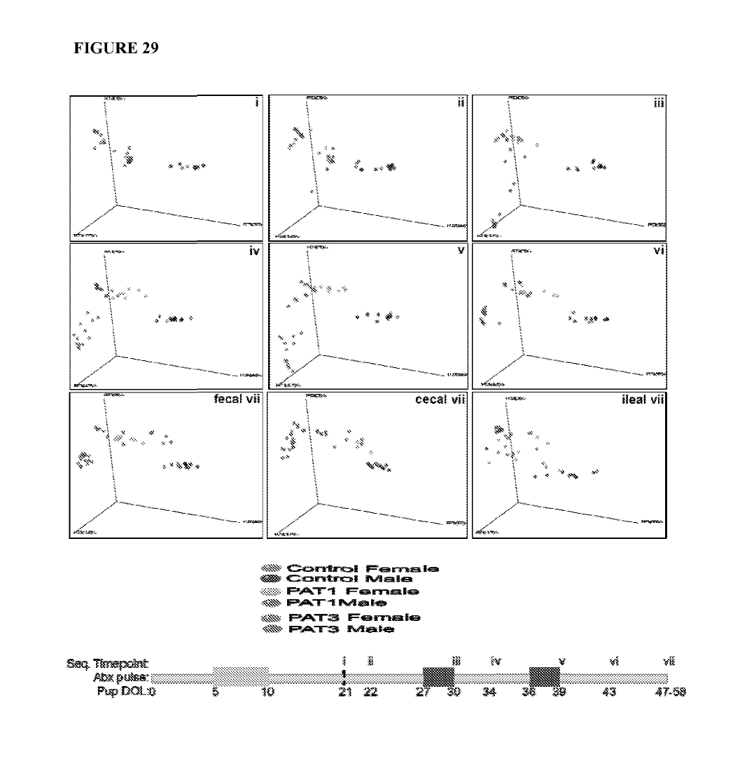

PAT occurs independently of sex, as demonstrated in FIG. 29, which shows Principal Coordinate Analysis (PCoA) of fecal, ileal and cecal samples in male and female Control, PAT1 and PAT3 treatment groups. Plots were generated using weighted Unifrac distance metric. The three components explain 56%, 8% and 4.2%, together these three components represent 68% of the total variation in the data. Both one and three therapeutic doses of Tylosin, early in life, significantly alters intestinal microbial populations in both males and females indicating that early-life antibiotic effects on microbial populations are independent of sex and may lead to alterations in host immunity.

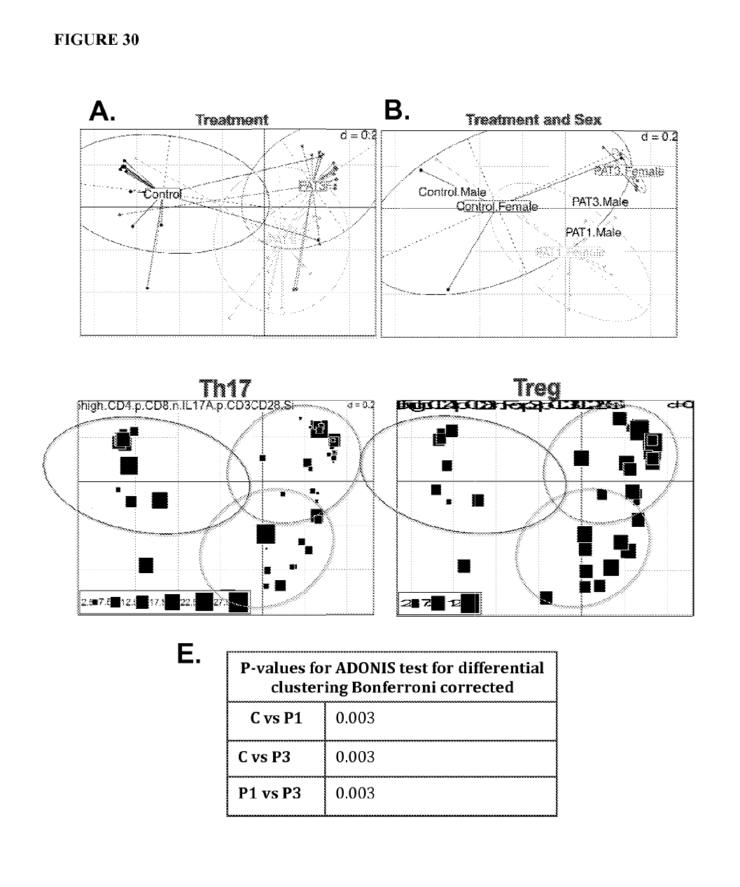

Results of further analysis of PAT on TH17 expression are depicted in FIGS. 30A-E and show Principal Coordinate Analysis (PCoA) of ileal microbial communities and ileal T-cell populations in male and female Control, PAT1 and PAT3 treatment groups. These plots were generated using the Jenson-Shannon divergence (JSD) distance metric to determine the similarity between Control, PAT1 and PAT3. PAT1 and PAT3 ileal microbial communities are significantly different from Control and from each other (as shown in panels A, C, E, respectively) independent of sex (B). Frequency of ileal Th17 populations greater than 12.5% cluster with Control samples (A). Contrastingly, the frequency of ileal Foxp3+ regulatory T cells (Tregs) greater than 12.5% cluster with both PAT1 and PAT3 groups. This data suggests that both one and three therapeutic doses of Tylosin, early in life, significantly alters intestinal microbial populations in both males and females, indicating that early-life antibiotic alters microbial populations and that these alterations are independent of sex. Additionally, this data implies that specific microbial taxa, such as Bifidobacterum, S24-7, and Akkermansia muciniphila may be involved in alterations of T-regulatory and Th17 populations.

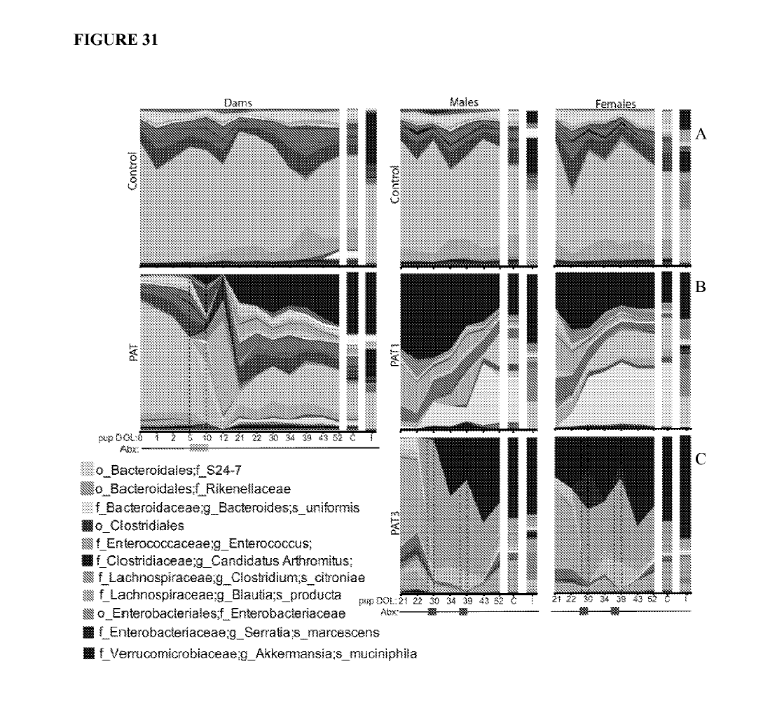

FIGS. 31A-C show the relative abundance of fecal, ileal and cecal microbial communities in dams and male and female offspring. Fecal samples were collected from nursing dams from birth of pups to sacrifice. PAT Dams were treated with one therapeutic dose of Tylosin 5 days post-birth of offspring for 5 days to indirectly treat offspring (PAT1). Control microbial communities were transferred from Dams to pups and remain relatively stable over time as depicted in Panel A. One therapeutic dose of tylosin altered both Dams' and offspring's microbial communities with blooms in Akkermansia muciniphila and Bacteroides uniformis and loss of Segemented Filamentous Bacteria, and remained altered in fecal, ileal and cecal samples seven weeks post antibiotic treatment, shown in Panel B. Further alterations in microbial communities occurred in the PAT3 group, which received two additional doses of the Tylosin antibiotic including blooms in the family Enterobacteriaceae and Blautia producta, shown in Panel C. This data indicates that early-life antibiotic treatment alters specific taxa and this alteration may lead to changes in the Th17/Treg populations.

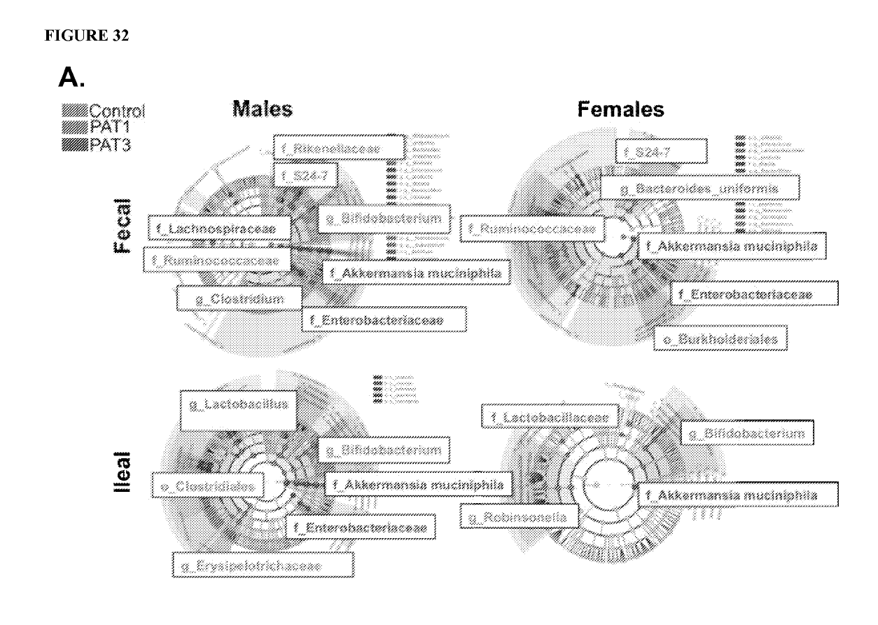

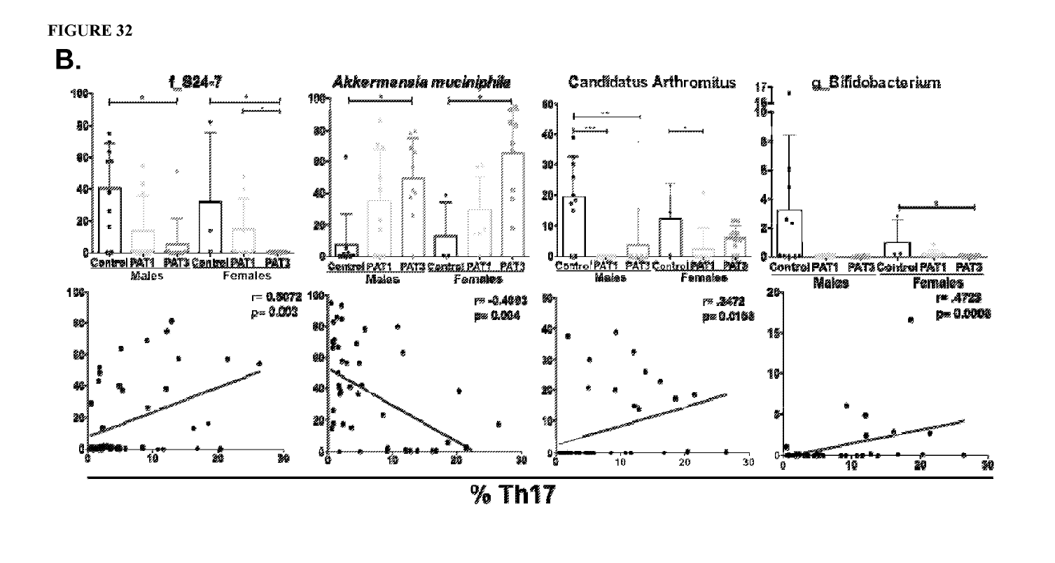

FIGS. 32A-B show differential microbial abundance of ileal microbial communities and association to Th17 population. Circular cladograms demonstrate significantly different taxa among treatment groups using the LEfSe module. Each color indicates the most abundant taxa in Control (light grey), PAT1, (darker grey) or PAT3 (darkest grey) groups. (A) Early-life PAT increases the relative abundance of Akkermansia muciniphila and decreases the abundance of S24-7, SFB and Bifidobacterium. Th17 cells are positively associated with S24-7, Bifidobacterium and Candidatus Arthromitus (SFB) and negatively associated with Akkermansia muciniphila (B). This data indicates taxa such as Bifidobacterum, S24-7, and Akkermansia muciniphila may be involved in alterations of Th17/Treg balance. Bifidiobacterum and S24-7 may influence the recruitment, differentiation, maturation, or phenotypic switch of Th17 cells. Akkermansia muciniphila may induce the recruitment, differentiation, maturation, or phenotypic switch of regulatory T cells.

### Example 5

### Acceleration and Promotion of Type 1 Diabetes (T1D)

Currently, there is the worldwide rise in type 1 diabetes (T1D), and it is occurring progressively earlier in life as it becomes more common. The present inventors explored whether antibiotic use might be triggering the rise, and, if so, whether the intermediary mechanisms could be identified. These questions were addressed in the NOD mouse model of T1D (Leiter, Curr Protoc Immunol 2001; Chapter 15:Unit 15 9; Atkinson M A, Nat Med 1999; 5:601-4).

Mice.

8-10 week old male and female NOD/Shiltj mice (Jackson Labs, Bar Harbor Me.) were bred in a specific pathogen-free (SPF) vivarium at NYUMC to obtain sufficient litters. Mice were fed irradiated laboratory chow (LabDiet J L Rat and Mouse/Irr 6F 5LG4) ad libitum.

Pregnant NOD/ShiLTj mice were randomized into 3 groups: Control, STAT, and PAT. Control mice were maintained on non-acidified sterilized water. STAT pregnant dams were given low dose penicillin beginning late in pregnancy until pups were 12 weeks of age. PAT mice were given 3 therapeutic doses of tylosin starting on day 10 of life. At 6 weeks of age, 13 male (5 control, 3 STAT, 5 PAT) and 15 female (5 per group) NOD mice were sacrificed for immune-phenotyping by flow cytometry and ileal gene expression analysis by microarray and qPCR. From 10 weeks of age to 31 weeks of age, mice were tested for diabetes weekly by blood glucose measurement and urinary glucose detection. Onset of diabetes was defined as two consecutive weeks of blood glucose >250 mg/dl and glucosuria.

Antibiotic Treatments.

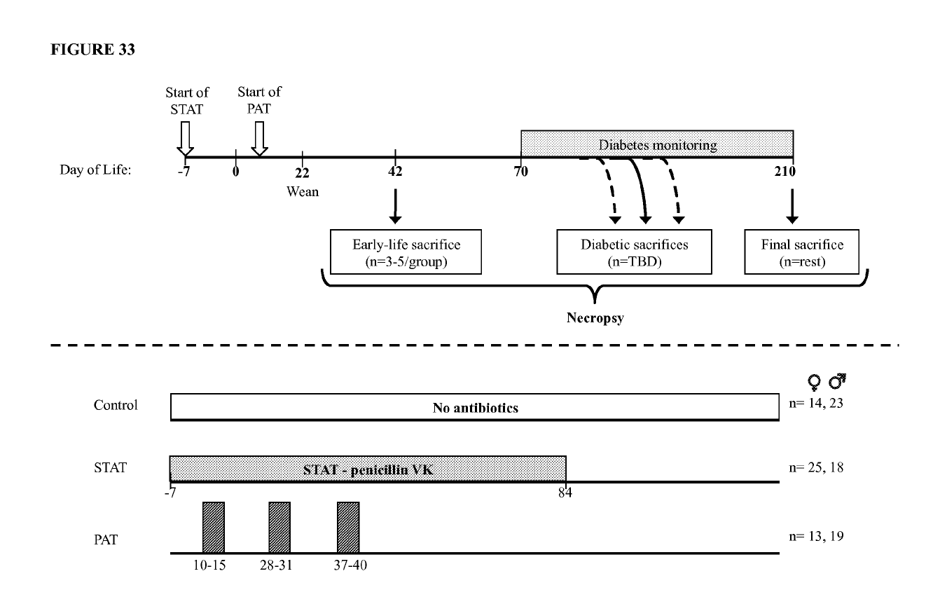

For the NOD experiments, dams (and their litters) were divided into control, STAT, and PAT groups (FIG. 33). The control mice were given non-acidified sterilized water throughout the experiments, and for the 2 antibiotic groups (STAT and PAT), the antibiotics were dissolved in non-acidified sterilized water. For STAT, subtherapeutic penicillin VK (1 mg/kg/day) was added to the drinking water in late pregnancy (so that the pups pass through an altered vaginal microbiota), and then continued until day 84 of life. For PAT, 3 pulses of therapeutic doses of the macrolide antibiotic tylosin (50 mg/kg/day) were given to the mice in their drinking water. The first pulse occurred pre-weaning, so pups received the antibiotics via their mother's milk. Mice were monitored for diabetes onset by glucosuria and blood glucose, starting at 10 weeks of age. Diabetes onset was defined as two consecutive weeks of glucosuria and blood glucose >250 mg/dl.

Isolation of Small Intestinal Lamina Propria Lymphocytes.

After euthanasia of the mice by CO₂ asphyxiation followed by cervical dislocation, the small intestines were dissected and immediately placed in cold calcium and magnesium-free DPBS, followed by removal of the mesenteric fat and Peyer's patches. The intestines were opened longitudinally and contents were removed by washing thoroughly with cold DPBS. The intestines were then cut into 2 cm pieces and treated with 10 ml 1 mM DTT/PBS, shaken at RT for 10 minutes to remove contaminating intraepithelial lymphocytes. Subsequently, the intestinal tissue was treated two times with 30 mM EDTA and 10 mM HEPES in PBS to remove epithelial cells. To isolate lamina propria lymphocytes, intestinal tissue was digested for 1.5 hours at 37° C. in digestion mix (0.5 mg/ml Collagenase/Dispase (Roche), 150 μg /ml DNase 1 (Sigma) in complete RPMI. Cells were passed through a 100 μm diameter nylon mesh filter (BD Bioscience). For further purification of leukocytes, intestinal cell suspensions were resuspended in 40% Percoll, under-layered with 80% Percoll (GE Healthcare Life Sciences) and centrifuged at 2,200 rpm for 25 minutes at room temperature. The leukocytes were collected at the interphase of the 40% 80% discontinuous Percoll gradient, then washed and re-suspended in DPBS for staining.

Lymphocyte Staining.

Cells were incubated with blue Live/DEAD fixable dye (Invitrogen) for 10 minutes at 4° C. to identify live cells. Intestinal leukocytes were phenotyped using the following antibodies: CD3-APC-Cy™7 (BD Biosciences), CD4− PE-Alexa Fluor 610 (Invitrogen), CD8− V500 (BD Biosciences), RORγt-PE (eBioscience), Foxp3-PE-Cy7 (eBioscience). Cells were first incubated with surface antibodies (each at 1:50 in FACS buffer) along with FC block (Anti-Mouse CD16/CD32, eBioscience) at 1:200 for 30 minutes at 4° C. Then the cells were fixed and permeabilized with Fix/Perm (eBioscience) and subsequently incubated with the nuclear antibodies in perm/wash buffer for 30 minutes at 4° C. (eBioscience).

Analysis of Lymphocyte Subsets.

All cells were acquired on a LSRII (BD Biosciences) and analyzed using FlowJo software (version 9.3.2; Tree Star Inc.).

Tissue Collection.

The distal ileum (1 cm) without Peyer's Patches was collected from each mouse for RNA extraction and kept in RNAlater (Ambion) overnight at 4° C. Samples were stored at −80° C. until RNA extraction was performed.

Gene Expression Analysis by Quantitative PCR (qPCR).

Total RNA was extracted from mouse tissues using the RNeasy Mini Kit with on-column DNase treatment to remove contaminating gDNA (Qiagen), according to the manufacturer's instructions. 1 μg of total RNA was reverse transcribed to cDNA using Super Script II (Applied Biosystems) according to the manufacturer's instructions. To generate standards for expression analysis of each target gene, the cDNA region of interest was PCR-amplified and the product cloned into pGEM-T easy (Promega). qPCR was performed with Power SYBR Green PCR Master mix (Roche) and run in a LightCycler 480 system (Roche). Target mRNA was normalized to 18S rRNA as an internal control in each sample. The primer sequences for qPCR are shown in Table 3.

TABLE 3
Primer sequences for NOD qPCR

| | | | SEQ | | | Anneal | Product | | Product |
|---|---|---|---|---|---|---|---|---|---|
| | | Sequence | ID | | Tm | T | (bp) | | (bp) |
| Gene | Orient. | (5′ to 3′) | NO | Location | (° C.) | (° C.) | gDNA | RNA | Reference |
| SAA1 | F | GAAGGAAGCTAACTG | 44 | exon 3-4, | 58.0 | 56 | 696 | 284 | (Son et |
| | | GAAAAACTC | | 159-442 | | | | | al. 2004) |
| | R | CAGGCCCCCAGCACA | 45 | | 66.5 | | | | |
| | | ACCTACT | | | | | | | |
| SAA2 | F | TGGCTGGAAAGATGG | 46 | exon 3-4, | 59.3 | 58 | 529 | 115 | Harvard |
| | | AGACAA | | 171-285 | | | | | Primer bank |
| | R | AAAGCTCTCTCTTGC | 47 | | 58.7 | | | | ID #6755394a1 |
| | | ATCACTG | | | | | | | (Spandidos |
| | | | | | | | | | et al. 2010) |
| SAA3 | F | TGCCATCATTCTTTG | 48 | exon 2-4, | 59.2 | 56 | 2854 | 248 | (Reigstad |
| | | CATCTTGA | | 49-276 | | | | | et al. 2009) |
| | R | CCGTGAACTTCTGAA | 49 | | 60.5 | | | | |
| | | CAGCCT | | | | | | | |
| 18S | F | CATTCGAACGTCTGC | 50 | 343-602 | 57.8 | 56 | 260 | 260 | (Snyder et |
| | | CCTAT | | | | | | | al. 2013) |
| | R | CCTCCAATGGATCCT | 51 | | 56.4 | | | | |
| | | CGTTA | | | | | | | |
| SFB | F | GACGCTGAGGCATGA | 52 | 722-830 | 62.8 | 62 | 109 | 109 | (Barman et |
| | | GAGCAT | | | | | | | al. 2009) |
| | R | GACGGCACGGATTGT | 53 | | 59.0 | | | | |
| | | TATTCA | | | | | | | |

Gene Expression Profiling.

Ileal gene expression profiling of 6-week control, STAT, and PAT animal groups, was performed using the Affymetrix Genechip system (Affymetrix, Santa Clara Calif.). Total RNA quality and quantity were determined using the Nanodrop ND-1000, agarose gel, and Agilent 2100 Bioanalyser. Total RNA (100 ng) was used to prepare cDNA following the Affymetrix 3′IVT Express Kit labeling protocol (Affymetrix). Standardized array processing procedures recommended by Affymetrix were performed, including hybridization, fluidics processing and scanning of the Affymetrix Mouse Genome 430 2.0 Array chips. The affy package in R was used to normalize the raw data (Affymetrix CEL files) by Robust Multichip Average (RMA) algorithm. Then the limma package was used to identify regulated genes between groups and generate lists of differentially expressed genes using linear models and empirical Bayes method with an FDR-adjusted p-value cut-off of p<0.05 (Smyth, 2004, Statistical Applications in Genetics and Molecular Biology 3, No. 1, Article 3).

Histology.

Pancreata from 6-week-old control, STAT and PAT mice were fixed in modified Bouin's fixative (17 ml saturated aqueous picric acid, 1 ml glacial acetic acid, 2 ml 38-40% formalin) and paraffin embedded, cut, and stained with Aldehyde Fuchsin for insulitis scoring (Leiter, Curr Protoc Immunol. 2001; Chapter 15:Unit 15 9). Five sections were made for each pancreas. Each of the sections is 5 microns thick with a 70 microns gap between sections. Insulitis was scored on a scale of 0-4: 0, normal islet; 1, peri-insular leukocyte aggregates; 2, leukocyte infiltration <25%; 3, leukocyte infiltration >25%, but <75%; 4, leukocyte infiltration >75% and β-cell destruction. For each mouse, an insulitis score was calculated based on the following formula: (total score for all islets)/(4×number of islets examined) (Forestier et al., Journal of immunology. 2007; 178, 1415).

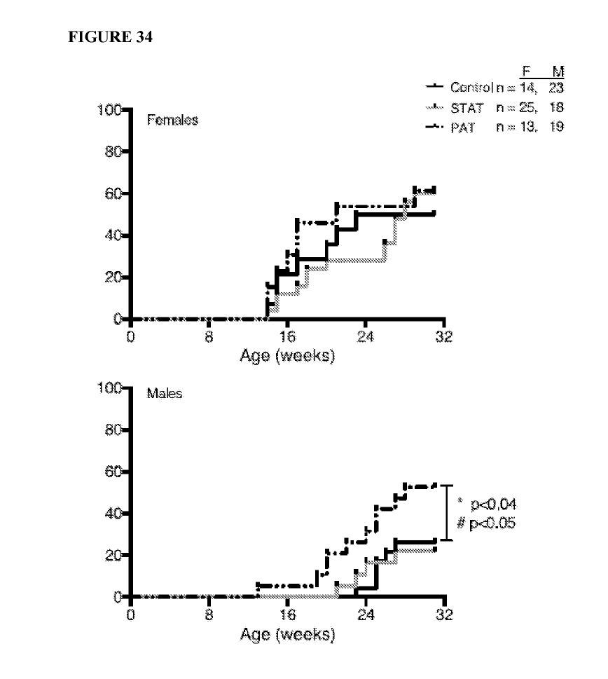

Diabetes onset was defined as two consecutive weeks with blood glucose >250 mg/dl and glucosuria. Diabetes incidence plots were compared using the log-rank test and the Gehan-Breslow-Wilcoxon test (# p<0.05, * p<0.04, respectively). As shown in FIG. 34, the PAT antibiotic exposure accelerated the development of T1D in the male NOD mice, consistent with our hypothesis. The finding indicates that the PAT antibiotic treatments that change the microbiota can change the likelihood of developing T1D.

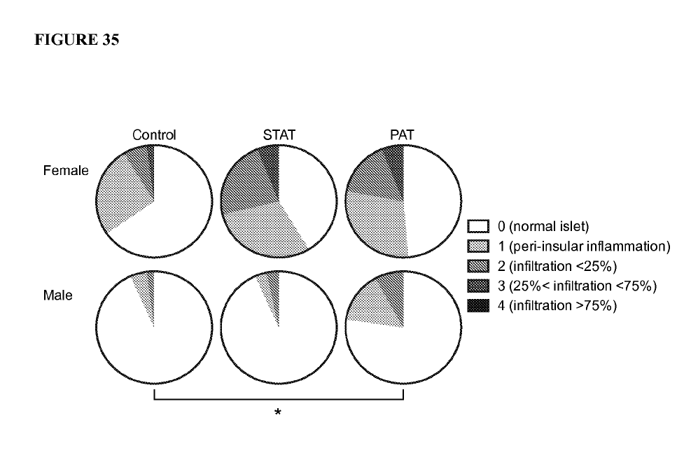

Upon necropsy, pancreata were preserved in modified Bouin's fixative, paraffin-embedded, and stained with aldehyde fuchsin, followed by a hemotoxylin and eosin counterstain. Five sections were made for each pancreas. Each of the sections is 5 microns thick with a 70 micron gap between sections. The most severely affected islet was scored from each section. Insulitis was scored on a scale of 0-4: 0, normal islet; 1, peri-insular leukocyte aggregates; 2, leukocyte infiltration <25%; 3, leukocyte infiltration >25%, but <75%; 4, leukocyte infiltration >75% and β-cell destruction, according to standard criteria. For the females, we scored at least 24 islets per group (5 mice per group). STAT females had significantly more islet inflammation than controls (FIG. 35). For the males, 25 islets were scored for the control and PAT group (5 mice per group), and 15 islets from the STAT group (3 mice). PAT males had significantly more insulitis than control or STAT males (FIG. 35). The Kruskal-Wallis test with Dunn's multiple comparison correction was used to determine group differences, *p<0.05. C=control, S=STAT, and P=PAT. These results provide evidence for the early development of insulitis in the PAT male mice at 6 weeks of age, many weeks before the development of overt diabetes, and are consistent with the observations in FIG. 34 of earlier diabetes onset on average in the mice receiving PAT.

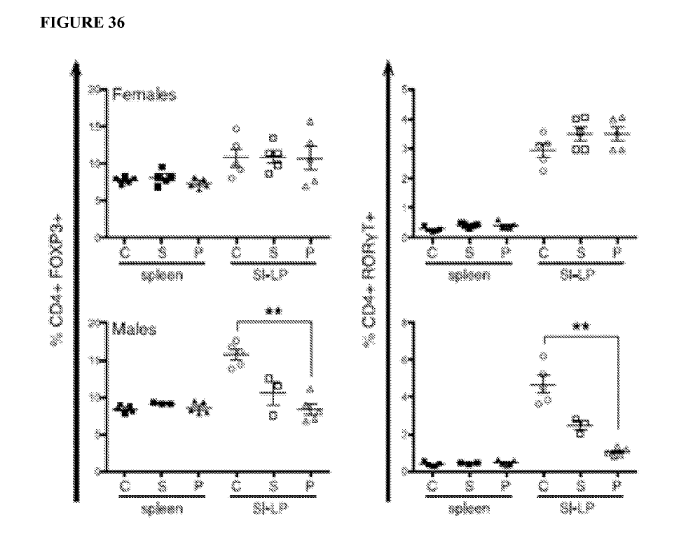

Flow cytometry was performed on splenic and SI-LP cells from 6-week old male and female NOD mice. CD3 and CD4 surface stains and a live/dead stain were used to gate on live T-helper cells. Nuclear staining for FOXP3 and RORγT was performed to identify Treg and Th17 cells, respectively. By qPCR, SFB was below detectable limits in fecal specimens collected at 3 weeks and 6 weeks of age from all of the mice used in the above analysis. The range for SFB qPCR assay was from 10² to 10⁶ copies of SFB. FIG. 36 shows quantitation of Treg and Th17 cells isolated from the spleen and SI-LP. PAT decreased the percent of SI-LP Treg and Th17 cells in male, but not female NOD mice. C=controls, S=STAT, P=PAT. Statistical analysis was performed on each tissue separately, using the Kruskal-Wallis test followed by the Dunn's multiple comparison test, ** p≦0.01. The data in FIG. 36 show that the mice receiving PAT had significantly lower percent of Treg and Th17 CD4+ cells than the controls, and that the mice receiving STAT were intermediate. This work provides evidence that PAT changes T-cell populations in the NOD mice that are developing T1D and insulitis earlier.

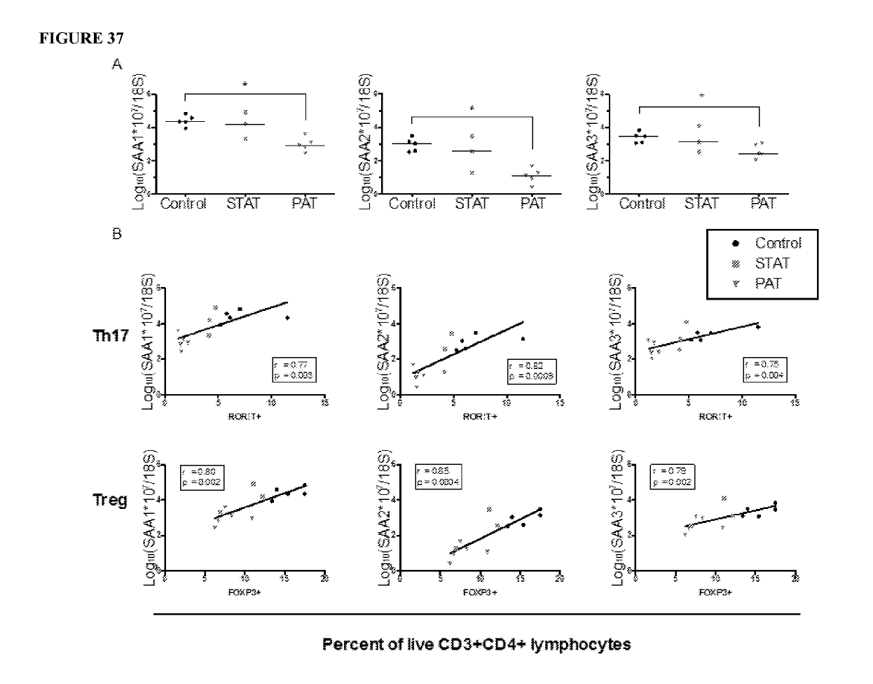

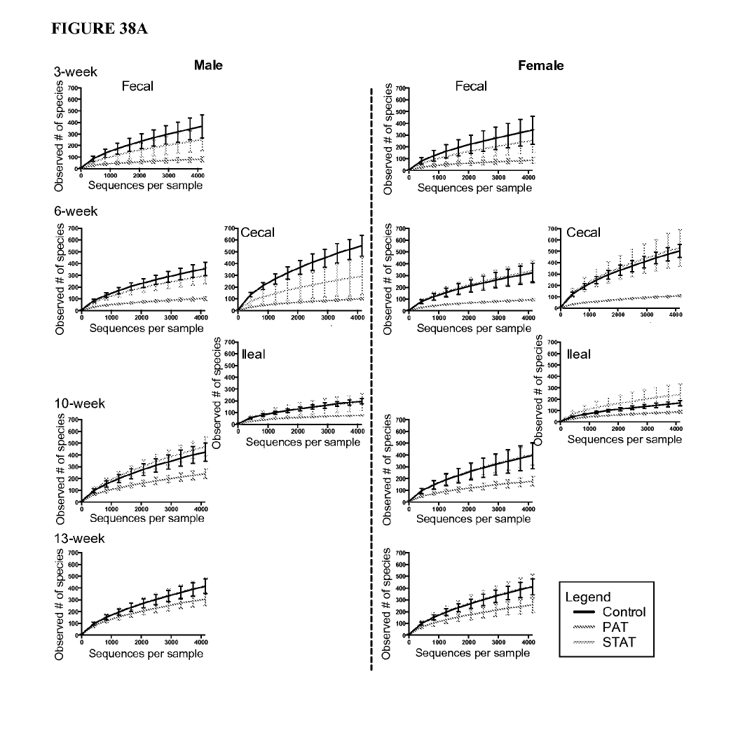

RNA was extracted from the terminal ileum of the 6-week old NOD mice. FIG. 37A shows the expression of SAA 1, 2, and 3 gene expression by qPCR. RNA was reverse transcribed into cDNA and then qPCR was performed for SAA1, 2, or 3 using an absolute quantitation method. Gene expression data were normalized to 18S rRNA expression. Statistical analysis was performed using the Kruskal-Wallis test, followed by Dunn's multiple comparison test, * p<0.05. The results for this experiment show consistent decreases in expression of the SAA genes in the ileum, detected by two independent methods (microarray and qPCR). Since the SAA protein is a strong signal for the development of Th17 cells, this experiment provides a way to link intestinal microbiota peruturbation with T helper cell differentiation. The work in this experiment provides indications that ways to manipulate host immunity using SAA or other differentially expressed genes via the microbiota would have utility for manipulating immune responses.

RNA was extracted from the terminal ileum of the 6-week old NOD mice. Raw probe intensities were normalized by the Robust Multichip Average (RMA) algorithm using affy package in R and then the limma package was used to identify regulated genes using linear models and empirical Bayes method with an FDR-adjusted p-value cut off of p<0.05 (Smyth 2004, Statistical Applications in Genetics and Molecular Biology 3, No. 1, Article 3.). Select up-regulated and down-regulated genes in PAT-treated male NOD mice compared to control males are shown in Tables 4 and 5, respectively.

TABLE 4
Genes up-regulated in terminal ileum of PAT-treated NOD
male mice compared with controls (by microarray)

| | | log₂ fold | | FDR | | |
|---|---|---|---|---|---|---|
| Gene | | change | | adjusted | Accession number | |
| Symbol | Gene Name | (PAT/control) | p value | p value | Mus musculus | Homo sapiens |
| Rab30 | RAB30, member RAS | 4.27 | 6.70E−10 | 0.00002 | NM_029494.2 | NM_014488.3 |
| | oncogene family | | | | | |
| Areg | amphiregulin | 3.94 | 4.92E−06 | 0.003 | NM_009704.3 | NM_001657.2 |
| Flrt3* | fibronectin leucine rich | 2.56 | 1.08E−05 | 0.004 | NM_001172160.1 | NM_013281.3 |
| | transmembrane protein 3 | | | | | |
| Arl5b | ADP-ribosylation factor-like | 2.42 | 4.36E−05 | 0.008 | NM_029466.4 | NM_178815.3 |
| | 5B | | | | | |
| Maff | v-maf musculoaponeurotic | 2.22 | 5.52E−07 | 0.001 | NM_010755.3 | NM_012323.3 |
| | fibrosarcoma oncogene | | | | | |
| | family, protein F (avian) | | | | | |
| Dusp14 | dual specificity phosphatase | 2.22 | 5.99E−06 | 0.004 | NM_019819.3 | NM_007026.2 |
| | 14 | | | | | |
| Zfand2a* | zinc finger, AN1-type domain | 2.11 | 2.57E−06 | 0.002 | NM_133349.3 | NM_182491.2 |
| | 2A | | | | | |
| Chka* | choline kinase alpha | 1.97 | 1.72E−07 | 0.001 | NM_013490.4 | NM_001277.2 |
| Phlda1 | pleckstrin homology-like | 1.96 | 1.13E−05 | 0.005 | NM_009344.3 | NM_007350.3 |
| | domain, family A, member 1 | | | | | |
| Ereg | epiregulin | 1.73 | 1.05E−06 | 0.002 | NM_007950.2 | NM_001432.2 |
| Tnfrsf12a* | tumor necrosis factor receptor | 1.72 | 1.69E−06 | 0.002 | NM_013749.2 | NM_016639.2 |
| | superfamily, member 12a | | | | | |
| Il1rn | interleukin 1 receptor | 1.70 | 1.22E−03 | 0.046 | NM_031167.5 | NM_173842.2 |
| | antagonist | | | | | |
| Il1rl1* | interleukin 1 receptor-like 1 | 1.64 | 1.64E−04 | 0.017 | NM_001025602.2 | NM_016232.4 |
| Acsl3* | acyl-CoA synthetase long- | 1.59 | 1.16E−04 | 0.014 | NM_028817.3 | NM_004457.3 |
| | chain family member 3 | | | | | |
| Slc2a1* | solute carrier family 2 | 1.26 | 4.00E−04 | 0.027 | NM_011400.3 | NM_006516.2 |
| | (facilitated glucose | | | | | |
| | transporter), member 1 | | | | | |
| S100a14 | S100 calcium binding protein | 1.00 | 7.75E−04 | 0.038 | NM_001163525.2 | NM_020672.2 |
| | A14 | | | | | |
| Klf4* | Kruppel-like factor 4 (gut) | 0.86 | 9.71E−04 | 0.041 | NM_010637.3 | NM_004235.4 |
| Gjb3 | gap junction protein, beta 3 | 0.77 | 4.46E−07 | 0.001 | NM_001160012.1 | NM_024009.2 |

Bolded gene symbol indicates that the gene is up-regulated in PAT-treated females as well as PAT-treated males.
*indicates that multiple probes for that gene are significantly up-regulated in PAT-treated males. If there are multiple significant probes, fold change and p values listed are only for the probe with greatest fold change.

TABLE 5
Genes down-regulated in terminal ileum of PAT-treated NOD
male mice compared with controls (by microarray)

| | | log₂ fold | | FDR | | |
|---|---|---|---|---|---|---|
| Gene | | change | | adjusted | Accession number | |
| Symbol | Gene Name | (PAT/control) | p value | p value | Mus musculus | Homo sapiens |
| Saa1 | serum amyloid A 1 | −4.00 | 1.68E−05 | 0.006 | NM_009117.3 | NM_000331.4 |
| G6pc | glucose-6-phosphatase, | −2.85 | 4.39E−07 | 0.001 | NM_008061.3 | NM_000151.3 |
| | catalytic | | | | | |
| Edn2 | endothelin 2 | −1.67 | 1.19E−07 | 0.001 | NM_007902.2 | NM_001956.3 |
| Bhmt | betaine-homocysteine | −1.52 | 1.28E−04 | 0.015 | NM_016668.3 | NM_001713.2 |
| | methyltransferase | | | | | |
| Gzma | granzyme A | −1.48 | 2.56E−04 | 0.021 | NM_010370.2 | NM_006144.3 |
| Tnfsf10* | tumor necrosis factor | −1.46 | 1.21E−07 | 0.001 | NM_009425.2 | NM_003810.3 |
| | (ligand) superfamily, | | | | | |
| | member 10 | | | | | |
| Saa2 | serum amyloid A 2 | −1.44 | 8.54E−04 | 0.039 | NM_011314.2 | NM_030754.4 |
| Tppp | tubulin polymerization | −1.35 | 3.28E−04 | 0.024 | NM_182839.2 | NM_007030.2 |
| | promoting protein | | | | | |
| Dio1 | deiodinase, iodothyronine, | −1.34 | 1.05E−03 | 0.042 | NM_007860.3 | NM_000792.5 |
| | type I | | | | | |
| Tifa | TRAF-interacting protein | −1.32 | 6.44E−05 | 0.011 | NM_145133.3 | NM_052864.2 |
| | with forkhead-associated | | | | | |
| | domain | | | | | |
| Irf1 | interferon regulatory | −1.14 | 2.20E−05 | 0.007 | NM_008390.2 | NM_002198.2 |
| | factor 1 | | | | | |
| Cd38* | CD38 antigen | −1.02 | 3.48E−05 | 0.008 | NM_007646.4 | NM_001775.2 |
| Ccl20 | chemokine (C-C motif) | −0.86 | 3.07E−04 | 0.023 | NM_016960.2 | NM_004591.2 |
| | ligand 20 | | | | | |
| Socs2* | suppressor of cytokine | −0.77 | 8.65E−06 | 0.004 | NM_007706.4 | NM_003877.4 |
| | signaling 2 | | | | | |
| Clec2d | C-type lectin domain | −0.65 | 1.55E−04 | 0.017 | NM_053109.3 | NM_013269.5 |
| | family 2, member d | | | | | |
| Ccl28* | chemokine (C-C motif) | −0.64 | 1.09E−04 | 0.014 | NM_020279.3 | NM_148672.2 |
| | ligand 28 | | | | | |
| Il17rd | interleukin 17 receptor D | −0.58 | 1.44E−04 | 0.016 | NM_134437.3 | NM_017563.3 |

Bolded gene symbols indicates that the gene is down-regulated in PAT-treated females as well as PAT-treated males.
*indicates that multiple probes for that gene are significantly down-regulated in PAT-treated males. If there are multiple probes, fold change and p values listed are only for the probe with greatest fold change

SAA 1, 2, and 3 ileal gene expression was determined by reverse transcriptase qPCR. The percent of Th17 and Treg cells was determined by flow cytometry. Spearman's correlation was used to determine whether there was a significant correlation between SAA 1, 2, and 3 gene expression and the percent of Th17 and Treg cells. The results shown in FIG. 37B provide further indication of the linkage between SAA and Treg and Th17 cell proportions in the ileum, and provide further evidence for a role of manipulating SAA in the ileum as a way of perturbing ileal T cell populations.

NOD Gut Microbiome Sample Collection and DNA Extraction.

Fecal pellets were collected at least weekly from weaning at 3 weeks of age until the study termination at 31 weeks of age, and stored at −20° C. until processing. DNA was extracted from fecal pellets using the PowerLyzer™ PowerSoil® DNA Isolation Kit (MoBio, Carlsbad Calif.) and stored at −20° C. Cecal and ileal samples were collected upon necropsy at 6 weeks of age, upon diabetes development, or at the termination of the experiment at 30 weeks of age, and extracted using the PowerLyzer™ PowerSoil® DNA Isolation Kit.

Amplicon Amplification and Sequencing of V4 Region of 16S rRNA Genes.

Fecal samples collected at 3, 6, 10, and 13 weeks of age, and cecal and ileal samples collected at 6 weeks of age were used for sequencing. The V4 region of the 16S rRNA gene was amplified using universal primers 515 forward primer (5′ GTGCCAGCMGCCGCGGTAA 3′ SEQ ID NO: 54) and 806 reverse primer (5′ GGACTACHVGGGTWTCTAAT 3′ SEQ ID NO: 55) [Escherichia coli strain 83972 sequence (greengenes accession no. prokM-SA_id:470367] linked to a 12 base Goley barcode for multiplexing (Caporaso et al., The ISME Journal 2012; 6:1621-4. Caporaso et al., Proc. Natl. Acad. Sci. USA, 2011; 108 Suppl 1:4516-22). Sequencing was performed on the Miseq platform with a 15% PhiX spike.

Analysis of 16S rRNA Sequencing.

The sequences were analyzed using the macqiime 1.7.0 software (Caporaso et al., Nature Methods 2010; 7:335-6). Sequences were aligned using the Ribosomal Database Project (RDP) classifier (Wang et al., Applied and Environmental Microbiology 2007; 73:5261-7). Taxonomy was assigned based on the GreenGenes May 2013. For alpha diversity measurements, observed number of species and phylogenic distance (PD) were calculated. For beta diversity measurements, unweighted unifrac distances were calculated (Lozupone C, Knight R., Applied and Environmental Microbiology 2005; 71:8228-35). The unweighted unifrac distances were used for principal coordinates analysis (PCoA), and visualized using the KiNG software. Further sequencing analysis and correlations were performed using the phyloseq and ellipse packages in R.

The present inventors also used the LEfSe algorithm developed by Curtis Huttenhower and colleagues (Segata et al., Genome Biology 2011, 12:R60) to identify key taxa that have significantly different relative abundances according to treatment group or sex.

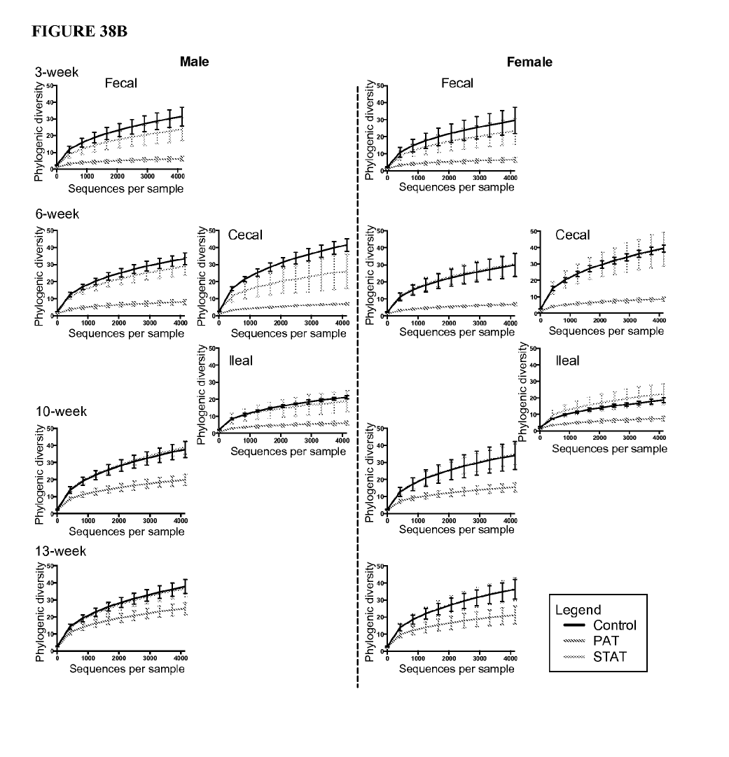

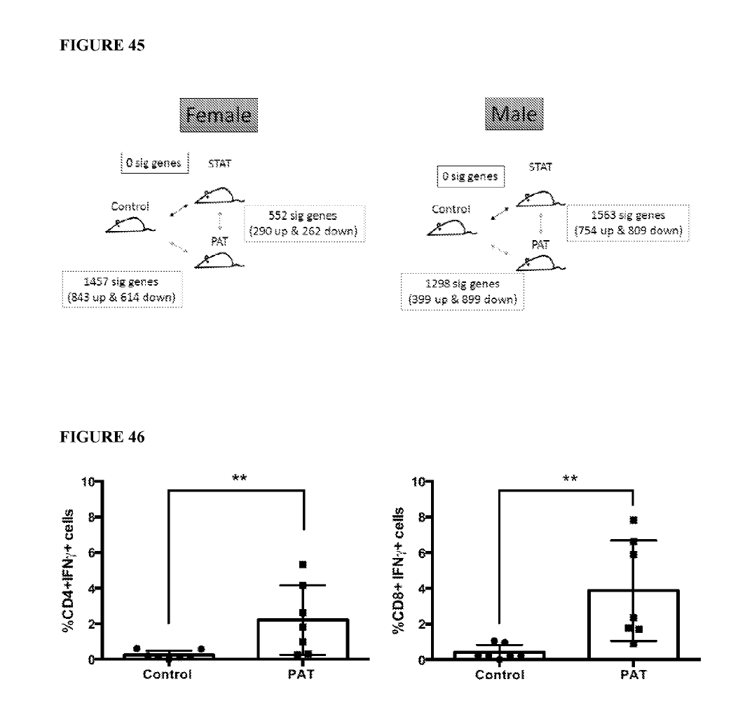

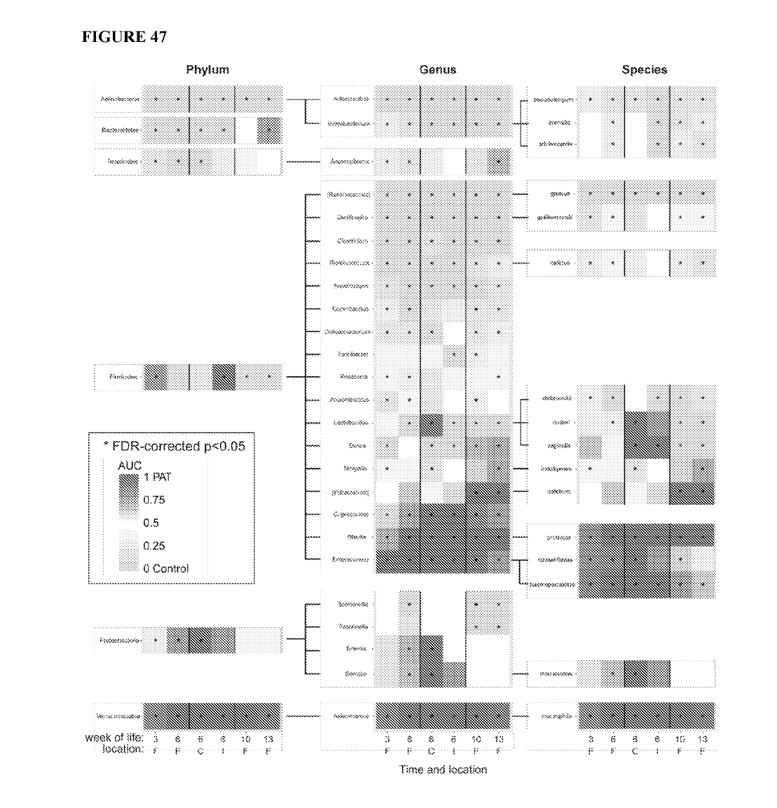

FIGS. 38-44 show various characteristics of the microbiome in NOD mice. FIG. 45 depicts an overview of significant ileal genes among different treatment groups. FIG. 46 shows that PAT-treated mice have greater systemic inflammation as evidenced by the increase in production of pro-inflammatory cytokine IFNγ by both CD4 and CD8 T cells. PAT also decreases Bifidobacterium and thus, Bifidobacterium are believed to help dampen the production of this pro-inflammatory cytokine.

FIGS. 38A-B show that PAT decreases richness in fecal, cecal, and ileal samples from male and female NOD mice. Fecal samples from control, PAT-, and STAT-exposed male and female mice at 3, 6, 10, and 13 weeks of age, and cecal and ileal samples from 6-week old mice were studied. DNA was extracted and the V4 region of the 16S rRNA gene sequenced and analyzed. Samples (n=555) were rarefied to a depth of 4150, which only excluded 5 samples from the analysis. (A) Alpha rarefaction plots of observed OTUs. At all sample times, in all sample types, and in both males and females, OTU richness is lower in PAT-exposed mice compared to controls (FDR corrected p-value <0.05). However, after the final PAT pulse ending on day 39 of life, OTU richness partially recovered by 13 weeks of age. In males, STAT has significantly lower OTU richness than controls at 3 and 6 weeks of age, and in females, STAT has significantly lower richness than controls only at 3 weeks of age (FDR corrected p-value <0.05). In all other comparisons, there were no significant differences between STAT and controls, despite continued antibiotics until 13 weeks of age. Compared to fecal control samples, OTU richness was significantly higher for cecal control samples and lower for ileal samples (FDR corrected p-value <0.05). (B) Phylogenic diversity (PD). At all sample times, in all sample types, and both in males and females, PD is lower in PAT compared to controls (FDR corrected p-value <0.05). However, after the last PAT pulse, PD is partially recovered. Relationships with STAT, and for cecal and ileal samples are essentially as in (A) for OTU richness, further confirming the impact of PAT on the intestinal microbial community.

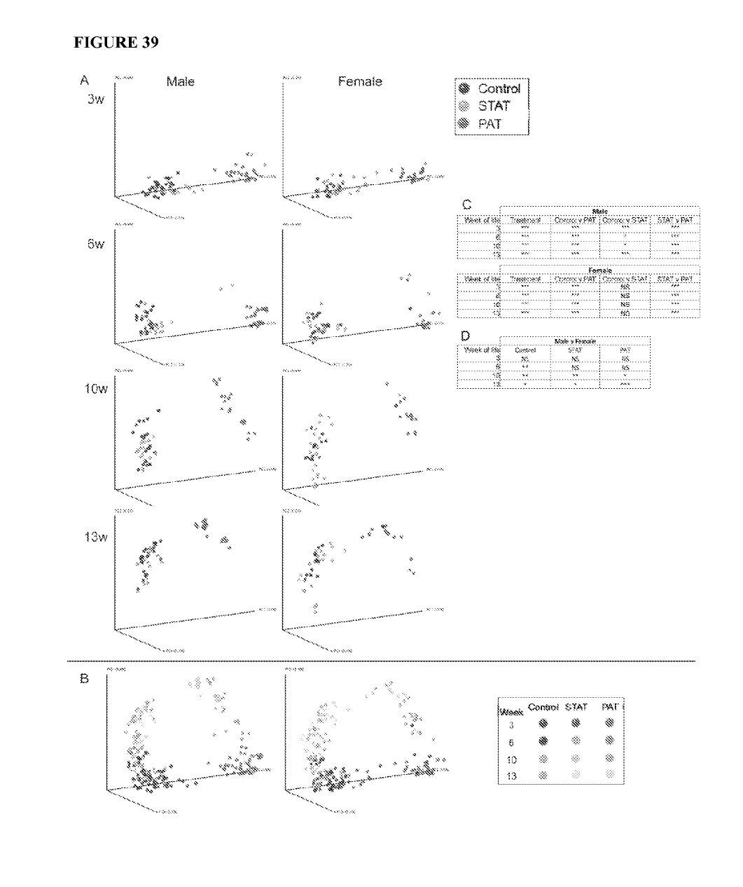

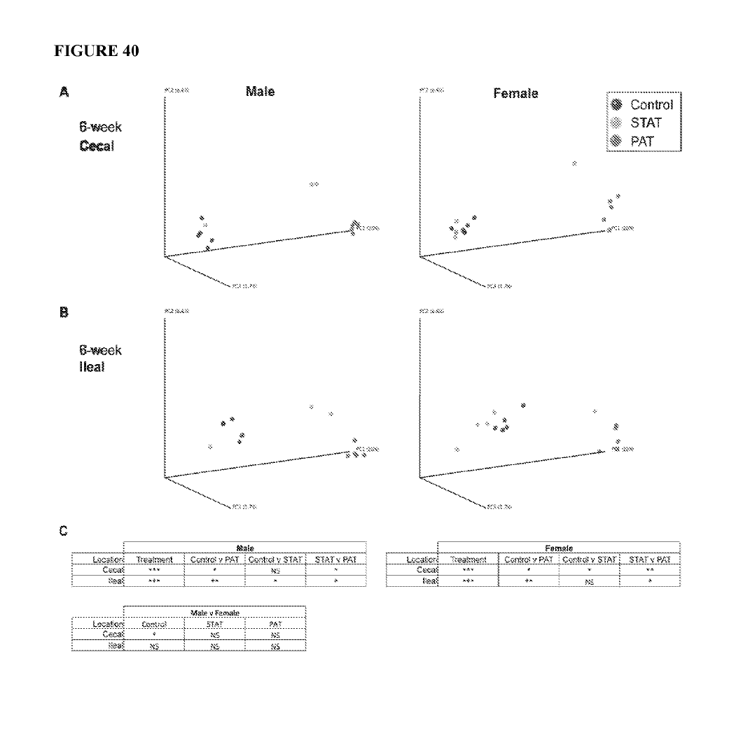

FIGS. 39 and 40 show that PAT differentially alters intestinal microbial community structure in male and female NOD mice. (39A) Fecal microbiota at each timepoint. At all time points examined and in both males and females, PAT samples (medium grey circles) cluster distinctly from controls (black circles). Control and STAT samples essentially overlap at all time points. The difference with PAT is chiefly represented on Principal Component 1 (PC 1), which by definition accounts for the most diversity in this set of samples. From 3 to 13 weeks of age, the representation of all samples moves up on the PC2 axis and ultimately closer together on the PC 1 axis, although PAT remains distinct. (39B) Dynamic changes in the fecal microbiome. All time points of fecal samples displayed on a single set of axes. For 39C and 39D, the Adonis (non-parametric multivariate analysis of variance) test in R was used to determine whether the separation between groups is statistically significant, *p<0.05, **p<0.01, ***p<0.001. (39C) The effect of treatment on the grouping within each sex. (39D) The effect of sex within each treatment group. These results suggest that PAT has long term effects on the fecal microbiota composition. Additionally, the microbial composition becomes distinct by sex beginning at 6-weeks of age. The developmental phenotype of early diabetes persists, thus supporting the idea that early changes in the microbial community in the intestine are important to growth and development.

FIGS. 40 A-C show Cecal (A) and ileal (B) microbial community structure in 6-week old mice. PAT clusters distinctly from control and STAT. STAT has a partial effect in the males, with some samples clustering near controls and others near PAT. The Adonis test (C) was used to determine if the separation between groups is statistically significant,*p<0.05, **p<0.01, ***p<0.001. Consistent with the fecal samples, PAT significantly alters both cecal and ileal microbial composition.

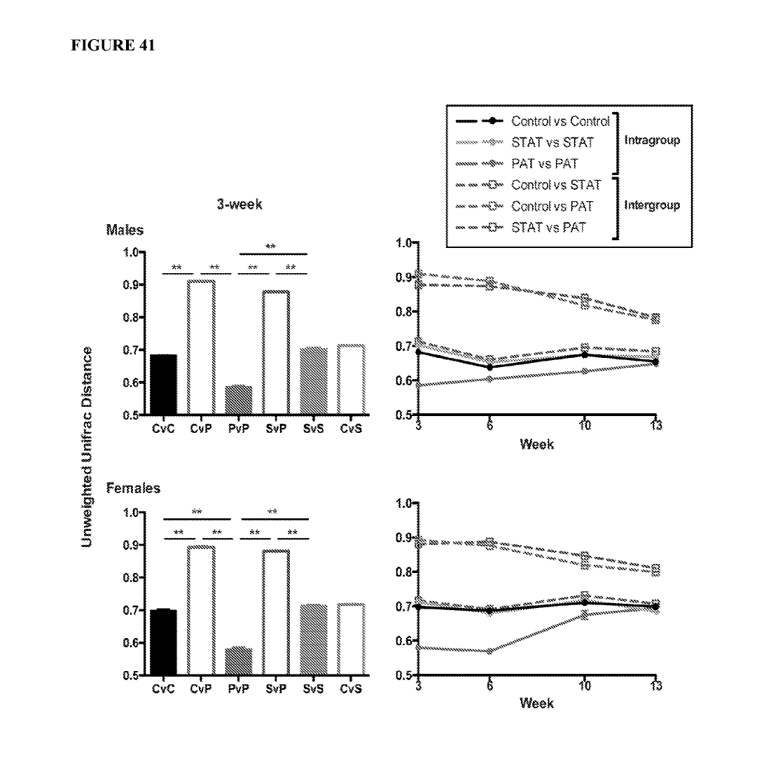

FIG. 41 shows beta diversity as measured by unweighted Unifrac differs in PAT-treated mice. Beta diversity, as measured by unweighted Unifrac analysis of samples from control, STAT-, and PAT-treated mice. In male mice, intragroup beta diversity is similar from 3 to 13 weeks of life in control mice. In STAT-treated mice, intragroup beta diversity is similar to that of controls. STAT distance from controls is similar to control intragroup distance. PAT-treated mice have lower intragroup distance than controls early in life. Distances from controls to PAT-treated mice is greater than control intragroup distance. Results in female mice are parallel to those for male mice Statistical significance of the inter- and intra-group beta diversity was determined by permutation testing. The p-value was calculated using the fraction of the random permutations out of 10,000 that yielded a greater difference than in the one observed (t-statistic), followed by Bonferroni's correction for multiple comparisons *p<0.01, ** p<0.01. As in FIG. 40, this figure suggests that PAT significantly alters the microbial composition with some recovery over time.

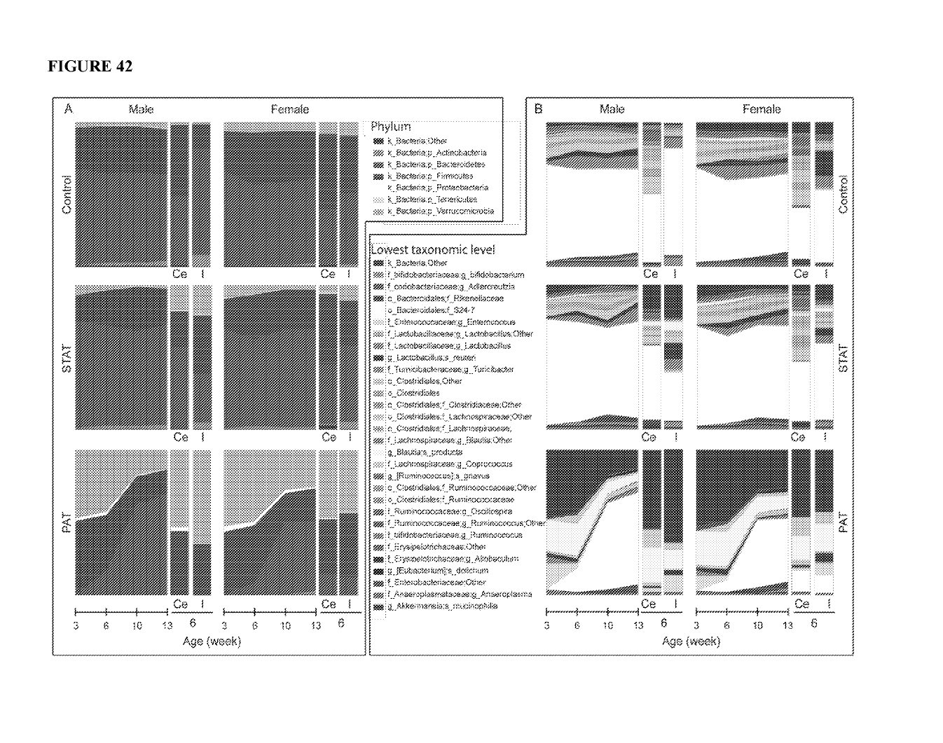

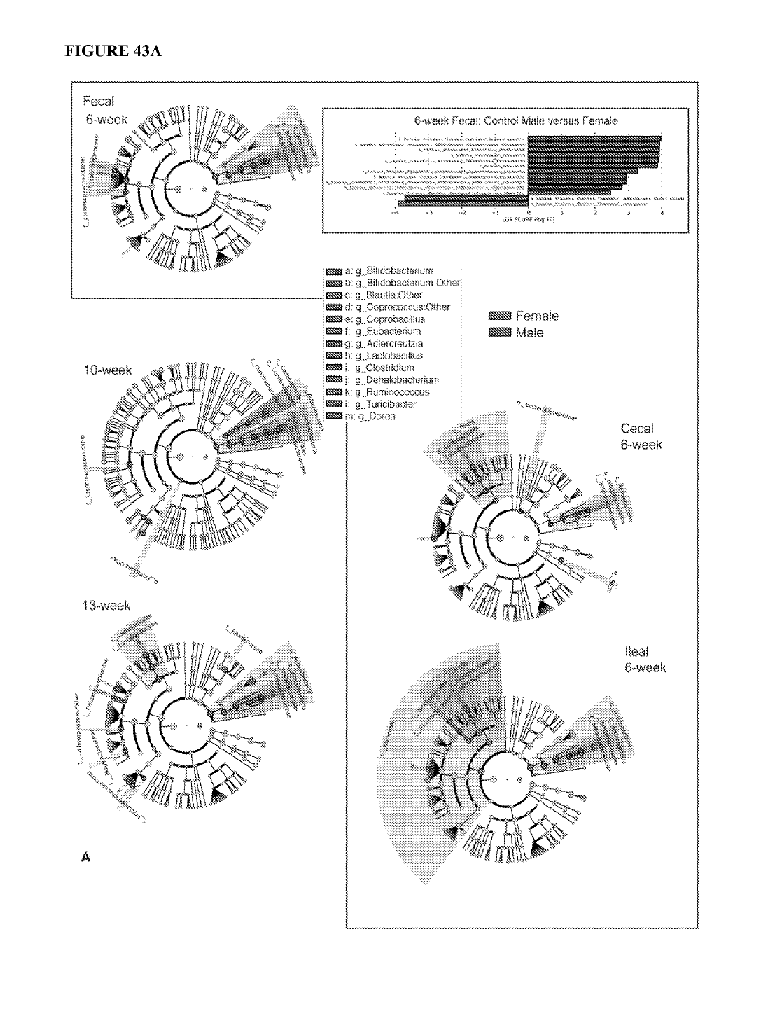

FIGS. 42A-B show taxonomy of fecal, cecal, and ileal samples. 16S rRNA sequencing of the V4 region was performed on fecal, cecal, and ileal samples from male and female NOD mice. (A) Phylum level. Male and female control samples have similar compositions over time. Among the control cecal and ileal samples, males have less Verrucomicrobia than females. STAT fecal samples appear very similar to controls. In cecal and ileal samples from male STAT mice there is a bloom in Verrucomicrobia greater than in controls but less than in PAT. After the first antibiotic pulse, the PAT samples show a large bloom in Verrucomicrobia not seen in control mice, and decrease in Bacteroides. The PAT samples become more similar to controls over time but do not recover completely by 13 weeks (7 weeks after the last antibiotic exposure). Cecal and ileal samples follow the same trends as the fecal samples with some differences in the STAT males. These results suggest that PAT has a greater effect on the microbial composition as compared to STAT. There is some recovery after cessation of the antibiotics but this is not complete. (B) Species level. Results parallel those observed at the phylum level. STAT male cecal and ileal samples have more Akkermansia muciniphila (a species within the Verrucomicrobia phylum) than controls, but less than that in PAT samples. PAT samples show a large bloom in A. muciniphila starting at 3 weeks (after the first antibiotic pulse); the proportions of A. muciniphila decrease over time but remain greater than that seen in controls. These taxa differences may be associated with the risk for diabetes onset, particularly Type 1 diabetes onset.

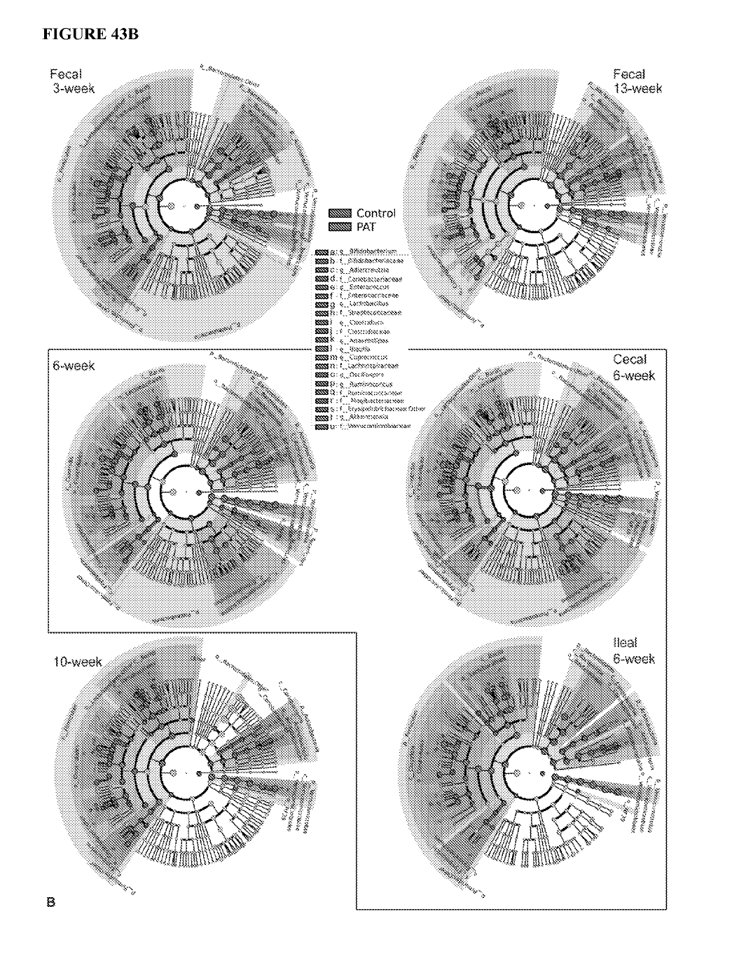

FIGS. 43A-B show identification of taxa differences by group using LEfSe. From each sample, OTU relative abundances were analyzed using the LEfSe algorithm to identify differentially abundant taxa with reference to treatment group or sex. The figures shown are cladograms depicting these differences and their phylogenic relationships. The colors indicate that the taxon is significantly enriched. The taxa enriched in control males relative to control females as well as in control males relative to PAT males can be thought of as diabetes protectors; while those enriched in either control female samples or PAT male samples relative to control males may be thought of as diabetes accelerators. The size of the individual taxon circle correlates with the relative abundance of that taxon. (A) Comparison of male and female control NOD mice. Fecal microbial communities from male and female NOD mice were compared using LEfSe at weeks 3, 6, 10, and 13 of life. The darker grey color indicates that the taxon is significantly enriched in males while the lighter grey color indicates that the taxon is significantly enriched in females. At week 3 (pre-pubertal), the taxa in males and females were similar. At week 6, 10, and 13 of life, consistent taxa are over-represented in male mice. Furthermore, 6-week cecal and ileal samples from males show the same enriched taxa. Key taxa from the Actinobacteria phylum including the genus Bifidobacterium are consistently identified as enriched in males in our study before the development of diabetes. Since males are relatively protected from diabetes compared to females, we hypothesize that these may be key protective taxa and plan to follow up on these studies. (B) Comparison of male control and PAT mice. Similar comparisons were made in male mice either in the PAT or control group to determine the key differential taxa. The darker grey color indicates that the taxon is significantly enriched in control males while the lighter grey color indicates that the taxon is significantly enriched in PAT males. As in the male to female comparison, control male mice are relatively protected from diabetes development compared to PAT male mice, therefore taxa identified as enriched in male controls are hypothesized to be key protective taxa. Consistent with the male and female comparison data, we find that taxa from Actinobacteria phylum and more specifically the genus Bifidobacterium are enriched in male controls relative to PAT males. These differences are present in fecal samples at 6, 10, and 13 weeks of life as well as cecal and ileal samples from 6 weeks of life. At 3 weeks of life, the overall phylum of Actinobacteria is significantly greater in controls, but the genus Bifidobacterium does not reach statistical significance.

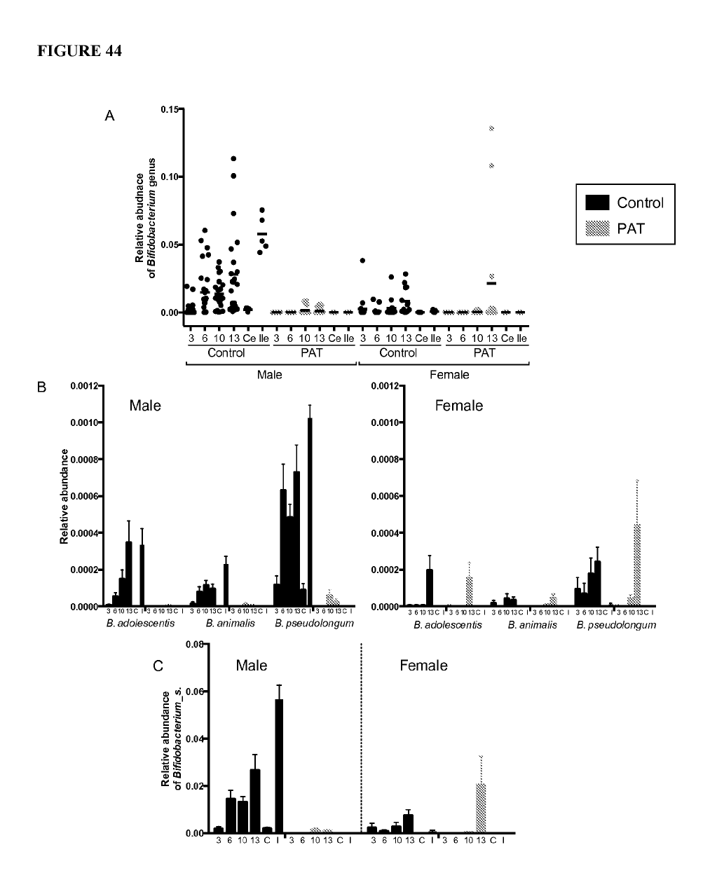

FIG. 44 shows taxa with significantly different abundances in control and PAT male NOD mice across timepoints and locations. Area under the curve analysis was used to determine significantly different taxa, with FDR correction for multiple comparisons. Taxa with at least two significant timepoints (indicated by *) are illustrated at the phylum, genus, and species level. Darker grey and lighter grey coloration indicates enrichment at the specified time point in control and in PAT specimens, respectively. FIGS. 44A-C show that PAT decreases the relative abundance of Bifidobacterium genus (A) and three detectable Bifidobacterium species (B. animalis, B. adolescentis and B. pseudolongum) (B) and unnamed Bifidobacterium species (C) in both females and males. The V4 region of the 16S rRNA gene was sequenced for 205 control and PAT samples from both male and female NOD mice. OTU picking and taxonomic assignment were performed using QIIME. Panel A. Relative abundance of Bifidobacterium genus. Each dot represents a sample, and the horizontal line represents the mean. Panel B. Relative abundance of 3 detectable Bifidobacterium species. The detectable named Bifidobacterium species were B. adolescentis, B. animalis, and B. pseudolongum. In both males and females, Bifidobacterium pseudolongum appears to be the dominant bifidobacterium species. In controls (black bars), Bifidobacteria especially B. pseudolongum are more highly abundant in males. In females, Bifidobacteria are very infrequently seen in the ileum while in males they are abundant (light grey bar). In females, PAT decreases the abundance of the three identified species of Bifidobacterium but there is some recovery by 13 weeks. STAT has increased abundance of all 3 of these species in females. In males, as with females, PAT decreases the abundance of these three species, B. animalis, B. adolescentis and B. pseudolongum, all of which have previously identified in human intestinal samples. However, in males unlike in the females there is very little or no recovery in the identified ileal Bifidobacterium. STAT appears similar to controls in fecal samples, with slightly reduced bifidobacteria species in the ileum. Panel C. Relative abundance of unnamed Bifidobacterium species. Bar height represents the mean and error bars depict the SEM. The unnamed Bifidobacterium species is present at a higher relative abundance in male controls, up to nearly 6% compared with the named species, which have a relative abundance below 1%. The unnamed Bifidobacterium species is a species or group of species that was only able to be identified down to the species level based on the sequencing data and current reference databases and can represent any species within this genus. Control male NOD mice have higher relative abundance of Bifidobacterium s than females in fecal samples (black bars) and ileal samples (light grey bars). Control male samples have higher relative abundance of Bifidobacterium s than PAT-treated mice, and the same trend is seen in females, although PAT females recover some Bifidobacterium s_{—} by the 13 weeks (last lighter grey bar).

FIG. 47 shows taxa with significantly different abundances in control and PAT male NOD mice across timepoints and locations. Area under the curve analysis was used to determine significantly different taxa, with FDR correction for multiple comparisons. Taxa with at least two significant timepoints (indicated by *) are illustrated at the phylum, genus, and species level. Dark grey and light grey coloration indicates enrichment at the specified time point in control and in PAT specimens, respectively.

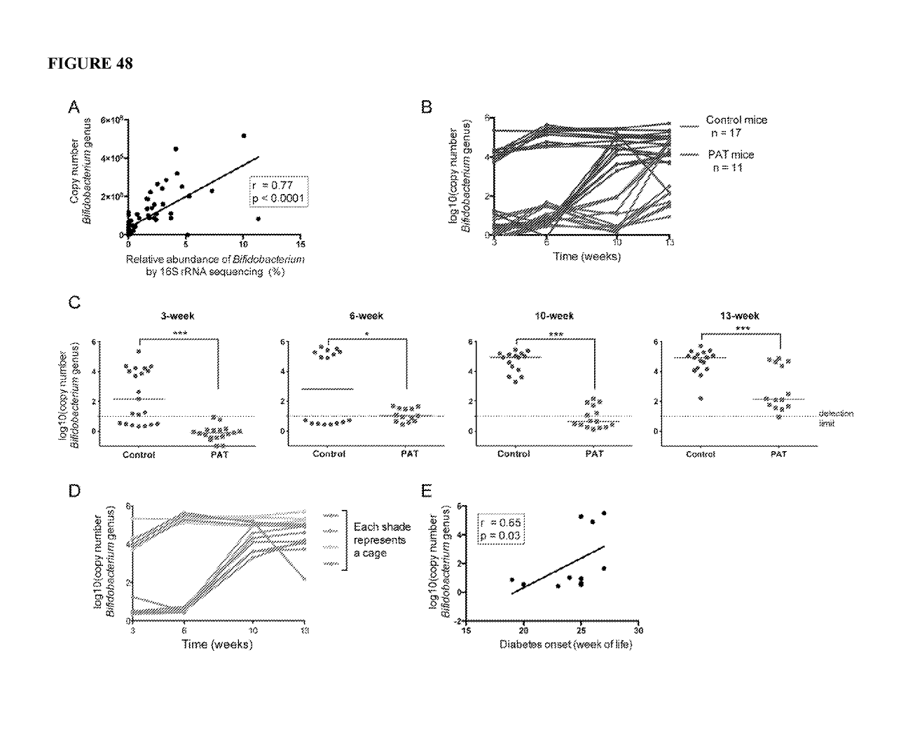

FIGS. 48A-E show the quantitation of Bifidobacterium by qPCR using Bifidobacterium genus-specific primers (F 5′ CTCCTGGAAACGGGTGG 3′ (SEQ ID NO: 56); R 5′ GGTGTTCTTCCCGATATCTACA 3′ (SEQ ID NO: 57)). Testing was performed on control and PAT male fecal samples at 4 timepoints: 3-, 6-, 10-, and 13-weeks of life. Panel A depicts the correlation of Bifidobacterium genus quantitation by qPCR and relative abundance by 16S rRNA sequencing. Spearman correlation was performed to determine statistical significance. Only samples with defined counts of Bifidobacterium by sequencing were used for this analysis. Panel B shows patterns of genus Bifidobacterium colonization over time. Panel C shows the quantitation of genus Bifidobacterium at 3-, 6-, 10-, and 13-weeks of life. The unpaired t-test was used to determine statistical significance * p<0.05, *** p<0.001. The median for each group was calculated and displayed as a line. Panel D shows Bifidobacterium levels in control mice by cage. Panel E shows that age of diabetes onset negatively correlates with Bifidobacterium copy number at 6-weeks of age. Spearman correlation was performed to determine statistical significance. Only samples from mice that develop diabetes were included in this analysis.

TABLE 6
Prebiotics to promote growth of Bifidobacteria.

| | | Refer- |
|---|---|---|
| Compound Name | Sources | ence(s) |
| Fructooligosaccha- | Raftilose p95 short-chain FOS (DP ≦9), | [1,2,3, |
| rides (FOS) | BENEO-orafti, Germany | 4,5,6] |
| | Actilight (short-chain FOS (DP ≦4)), | [7] |
| | Beghin Meiji Industries, France | |
| Inulin (+FOS) | Raftiline HP, BENEO-orafti | [1,2,6, |
| | Frutafit IQ, Sensus, Netherlands | 8,9,10, |
| | Sigma-Aldrich, St. Louis, MO | 11,12] |
| | Fibruline Instant, Cosucra, Belgium | |
| Galactooligosaccha- | Purimune, GTC Nutrition, Golden, CO | [2,13, |
| ride (GOS) | Oligomate55, Yakult Institute, Japan. | 14] |
| Human milk | No known commercial source | [2] |
| oligosaccharide | | |
| (HMO) | | |
| Lacto-N-neotetraose | Glycom, Denmark | [15] |
| D-Tagatose | Sigma-Aldrich | [16] |
| Xylo-oligosaccha- | Sigma-Aldrich | [14,17, |
| rides (XOS) | Shandong Longlive Bio-Technology, China | 18,19] |
| (+Xylose) | Suntory, Japan | |
| | Lenzing AG, Lenzing, Austria | |
| | Opti'flor; DF3 SAS, France | |
| Arabinoxylan- | Sigma-Aldrich | [17,20, |
| oligosaccharides | Fugeia, Belgium | 21] |
| (AXOS) | | |

### TABLE 6 REFERENCE CITATIONS

1. Scott K P, Martin J C, Duncan S H, Flint H J. Prebiotic stimulation of human colonic butyrate-producing bacteria and bifidobacteria, in vitro. FEMS microbiology ecology 2013. [Epub ahead of print: doi: 10.1111/1574-6941.12186]
2. Kim J H, An H J, Garrido D, German J B, Lebrilla C B, Mills D A. Proteomic analysis of Bifidobacterium longum subsp. infantis reveals the metabolic insight on consumption of prebiotics and host glycans. PloS one 2013; 8:e57535.
3. Wang X, Gibson G R. Effects of the in vitro fermentation of oligofructose and inulin by bacteria growing in the human large intestine. The Journal of applied bacteriology 1993; 75:373-80.
4. Delzenne N M, Cani P D, Neyrinck A M. Modulation of glucagon-like peptide 1 and energy metabolism by inulin and oligofructose: experimental data. J Nutrition 2007; 137:2547S-51S.
5. Likotrafiti E, Tuohy K M, Gibson G R, Rastall R A. Development of antimicrobial synbiotics using potentially-probiotic faecal isolates of Lactobacillus fermentum and Bifidobacterium longum. Anaerobe 2013; 20:5-13.
6. Gibson G R, Beatty E R, Wang X, Cummings J H. Selective stimulation of bifidobacteria in the human colon by oligofructose and inulin. Gastroenterology 1995; 108:975-82.
7. Bouhnik Y, Vahedi K, Achour L, et al. Short-chain fructo-oligosaccharide administration dose-dependently increases fecal bifidobacteria in healthy humans. The Journal of nutrition 1999; 129:113-6.
8. Kolida S, Meyer D, Gibson G R. A double-blind placebo-controlled study to establish the bifidogenic dose of inulin in healthy humans. European journal of clinical nutrition 2007; 61:1189-95.
9. Kuo S M, Merhige P M, Hagey L R. The effect of dietary prebiotics and probiotics on body weight, large intestine indices, and fecal bile acid profile in wild type and IL10−/− mice. PloS one 2013; 8:e60270.
10. Strompfova V, Laukova A, Cilik D. Synbiotic administration of canine-derived strain Lactobacillus fermentum CCM 7421 and inulin to healthy dogs. Canadian journal of microbiology 2013; 59:347-52.
11. Bouhnik Y, Raskine L, Champion K, et al. Prolonged administration of low-dose inulin stimulates the growth of bifidobacteria in humans. Nutr Res 2007; 27:187-93.
12. Kruse H P, Kleessen B, Blaut M. Effects of inulin on faecal bifidobacteria in human subjects. British J Nutrition 1999; 82:375-82.
13. Bouhnik Y, Flourie B, D'Agay-Abensour L, et al. Administration of transgalacto-oligosaccharides increases fecal bifidobacteria and modifies colonic fermentation metabolism in healthy humans. J Nutrition 1997; 127:444-8.
14. Santos A, San Mauro M, Diaz D M. Prebiotics and their long-term influence on the microbial populations of the mouse bowel. Food Microbiology 2006; 23:498-503.
15. Marcobal A, Barboza M, Sonnenburg E D, et al. Bacteroides in the infant gut consume milk oligosaccharides via mucus-utilization pathways. Cell Host & Microbe 2011; 10:507-14.
16. Zinedine A, Faid M. Isolation and Characterization of strains of Bifidobacteria with Probiotic Properties In vitro. World Journal of Dairy & Food Sciences 2007; 2:28-34.
17. Riviere A, Moens F, Selak M, Maes D, Weckx S, De Vuyst L. The ability of bifidobacteria to degrade arabinoxylan-oligosaccharide constituents and derived oligosaccharides is strain-dependent. Applied Environmental Microbiol 2014; 2014; 80:204-17.
18. Makelainen H, Forssten S, Saarinen M, Stowell J, Rautonen N, Ouwehand A C. Xylo-oligosaccharides enhance the growth of bifidobacteria and Bifidobacterium lactis in a simulated colon model. Beneficial Microbes 2010; 1:81-91.
19. Lecerf J M, Depeint F, Clerc E, et al. Xylo-oligosaccharide (XOS) in combination with inulin modulates both the intestinal environment and immune status in healthy subjects, while XOS alone only shows prebiotic properties. British J Nutrition 2012; 108:1847-58.
20. Walton G E, Lu C, Trogh I, Arnaut F, Gibson G R. A randomised, double-blind, placebo controlled cross-over study to determine the gastrointestinal effects of consumption of arabinoxylan-oligosaccharides enriched bread in healthy volunteers. Nutr J 2012; 11:36.
21. Pastell H, Westermann P, Meyer A S, Tuomainen P, Tenkanen M. In vitro fermentation of arabinoxylan-derived carbohydrates by bifidobacteria and mixed fecal microbiota. Journal of Agricultural and Food Chemistry 2009; 57:8598-606.
22. Biasucci G, Benenati B, Morelli L, Bessi E, Boehm G. Cesarean delivery may affect the early biodiversity of intestinal bacteria. J Nutr 2008; 138: 1796S-1800S. PubMed: 18716189.
23. Huurre A, Kalliomäki M, Rautava S, Rinne M, Salminen S et al. Mode of delivery—effects on gut microbiota and humoral immunity. Neonatology 2008; 93: 236-240. doi:10.1159/000111102. PubMed: 18025796

The present data show that at multiple points over time and in multiple types of samples (fecal, cecal, and ileal), high relative abundance of Bifidobacterium sp. is associated with diabetes protection and that Akkermansia mucinophilia is associated with diabetes progression. This specificity, consistency, and time-series type of study is novel for studying the microbiota in relation to type 1 diabetes. Importantly, unlike the prior studies (Marietta et al., PloS One 2013, 8: e78687; Murri et al., BMC Medicine 2013, 11:46; de Goffau et al., Diabetes 2013, 62:1238-44; Hara et al., J. Immunol., 2012, 189:3805-14; Uibo et al., Cellular & Molecular Immunology 2011, 8:150-6), the present example demonstrates that higher levels of Bifidobacterium sp. are present early in life, by many weeks predating diabetes onset, thus implying protective role of Bifidobacterium sp. against diabetes.

The protective role of Bifidobacterium sp. against type 1 diabetes demonstrated herein is also supported by the biological differences between male and female NOD mice. It has been long-recognized in NOD mouse colonies around the world that male NOD mice are relatively protected from diabetes compared to females. As demonstrated herein, Bifidobacterium sp are relatively (and significantly) enriched in male NOD mice, which are relatively protected from diabetes compared to females. Similarly, Bifidobacterium sp. are more prevalent in control males than in PAT-treated males, who have a higher rate of type 1 diabetes. Finally, the prevalence of Bifidobacterium sp. is not different in STAT-antibiotic-treated mice, who are no different from controls in type 1 diabetes expression.

The present findings on the protective role of Bifidobacterium sp. against type 1 diabetes are consistent with data that Bifidobacterium are less abundant in children from highly developed countries such as Finland with a very high incidence of type 1 diabetes than in children from less developed countries such as Malawi, which have a much lower incidence of type 1 diabetes (Grzeskowiak et al., J. Ped. Gastroenterol. and Nutrition 2012, 54:812-6; Karvonen et al., Diabetes Care 2000, 23:1516-26). The present findings are also consistent with the data that a higher rate of type 1 diabetes is associated with C-sections and formula feeding, both of which have been independently associated with lower amounts of Bifidobacterium (Yatsunenko et al., Nature 2012, 486:222-11; Cardwell et al., Diabetes Care 2012, 35:2215-25; Penders et al., Pediatrics 2006, 118:511-21; Gronlund et al., J. Ped. Gastroenterol. and Nutrition 1999, 28:19-25).

### Example 6

### Immunomodulatory Effects of Oropharyngeal and Vaginal Microbiota

The oropharyngeal compartment is colonized with hundreds of bacterial species. The most prevalent phyla in the oropharynx are Firmicutes, Proteobacteria and Bacteroidetes with an inverse relationship between Firmicutes and Proteobacteria (Lemon, K. et al. 2009; mBio 1). These communities may regulate the pathogenicity of other microbes present in the oropharyngeal niche. One such organism is the fungal species Candida albicans. An immunocompromised host or a host with antibiotic-mediated perturbation of the oropharyngeal micro biota can sustain shifts of C. albicans from commensal to pathogen (Pirofski, L. et al. J. Ex. Med. 2009, 206:269-273) Th17 populations are considered crucial in the immune defense against oral candidiasis and can induce long-term adaptive immune responses (Conti, H. et al. J. Ex. Med. 2009, 206:299-311, Hernandez-Santos, N. et al. 2012. doi:10.1038/mi.2012.128). Th17 populations may be able to be modulated by the oropharyngeal commensal microbiota through shifts in Firmicutes and Proteobacteria.

The vaginal microbiota is composed of numerous bacterial species (Lamont, R. et al. 2011. BJOG 118, 533-49). In most healthy women, the vaginal microbiota is dominated by Lactobacillus species (Ma, B., et al. 2011 Ann. Rev. Micro. 66, 371-89). Together the vaginal microbiota play a key role in antimicrobial defense against many vaginal pathogens. One such pathogen may be the fungal species Candida albicans. CD4+ lymphocytes have been identified during Candida infection, and a specific CD4 subset, Th17, is considered protective in vaginal candidiasis (Ildgruben et al. (2003) Obstet Gynecol 102: 571-582; Pietrella, D. et al. (2010) PloS one 6, e22770). The vaginal microbiota is dynamic (Gajer, P. et al., Science Translational Research. 4, 132ra52 (2012)) and may influence CD4+ immune responses through shifts in bacterial communities. Shifts in vaginal microbiota may influence Th17 differentiation and promote protection against Candida infection.

### Example 7

### Studies in Mice to Determine Whether Augmenting Bifidobacterium sp with Prebiotic Sugars can Protect from the Development of Diabetes

The goal of the study is to determine whether augmentation of Bifidobacterium sp. by feeding mice prebiotic sugars that differentially promote Bifidobacterium sp. prevents Bifidobacterium sp. loss resulting from antibiotic treatment and can protect from the development of diabetes. The prebiotic sugars listed in Table 5 are given alone or in combination to NOD mice in the drinking water and/or feed to promote the growth of Bifidobacterium. The initial studies are used to determine the optimal concentration and combination of prebiotic sugars to promote the growth of Bifidobacterium. DNA is extracted from fecal samples collected before and after administration of Bifidobacterium and quantitative PCR (qPCR) is performed to determine the amount of Bifidobacterium present before and after treatment using previously described 16S rRNA-specific primers (Matsuki et al., Applied and Environmental Microbiology, 1999; 65:4506-12; Matsuki et al., Applied and Environmental Microbiology, 2004; 70:7220-8; Matsuki et al., Applied and Environmental Microbiology, 2004; 70:167-73). Using the determined optimal growth promoting prebiotic combination and concentration, it is then tested whether augmention of Bifidobacterium in antibiotic-treated mice can protect against the development of diabetes. To do this, 4 groups of mice are used: (1) control mice that receive no prebiotics, (2) control mice that receive prebiotics, (3) PAT-treated mice that receive no prebiotics, and (4) PAT-treated mice that receive prebiotics. It is then determined (i) whether the PAT-treated mice which receive the prebiotics are relatively protected from diabetes as compared to the PAT-treated mice that do not receive the prebiotics and (ii) whether the diabetes incidence of the PAT-treated mice which receive the prebiotics is similar to that seen in the controls.

### Example 8

### Studies in Mice to Determine Whether Providing Bifidobacterium sp Before, During, or after Antibiotic Exposure can Abrogate the Increased Diabetes Risk

The goal of the study is to determine whether adding Bifidobacterium sp back to mice before, during, or after antibiotic exposure can abrogate the increased diabetes risk. To perform these studies, Bifidobacterium sp. are isolated from fecal and cecal samples previously collected from NOD mice using anaerobic culturing techniques (see, e.g., Manual of Clinical Microbiology, American Society for Microbiology). These Bifidobacterium sp. are administered as single species inoculums or as combinations to NOD mice to determine whether they can protect from the development of type 1 diabetes. The bacteria are diluted to 10⁷-10⁸ cells in 0.1-0.5 ml of sterile anaerobic media and are delivered via oral gavage. To determine the role of the administered bacteria in diabetes protection, 6 groups of mice are used: (1) control mice (no antibiotics, no probiotics), (2) control mice that received probiotic at weaning (3 weeks of age), (3) control mice that received probiotic at 6-7 weeks of age, (4) PAT-treated mice (antibiotics, no probiotics), (5) PAT-treated mice that received probiotic at weaning, and (6) PAT-treated mice that received probiotic at 6-7 weeks of age.

### Example 9

### Studies of Bifidobacterium Species in Children

The study is conducted in a cohort of children from birth through the first year of life. The acquisition of Bifidobacterium is followed in relation to age and antibiotic treatment. From these studies it is determined which Bifidobacterium species are most normally acquired through vaginal, but not Caesarean birth, and whose colonization is enhanced through breastfeeding but not formula feeding. In addition, the study seeks to determine which Bifidobacterium species are preferentially lost after antibiotic treatment and/or transition to formula feeding. This will lead to particular candidate species.

Fecal samples as well as epidemiological data including delivery mode, antibiotic use, and breastfeeding versus formula feeding duration is collected from a cohort of children. DNA is extracted from fecal samples as described for mouse fecal samples in Example 5, above, using the PowerLyzer™ PowerSoil® DNA Isolation Kit (MoBio, Carlsbad Calif.). The concentration of Bifidobacterium sp. is determined using previously described 16S rRNA-specific primers (Matsuki et al., Applied and Environmental Microbiology, 1999; 65:4506-12; Matsuki et al., Applied and Environmental Microbiology, 2004; 70:7220-8; Matsuki et al., Applied and Environmental Microbiology, 2004; 70:167-73).

### Example 10

### Studies of Bifidobacterium Species in Young Children at High Risk of Developing Type 1 Diabetes

Using fecal samples collected during the Environmental Determinants of Diabetes in the Young (TEDDY) study of young children at high risk of developing type 1 diabetes (Ann. NY Acad. Sci., 2008, 1150:1-13) and healthy controls, identify Bifidobacterium species in children who will ultimately progress to type 1 diabetes. The concentration of Bifidobacterium sp. is determined using previously described 16S rRNA-specific primers (Matsuki et al., Applied and Environmental Microbiology, 1999; 65:4506-12; Matsuki et al., Applied and Environmental Microbiology, 2004; 70:7220-8; Matsuki et al., Applied and Environmental Microbiology, 2004; 70:167-73).

### REFERENCES

Anderson M S, Bluestone J A. The NOD mouse: a model of immune dysregulation Annu Rev Immunol. 23:447-85 (2005).
Atkinson M A, Leiter E H. The NOD mouse model of type 1 diabetes: as good as it gets? Nat Med. 5, 601-4 (1999).
Bach, J.-F. The effect of infections on susceptibility to autoimmune and allergic diseases. The New England journal of medicine. 347, 911-20 (2002).
Barman M, Unold D, Shifley K, Amir E, Hung K, Bos N, et al. Enteric salmonellosis disrupts the microbial ecology of the murine gastrointestinal tract. Infection and immunity. 76, 907-15 (2008).
Braumüller, H. et al. T-helper-1-cell cytokines drive cancer into senescence. Nature 494, 361-5 (2013).
Broide, D. Immunomodulation of allergic disease Annual review of medicine 60, 279-91 (2008).
Burisch, J. & Munkholm, P. Inflammatory bowel disease epidemiology. Current opinion in gastroenterology 29:000-000 (2013).
Cao X Y et al. Tilmicosin and tylosin have anti-inflammatory properties via modulation of COX-2 and iNOS gene expression and production of cytokines in LPS induced macrophages and monocytes. International Journal of Antimicrobial Agents. 27:431-438 (2006)
Cho, I. et al. Antibiotics in early life alter the murine colonic microbiome and adiposity. Nature 488, 621-6 (2012).
Dabelea, D. The accelerating epidemic of childhood diabetes. Lancet 373, 1999-2000 (2009).
De Monte, L. et al. Intratumor T helper type 2 cell infiltrate correlates with cancer-associated fibroblast thymic stromal lymphopoietin production and reduced survival in pancreatic cancer. The Journal of experimental medicine 208, 469-78 (2011).
Filippi, M. Multiple sclerosis in 2010: Advances in monitoring and treatment of multiple sclerosis. Nature reviews. Neurology 7, 74-5 (2011).
Forestier C, Takaki T, Molano A, Im J S, Baine I, Jerud E S, et al. Improved outcomes in NOD mice treated with a novel Th2 cytokine-biasing NKT cell activator. Journal of immunology, 178(3):1415-25 (2007).
Gajer P. et al. Temporal dynamics of the human vaginal microbiota. Science Translational Research. 4, 132ra52 (2012).
Green A, Patterson C C. Trends in the incidence of childhood-onset diabetes in Europe 1989-1998. Diabetologia, 44 Suppl 3:B3-8. (2001).
Grivennikov, S. et al. Adenoma-linked barrier defects and microbial products drive IL-23/I L-17-mediated tumour growth. Nature 491, 254-8 (2012).
Hobbs, K. & Cohen, M. Rheumatoid arthritis disease measurement: a new old idea. Rheumatology, 51 Suppl 6, vi21-7 (2012).
Holt, P. & Jones, C. The development of the immune system during pregnancy and early life. Allergy 55, 688-97 (2000).
Hooper, L., Littman, D. & Macpherson, A. Interactions between the microbiota and the immune system. Science (New York, N.Y.) 336, 1268-73 (2012).
Ivanov, I I, Atarashi K, Manel N, et al. Induction of intestinal Th17 cells by segmented filamentous bacteria. Cell 139:485-98 (2009).
Jonsson, H. Segmented filamentous bacteria in human ileostomy samples after high-fiber intake. FEMS microbiology letters 342, 24-9 (2013).
Kanoh, S. & Rubin, B. Mechanisms of action and clinical application of macrolides as immunomodulatory medications. Clinical microbiology reviews 23, 590-615 (2010).
Kaser, A., Zeissig, S. & Blumberg, R. Inflammatory bowel disease Annual review of immunology 28, 573-621 (2009).
Klaasen, H. L., Koopman, J. P., Van den Brink, M. E., Bakker, M. H., and Beynen, A. C. Influence of a natural-ingredient diet containing Phaseolus vulgaris on the colonization by segmented, filamentous bacteria of the small bowel of mice. Int J Vitam Nutr Res 62, 334-341. (1992)
Koga, C., Kabashima, K., Shiraishi, N., Kobayashi, M. & Tokura, Y. Possible pathogenic role of Th17 cells for atopic dermatitis. The Journal of investigative dermatology 128, 2625-30 (2008).
Leiter E H. The NOD mouse: a model for insulin-dependent diabetes mellitus. Current Protocols in Immunology, Chapter 15:Unit 15 9 (2001).
Lloyd, C. & Hawrylowicz, C. Regulatory T cells in asthma. Immunity 31, 438-49 (2009).
Lloyd, C. & Hessel, E. Functions of T cells in asthma: more than just T(H)2 cells. Nature reviews. Immunology 10, 838-48 (2010).
Ludvigsson, J. F. et al. Increasing Incidence of Celiac Disease in a North American Population. The American Journal of Gastroenterology 108, (2013).
Mathis, D. & Benoist, C. The influence of the microbiota on type-1 diabetes: on the threshold of a leap forward in our understanding. Immunological reviews 245, 239-49 (2011).
Markle, J. et al. Sex differences in the gut microbiome drive hormone-dependent regulation of autoimmunity. Science (New York, N.Y.) 339, 1084-8 (2013).
Nguyen, T.-H. T. & Casale, T. Immune modulation for treatment of allergic disease. Immunological reviews 242, 258-71 (2011)
Nurmohamed, M. & Dijkmans, B. Are biologics more effective than classical disease-modifying antirheumatic drugs? Arthritis research & therapy 10, 118 (2007).
Patterson, C., Dahlquist, G., Gyürüs, E., Green, A. & Soltész, G. Incidence trends for childhood type 1 diabetes in Europe during 1989-2003 and predicted new cases 2005-20: a multicentre prospective registration study. Lancet 373, 2027-33 (2009).
Reigstad C S, Lunden G O, Felin J, Backhed F. Regulation of serum amyloid A3 (SAA3) in mouse colonic epithelium and adipose tissue by the intestinal microbiota. PloS one, 4(6):e5842 (2009).
Round, J. & Mazmanian, S. Inducible Foxp3+ regulatory T-cell development by a commensal bacterium of the intestinal microbiota. Proceedings of the National Academy of Sciences of the United States of America 107, 12204-9 (2010).
Savage, D. Microbial ecology of the gastrointestinal tract Annual review of microbiology 31, 107-33 (1976)
Sczesnak, A. et al. The genome of th17 cell-inducing segmented filamentous bacteria reveals extensive auxotrophy and adaptations to the intestinal environment. Cell host & microbe 10, 260-72 (2011).
Simpson, J., Powell, H., Boyle, M., Scott, R. & Gibson, P. Clarithromycin targets neutrophilic airway inflammation in refractory asthma. American journal of respiratory and critical care medicine 177, 148-55 (2008).
Slack, E. et al Innate and adaptive immunity cooperate flexibly to maintain host-microbiota mutualism. Science (New York, N.Y.) 325, 617-20 (2009).
Smyth, G. K. Linear models and empirical Bayes methods for assessing differential expression in microarray experiments. Statistical Applications in Genetics and Molecular Biology 3, No. 1, Article 3. (2004).
Snyder C M, Rice A L, Estrella N L, Held A, Kandarian S C, Naya F J. MEF2A regulates the Gt12-Dio3 microRNA mega-cluster to modulate WNT signaling in skeletal muscle regeneration. Development. 140(1):31-42 (2013).
Son D S, Roby K F, Terranova P F. Tumor necrosis factor-alpha induces serum amyloid A3 in mouse granulosa cells. Endocrinology, 145(5):2245-52 (2004).
Spandidos A, Wang X, Wang H, Seed B. PrimerBank: a resource of human and mouse PCR primer pairs for gene expression detection and quantification. Nucleic acids research, 38 D792-9. (2010).
Staeva, T., Chatenoud, L., Insel, R. & Atkinson, M. Recent lessons learned from prevention and recent-onset type 1 diabetes immunotherapy trials. Diabetes 62, 9-17 (2012).
Tullman, M. Overview of the epidemiology, diagnosis, and disease Progression Associated With multiple Sclerosis. Am J Manag Care. 2013; 19:S21-S27 (2013)
Vehik, K. et al. Increasing incidence of type 1 diabetes in 0- to 17-year-old Colorado youth. Diabetes care 30, 503-9 (2007).
Vijay-Kumar, M. et al. Metabolic syndrome and altered gut microbiota in mice lacking Toll-like receptor 5. Science (New York, N.Y.) 328, 228-31 (2010).
Conti, H. et al. Th17 cells and IL-17 receptor signaling are essential for mucosal host defense against oral candidiasis. The Journal of experimental medicine 206, 299-311 (2009).
Lemon, K. et al. Comparative analyses of the bacterial microbiota of the human nostril and oropharynx. mBio 1, (2009).
Hernández-Santos, N. et al. Th17 cells confer long-term adaptive immunity to oral mucosal Candida albicans infections. Mucosal immunology (2012). doi: 10 0.1038/mi.20120.128
Pirofski, L. & Casadevall, A. Rethinking T cell immunity in oropharyngeal candidiasis. The Journal of experimental medicine 206, 269-73 (2009).
Gajer, P. et al. Temporal Dynamics of the Human Vaginal Microbiota. Science Translational Medicine 4, (2012).
Ildgruben et al. (2003) Influence of hormonal contraceptives on the immune cells and thickness of human vaginal epithelium. Obstet Gynecol 102: 571-582.).
Lamont, R. et al. The vaginal microbiome: new information about genital tract flora using molecular based techniques. BJOG: an international journal of obstetrics and gynaecology 118, 533-49 (2011).
Ma, B., Forney, L. & Ravel, J. Vaginal microbiome: rethinking health and disease Annual review of microbiology 66, 371-89 (2011).)
Pietrella, D. et al. Th17 cells and IL-17 in protective immunity to vaginal candidiasis. PloS one 6, e22770 (2010))

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A method for decreasing Th17 and/or Treg and/or Th2 cell differentiation in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

2. A method for decreasing Th17 and/or Treg and/or Th2 cell differentiation in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition results in a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

3. A method for treating a disease treatable by decreasing Th17 and/or Treg and/or Th2 cell differentiation in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

4. A method for treating a disease treatable by decreasing Th17 and/or Treg and/or Th2 cell differentiation in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition results in a decrease in Th17 and/or Treg and/or Th2 cell differentiation in the intestine and/or tonsils and/or waldeyer's ring and/or vagina of the mammal, which decrease is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

5. The method of claim 1 or 3, wherein said bacterial species is from the order Bacteroidales.

6. The method of claim 1 or 3, wherein said bacterial species is from a family selected from the group consisting of Lachnospiraceae, Clostridiaceae, Bacteroidaceae, Bifidobacteriaceae, and Enterobacteriaceae.

7. The method of claim 1 or 3, wherein said bacterial species is from a genus selected from the group consisting of Akkermansia, Odoribacter, Enterococcus, Candidatus Arthromitus (segmented filamentous bacteria), Bacteroides, Blautia, Bifidobacterium, and Proteus.

8. The method of claim 1 or 3, wherein said bacterial species is selected from the group consisting of Bacteroides uniformis, Blautia producta, Bifidobacterium pseudolongum, and Akkermansia muciniphila.

9. The method of claim 2 or 4, wherein said bacterial strain is from the order Bacteroidales.

10. The method of claim 2 or 4, wherein said bacterial strain is from a family selected from the group consisting of Lachnospiraceae, Clostridiaceae, Bacteroidaceae, Bifidobacteriaceae, and Enterobacteriaceae.

11. The method of claim 2 or 4, wherein said bacterial strain is from a genus selected from the group consisting of Akkermansia, Odoribacter, Enterococcus, Candidatus Arthromitus (segmented filamentous bacteria), Bacteroides, Blautia, Bifidobacterium, and Proteus.

12. The method of claim 2 or 4, wherein said bacterial strain is from the species selected from the group consisting of Bacteroides uniformis, Blautia producta, Bifidobacterium pseudolongum, and Akkermansia muciniphila.

13. A method for decreasing the expression of at least one serum amyloid A (SAA) gene in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes a decrease in the expression of said SAA gene(s) in the epithelium and/or lamina propria of the intestines and/or tonsils and/or waldeyer's ring and/or vagina of a mammal.

14. A method for decreasing the expression of at least one serum amyloid A (SAA) gene in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition causes a decrease in the expression of said SAA gene(s) in the epithelium and/or lamina propria of the intestines and/or tonsils and/or waldeyer's ring and/or vagina of a mammal.

15. A method for treating a disease treatable by decreasing the expression of at least one serum amyloid A (SAA) gene in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes a decrease in the expression of said SAA gene(s) in the epithelium and/or lamina propria of the intestines and/or tonsils and/or waldeyer's ring and/or vagina of a mammal.

16. A method for treating a disease treatable by decreasing the expression of at least one serum amyloid A (SAA) gene in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition causes a decrease in the expression of said SAA gene(s) in the epithelium and/or lamina propria of the intestines and/or tonsils and/or waldeyer's ring and/or vagina of a mammal.

17. The method of claim 13 or 15, wherein said bacterial species is from a genus selected from the group consisting of Akkermansia, Sutterella, and Blautia.

18. The method of claim 13 or 15, wherein said bacterial species is Akkermansia muciniphila or Blautia producta.

19. The method of claim 14 or 16, wherein said bacterial strain is from a genus selected from the group consisting of Akkermansia, Sutterella, and Blautia.

20. The method of claim 14 or 16, wherein said bacterial strain is from the species Akkermansia muciniphila or Blautia producta.

21. The method of any one of claims 3, 4, 15, and 16, wherein the disease is selected from the group consisting of multiple sclerosis (MS), rheumatoid arthritis (RA), asthma, inflammatory bowel diseases, atopic diseases, Systemic Lupus Erythematosis (SLE), amyotrophic lateral sclerosis (ALS), sarcoidosis, scleroderma, thyroiditis, idiopathic pulmonary fibrosis, psoriasis, and graft versus host disease.

22. The method of claim 21, wherein the inflammatory bowel disease is selected from the group consisting of ulcerative colitis, Crohn's disease, and celiac disease.

23. The method of claim 21, wherein the atopic disease is selected from the group consisting of atopic dermatitis, allergic rhinitis, conjunctivitis, and other allergic diseases.

24. A method for increasing Th17 and/or Treg and/or Th2 cell differentiation in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

25. A method for increasing Th17 and/or Treg and/or Th2 cell differentiation in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition results in an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

26. A method for treating a disease treatable by increasing Th17 and/or Treg and/or Th2 cell differentiation in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

27. A method for treating a disease treatable by increasing Th17 and/or Treg and/or Th2 cell differentiation in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition results in an increase in Th17 and/or Treg and/or Th2 cell differentiation in the intestine and/or tonsils and/or waldeyer's ring and/or vagina of the mammal, which increase is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

28. The method of claim 24 or 26, wherein said bacterial species is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae.

29. The method of claim 24 or 26, wherein said bacterial species is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella.

30. The method of claim 24 or 26, wherein said bacterial species is selected from the group consisting of Bacteroides uniformis, Blautia producta, Bifidobacterium pseudolongum, and Lactobacillus reuteri.

31. A method for increasing the expression of at least one serum amyloid A (SAA) gene in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes an increase in the expression of said SAA gene(s) in the epithelium and/or lamina propria of the intestines and/or tonsils and/or waldeyer's ring and/or vagina of a mammal.

32. A method for increasing the expression of at least one serum amyloid A (SAA) gene in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition causes an increase in the expression of said SAA gene(s) in the epithelium and/or lamina propria of the intestines and/or tonsils and/or waldeyer's ring and/or vagina of a mammal.

33. A method for treating a disease treatable by increasing the expression of at least one serum amyloid A (SAA) gene in the intestine and/or tonsils and/or waldeyer's ring and/or vagina of a mammal in need thereof comprising enhancing growth or activity of at least one bacterial species in the intestinal and/or oropharyngeal and/or vaginal microbiota of said mammal, wherein said bacterial species causes an increase in the expression of said SAA gene(s) in the epithelium and/or lamina propria of the intestines and/or tonsils and/or waldeyer's ring and/or vagina of a mammal.

34. A method for treating a disease treatable by increasing the expression of at least one serum amyloid A (SAA) gene in the intestine and/or tonsils and/or waldeyer's ring and/or vagina of a mammal in need thereof comprising administering to said mammal a therapeutically effective amount of a probiotic composition comprising at least one bacterial strain, or a combination of several strains, wherein the administration of the probiotic composition causes an increase in the expression of said SAA gene(s) in the epithelium and/or lamina propria of the intestines and/or tonsils and/or waldeyer's ring and/or vagina of a mammal.

35. The method of claim 31 or 33, wherein said bacterial species is from the order RF39 (class Mollicutes).

36. The method of claim 31 or 33, wherein said bacterial species is from a genus selected from the group consisting of Oscillospira, Odoribacter, and Bifidobacterium.

37. The method of claim 32 or 34, wherein said bacterial strain is from the order RF39 (class Mollicutes).

38. The method of claim 32 or 34, wherein said bacterial strain is from a genus selected from the group consisting of Oscillospira, Odoribacter, and Bifidobacterium.

39. The method of any one of claims 26, 27, 33, and 34, wherein the disease is type 1 diabetes (T1D).

40. The method of any one of claims 26, 27, 33, and 34, wherein the disease is selected from the group consisting of Crohn's disease, celiac disease, autism, oral candidiasis, and vaginal candidiasis.

41. The method of any one of claims 1, 3, 13, 15, 24, 26, 31, and 33, wherein enhancing growth or activity of at least one bacterial species in the microbiota comprises administering to said mammal a therapeutically effective amount of a probiotic composition comprising at least one bacterial strain of said bacterial species.

42. The method of any one of claims 2, 4, 14, 16, 25, 27, 32, 34, and 41, wherein the bacterial strain is a bacterial analogue or a conditionally lethal bacterial strain.

43. The method of any one of claims 2, 4, 14, 16, 25, 27, 32, 34, and 41, wherein the probiotic composition comprises live bacterial cells, killed bacterial cells, or bacterial extract.

44. The method of claim 43, wherein the probiotic composition further comprises a buffering agent.

45. The method of claim 44, wherein the buffering agent is selected from the group consisting of saline, sodium bicarbonate, milk, yogurt, infant formula, and other dairy products.

46. The method of claim 43, wherein the probiotic composition further comprises one or more prebiotic agents which enhance growth or activity of the bacterial strain.

47. The method of any one of claims 1, 3, 13, 15, 24, 26, 31, and 33, wherein enhancing growth or activity of at least one bacterial species in the microbiota comprises administering to said mammal a therapeutically effective amount of one or more prebiotic agents which enhance growth or activity of said bacterial species.

48. The method of claim 46 or 47, wherein the prebiotic agent is selected from the group consisting of fructooligosaccharides, galactooligosaccharides, N-acetylglucosamine, N-acetylgalactosamine, glucose, penta-saccharides, hexa-saccharides, amino acids, alcohols, and resistant starch (RS).

49. The method of claim 48, wherein the fructooligosaccharide is selected from the group consisting of oligofructose, inulin, and inulin-type fructans.

50. The method of any one of claims 2, 4, 14, 16, 25, 27, 32, 34, 41, and 47, wherein the probiotic composition and/or prebiotic is administered via a route selected from the group consisting of oral, rectal, fecal (by enema), and naso/oro-gastric gavage.

51. A method for decreasing Th17 and/or Treg and/or Th2 cell differentiation in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal.

52. A method for treating a disease treatable by decreasing Th17 and/or Treg and/or Th2 cell differentiation in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes an increase in Th17 and/or Treg and/or Th2 cell differentiation in the intestine and/or tonsils and/or waldeyer's ring and/or vagina of the mammal.

53. The method of claim 51 or 52, wherein said bacterial species is from a family selected from the group consisting of Clostridiaceae, Rikenellaceae, S24-7 (order Bacteroidales), Bacteroidaceae, Bifidobacteriaceae, Enterobacteriaceae, and Coriobacteriaceae.

54. The method of claim 51 or 52, wherein said bacterial species is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella.

55. The method of claim 51 or 52, wherein said bacterial species is selected from the group consisting of Bacteroides uniformis, Blautia producta, Bifidobacterium pseudolongum, and Lactobacillus reuteri.

56. The method of claim 51 or 52, wherein the increase in Th17 and/or Treg and/or Th2 cell differentiation is detectable as an increase in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

57. A method for decreasing the expression of at least one serum amyloid A (SAA) gene in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes an increase in the expression of said SAA gene(s) in the intestine and/or tonsils and/or waldeyer's ring and/or vagina of the mammal.

58. A method for treating a disease treatable by decreasing the expression of at least one serum amyloid A (SAA) gene in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes an increase in the expression of said SAA gene(s) in the intestine and/or tonsils and/or waldeyer's ring and/or vagina of the mammal.

59. The method of claim 57 or 58, wherein said bacterial species is from the order RF39 (class Mollicutes).

60. The method of claim 57 or 58, wherein said bacterial species is from a genus selected from the group consisting of Oscillospira, Odoribacter, and Bifidobacterium.

61. The method of claim 52 or 58, wherein the disease is selected from the group consisting of multiple sclerosis (MS), rheumatoid arthritis (RA), asthma, inflammatory bowel diseases, atopic diseases, Systemic Lupus Erythemtosis (SLE), amyotrophic lateral sclerosis (ALS), sarcoidosis, scleroderma, thyroiditis, idiopathic pulmonary fibrosis, psoriasis, and graft versus host disease.

62. The method of claim 61, wherein the inflammatory bowel disease is selected from the group consisting of ulcerative colitis, Crohn's disease, and celiac disease.

63. The method of claim 61, wherein the atopic disease is selected from the group consisting of atopic dermatitis, allergic rhinitis, conjunctivitis, and other allergic diseases.

64. A method for increasing Th17 and/or Treg and/or Th2 cell differentiation in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal.

65. A method for treating a disease treatable by increasing Th17 and/or Treg and/or Th2 cell differentiation in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes a decrease in Th17 and/or Treg and/or Th2 cell differentiation in the intestine and/or tonsils and/or waldeyer's ring and/or vagina of the mammal.

66. The method of claim 64 or 65, wherein said bacterial species is from the order Bacteroidales.

67. The method of claim 64 or 65, wherein said bacterial species is from a family selected from the group consisting of Clostridiaceae, Bacteroidaceae, Bifidobacteriaceae, Enterobacteriaceae, and Lachnospiraceae.

68. The method of claim 64 or 65, wherein said bacterial species is from a genus selected from the group consisting of Akkermansia, Odoribacter, Enterococcus, Candidatus Arthromitus (segmented filamentous bacteria), Bacteroides, Blautia, Bifidobacterium, and Proteus.

69. The method of claim 64 or 65, wherein said bacterial species is Akkermansia muciniphila.

70. The method of claim 64 or 65, wherein the decrease in Th17 and/or Treg and/or Th2 cell differentiation is detectable as a decrease in at least one parameter selected from the group consisting of Th17 and/or Treg and/or Th2 cell number, Th17 and/or Treg and/or Th2 cell activity, and expression of at least one Th17- and/or Treg- and/or Th2-specific cytokine, chemokine, or effector.

71. A method for increasing the expression of at least one serum amyloid A (SAA) gene in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes a decrease in the expression of said SAA gene(s) in the intestine and/or tonsils and/or waldeyer's ring and/or vagina of the mammal.

72. A method for treating a disease treatable by increasing the expression of at least one serum amyloid A (SAA) gene in the intestine and/or tonsils and/or waldeyer's ring and/or vagina of a mammal in need thereof comprising suppressing growth or activity of at least one bacterial species in the intestinal and/or oropharyngeal and/or vaginal microbiota of said mammal, wherein said bacterial species causes a decrease in the expression of said SAA gene(s) in the intestine and/or tonsils and/or waldeyer's ring and/or vagina of the mammal.

73. The method of claim 71 or 72, wherein said bacterial species is from a genus selected from the group consisting of Akkermansia, Sutterella, and Blautia.

74. The method of claim 71 or 72, wherein said bacterial species is Akkermansia muciniphila or Blautia producta.

75. The method of claim 65 or 72, wherein the disease is type 1 diabetes (T1D).

76. The method of claim 65 or 72, wherein the disease is selected from the group consisting of Crohn's disease, celiac disease, autism, oral candidiasis, and vaginal candidiasis.

77. The method of any one of claims 51, 52, 57, 58, 64, 65, 71, and 72, wherein suppressing growth or activity of at least one bacterial species in the microbiota comprises administering to said mammal an antibiotic.

78. The method of claim 77, wherein the antibiotic is administered in a therapeutic dose.

79. The method of claim 77, wherein the antibiotic is administered in a sub-therapeutic dose.

80. The method of claim 77, wherein the antibiotic is selected from the group consisting of beta-lactams, nitroimidazoles, macrolides, tetracyclines, glycopeptides, and fluoroquinolones.

81. The method of claim 80, wherein the beta-lactam is selected from the group consisting of Penicillin VK, Penicillin G, and Amoxicillin trihydrate.

82. The method of claim 80, wherein the macrolide is selected from the group consisting of Tylosin tartrate, Erythromycin, Azithromycin, and Clarithromycin.

83. The method of claim 80, wherein the glycopeptide is Vancomycin.

84. The method of claim 77, wherein the antibiotic is Penicillin VK or Penicillin G, which is administered at 1 mg/kg body weight per day for at least four weeks of life.

85. The method of claim 77, wherein the antibiotic is Amoxicillin trihydrate, which is administered at 25 mg/kg body weight per day for 1 to 3 treatments each lasting 3 to 5 days.

86. The method of claim 77, wherein the antibiotic is Tylosin tartrate, which is administered at 50 mg/kg body weight per day for 1 to 3 treatments each lasting 3 to 5 days.

87. The method of any one of claims 51, 52, 57, and 58, wherein suppressing growth or activity of at least one bacterial species in the microbiota results in a decrease in expression of one or more genes selected from the group consisting of Rab30, Areg, Flrt3, Arl5b, Maff, Dusp14, Zfand2a, Chka, Phlda1, Ereg, Tnfrsf12a, Il1rn, Il1rl1, Acs13, Slc2a1, S100a14, Klf4, and Gjb3, in the intestinal and/or oropharyngeal and/or vaginal epithelium and lamina propria of said mammal.

88. The method of any one of claims 51, 52, 57, and 58, wherein suppressing growth or activity of at least one bacterial species in the microbiota results in an increase in expression of one or more genes selected from the group consisting of Saa1, G6pc, Edn2, Bhmt, Gzma, Tnfsf10, Saa2, Tppp, Dio1, Tifa, Irf1, Cd38, Cc120, Socs2, Clec2d, Cc128, and Il17rd, in the intestinal and/or oropharyngeal and/or vaginal epithelium and lamina propria of said mammal.

89. The method of any one of claims 1-4, 24-27, 56, and 70, wherein
(i) Th17-specific cytokine, chemokine, or effector is selected from the group consisting of IL-17A, IL-17F, IL-21, IL-22, IL-23, CCL20, beta defensin 4, CD-161, and CCR6; defensin β1, Reg3γ, and Relmβ, and/or
(ii) Th2-specific cytokine is selected from the group consisting of IL-4, IL-5, IL-9, and IL-13, and/or
(iii) Treg-specific cytokine is selected from the group consisting of TGFβ, IL-10, and IL-35.

90. The method of any one of claims 13-16, 31-34, 57, 58, 71, and 72, wherein the SAA gene is selected from SAA1, SAA2, and SAA3.

91. The method of claim 25 or 27, wherein said bacterial strain is from a family selected from the group consisting of Clostridiaceae, Rikenellaceae, S24-7 (order Bacteroidales), Candidatus Arthromitus (segmented filamentous bacteria), Bacteroidaceae, Bifidobacteriaceae, Enterobacteriaceae, and Coriobacteriaceae.

92. The method of claim 25 or 27, wherein said bacterial strain is from a genus selected from the group consisting of SMB53 (family Clostridiaceae), Turicibacter, Lactobacillus, Roseburia, Ruminococcus, Dorea, Allobaculum, Candidatus Arthromitus (segmented filamentous bacteria), Bacteroides, Blautia, Bifidobacterium, and Klebsiella.

93. The method of claim 25 or 27, wherein said bacterial strain is from a species selected from the group consisting of Bacteroides uniformis, Blautia producta, Bifidobacterium pseudolongum, and Lactobacillus reuteri.

94. The method of any one of claims 1-93, wherein the mammal is human.

95. A method for determining a risk for developing type 1 diabetes in a subject, said method comprising:
(a) determining a relative abundance of one or more Bifidobacterium species in a gastrointestinal microbiota sample obtained from the subject;
(b) comparing the one or more relative abundances determined in step (a) to a healthy control relative abundance for the same species, and
(c) (i) determining that the subject is at high risk for developing type 1 diabetes if the relative abundance of the one or more Bifidobacterium species in the gastrointestinal microbiota sample from the subject is decreased at least two-fold as compared to the control, or (ii) determining that the subject is not at high risk for developing type 1 diabetes if the relative abundance of the one or more Bifidobacterium species in the gastrointestinal microbiota sample from the subject is not decreased as compared to the control.

96. The method of claim 95, wherein the gastrointestinal microbiota sample is selected from the group consisting of fecal, cecal, and ileal sample.

97. The method of claim 95, wherein the gastrointestinal microbiota sample is a fecal sample.

98. The method of claim 95, further comprising obtaining the gastrointestinal microbiota sample from the subject prior to step (a).

99. The method of claim 98, further comprising subjecting the gastrointestinal microbiota sample to a treatment to maintain DNA integrity.

100. The method of claim 99, wherein the treatment to maintain DNA integrity involves adding a preservative or freezing of the sample.

101. The method of claim 95, wherein the gastrointestinal microbiota sample had been subjected to a treatment to maintain DNA integrity.

102. The method of claim 101, wherein the treatment to maintain DNA integrity involves adding a preservative or freezing of the sample.

103. The method of claim 95, wherein the subject has not been exposed to antibiotics for at least one month before the microbiota sample is obtained.

104. The method of claim 95, wherein determining the relative abundance of the bacterial species comprises a method selected from the group consisting of quantitative polymerase chain reaction (qPCR), sequencing of bacterial 16S rRNA, and shotgun metagenome sequencing.

105. The method of claim 95, wherein the healthy control relative abundance is a predetermined standard.

106. The method of claim 95, wherein the healthy control relative abundance is obtained using a healthy subject or several healthy subjects of the same gender, age and ethnicity as the subject who is being diagnosed for type 1 diabetes.

107. The method of claim 106, wherein the healthy subject has not been exposed to antibiotics for at least one month before the microbiota sample is obtained.

108. The method of claim 95, further comprising treating the subject who has been determined to be at high risk for developing type 1 diabetes with a diabetes treatment.

109. The method of claim 108, wherein the diabetes treatment comprises administering to the subject a therapeutically effective amount of a compound or composition, wherein said compound or composition increases the abundance of at least one Bifidobacterium species in the gastrointestinal microbiota of the subject.

110. The method of claim 109, wherein said compound is a prebiotic.

111. The method of claim 109, wherein said composition comprises a probiotic and/or a prebiotic.

112. The method of claim 110 or claim 111, wherein the prebiotic is selected from the group consisting of a fructooligosaccharide (FOS), inulin, a galactooligosaccharide (GOS), a human milk oligosaccharide (HMO), Lacto-N-neotetraose, D-Tagatose, xylo-oligosaccharide (XOS), an arabinoxylan-oligosaccharide (AXOS), and any mixtures thereof.

113. The method of claim 111, wherein said probiotic is selected from the group consisting of live cells, conditionally lethal cells, spores, inactivated cells, killed cells, and a cell extract.

114. The method of claim 111, wherein said probiotic comprises at least one Bifidobacterium strain.

115. The method of claim 108, wherein the diabetes treatment comprises administering to the subject a therapeutically effective amount of a compound or composition, wherein said compound or composition decreases the abundance of Akkermansia mucinophilia in the gastrointestinal microbiota of the subject.

116. The method of claim 115, wherein said compound is a narrow spectrum antibiotic.

117. The method of claim 124, wherein said composition comprises a narrow spectrum antibiotic.

118. A method for preventing or delaying onset or decreasing severity of type 1 diabetes in a subject in need thereof, said method comprising administering to the subject a therapeutically effective amount of a compound or composition, wherein said compound or composition increases the abundance of at least one Bifidobacterium species in the gastrointestinal microbiota of the subject.

119. The method of claim 118, wherein the gastrointestinal microbiota is selected from the group consisting of fecal, cecal, and ileal microbiota.

120. The method of claim 118, wherein said compound is a prebiotic.

121. The method of claim 118, wherein said composition comprises a probiotic and/or a prebiotic.

122. The method of claim 120 or claim 121, wherein the prebiotic is selected from the group consisting of a fructooligosaccharide (FOS), inulin, a galactooligosaccharide (GOS), a human milk oligosaccharide (HMO), Lacto-N-neotetraose, D-Tagatose, a xylo-oligosaccharide (XOS), an arabinoxylan-oligosaccharide (AXOS), and any mixtures thereof.

123. The method of claim 121, wherein said probiotic is selected from the group consisting of live cells, conditionally lethal cells, spores, inactivated cells, killed cells, and a cell extract.

124. The method of claim 121, wherein said probiotic comprises at least one Bifidobacterium strain.

125. The method of claim 121, wherein the probiotic composition further comprises a buffering agent.

126. The method of claim 125, wherein the buffering agent is selected from the group consisting of sodium bicarbonate, dairy drinks, and infant formula.

127. The method of claim 126, wherein the dairy drinks are selected from the group consisting of milk, yougurt and kefir.

128. The method of claim 121, wherein the probiotic composition is administered conjointly with a prebiotic which stimulates growth and/or metabolic activity of bacteria contained in the probiotic composition.

129. The method of claim 128, wherein the probiotic and prebiotic are administered in one composition, or simultaneously as two separate compositions, or sequentially.

130. The method of claim 118, wherein said compound is a narrow spectrum antibiotic, which inhibits growth of one or more suppressors or competitors of Bifidobacterium.

131. The method of claim 118, wherein said composition comprises a narrow spectrum antibiotic, which inhibits growth of one or more suppressors or competitors of Bifidobacterium.

132. The method of claim 118, wherein the compound or composition is administered by a route selected from the group consisting of orally, rectally, fecally, and via naso/oro-gastric gavage.

133. The method of claim 118, wherein said compound or composition is contained within an infant formula.

134. The method of claim 118, wherein said compound or composition is administered to a pregnant woman who is in active labor.

135. The method of claim 134, wherein said compound or composition is administered to the vaginal area of the woman.

136. The method of claim 118, wherein said compound or composition is administered to a newborn child.

137. The method of claim 136, wherein said compound or composition is administered to the child's mouth and/or skin.

138. The method of claim 136, wherein said child was born via a C-section.

139. The method of claim 118, wherein said compound or composition is applied to mother's nipples during breastfeeding.

140. A method for enhancing a mucosal IgA response in a subject in need thereof, said method comprising administering to the subject a therapeutically effective amount of a compound or composition, wherein said compound or composition increases the abundance of at least one Bifidobacterium species in a mucosal microbiota of the subject.

141. A method for enhancing SAA1 gene expression and/or intestinal barrier function in a subject in need thereof, said method comprising administering to the subject a therapeutically effective amount of a compound or composition, wherein said compound or composition increases the abundance of at least one Bifidobacterium species in a mucosal microbiota of the subject.

142. A method for enhancing interferon-gamma (IFNγ) production in the spleen in a subject in need thereof, said method comprising administering to the subject a therapeutically effective amount of a compound or composition, wherein said compound or composition increases the abundance of at least one Bifidobacterium species in a mucosal microbiota of the subject.

143. A method for preventing or delaying onset or decreasing severity of a disease selected from the group consisting of Celiac disease, Graves disease, and Hashimoto's thyroiditis in a subject in need thereof, said method comprising administering to the subject a therapeutically effective amount of a compound or composition, wherein said compound or composition increases the abundance of at least one Bifidobacterium species in the gastrointestinal microbiota of the subject.

144. A method for preventing or delaying onset or decreasing severity of an early life male-dominated disease in a subject in need thereof, said method comprising administering to the subject a therapeutically effective amount of a compound or composition, wherein said compound or composition increases the abundance of at least one Bifidobacterium species in the gastrointestinal microbiota of the subject.

145. The method of claim 144, wherein the early life male-dominated disease is autism or attention-deficit disorder (ADD).

146. The method of any one of claims 140-144, wherein said compound is a prebiotic.

147. The method of any one of claims 140-144, wherein said composition comprises a probiotic and/or a prebiotic.

148. The method of claim 146 or claim 147, wherein the prebiotic is selected from the group consisting of a fructooligosaccharide (FOS), inulin, a galactooligosaccharide (GOS), a human milk oligosaccharide (HMO), Lacto-N-neotetraose, D-Tagatose, a xylo-oligosaccharide (XOS), an arabinoxylan-oligosaccharide (AXOS), and any mixtures thereof.

149. The method of claim 147, wherein said probiotic is selected from the group consisting of live cells, conditionally lethal cells, spores, inactivated cells, killed cells, and a cell extract.

150. The method of claim 147, wherein said probiotic comprises at least one Bifidobacterium strain.

151. The method of claim 147, wherein the probiotic composition further comprises a buffering agent.

152. The method of claim 151, wherein the buffering agent is selected from the group consisting of sodium bicarbonate, dairy drinks, and infant formula.

153. The method of claim 152, wherein the dairy drinks are selected from the group consisting of milk, yougurt and kefir.

154. The method of claim 147, wherein the probiotic composition is administered conjointly with a prebiotic which stimulates growth and/or metabolic activity of bacteria contained in the probiotic composition.

155. The method of claim 154, wherein the probiotic and prebiotic are administered in one composition, or simultaneously as two separate compositions, or sequentially.

156. The method of any one of claims 140-144, wherein said compound is a narrow spectrum antibiotic, which inhibits growth of one or more suppressors or competitors of Bifidobacterium.

157. The method of any one of claims 140-144, wherein said composition comprises a narrow spectrum antibiotic, which inhibits growth of one or more suppressors or competitors of Bifidobacterium.

158. The method of any one of claims 140-144, wherein the compound or composition is administered by a route selected from the group consisting of orally, rectally, fecally, and via naso/oro-gastric gavage.

159. The method of any one of claims 140-142, wherein the mucosal microbiota is a gastrointestinal microbiotia.

160. The method of any one of claims 140-144, wherein said compound or composition is contained within an infant formula.

161. The method of any one of claims 140-144, wherein said compound or composition is administered to a pregnant woman who is in active labor.

162. The method of claim 161, wherein said compound or composition is administered to the vaginal area of the woman.

163. The method of any one of claims 140-144, wherein said compound or composition is administered to a newborn child.

164. The method of claim 163, wherein said compound or composition is administered to the child's mouth and/or skin.

165. The method of claim 163 wherein said child was born via a C-section.

166. The method of any one of claims 140-144, wherein said compound or composition is applied to mother's nipples during breastfeeding.

167. The method of any one of claims 95-166, wherein the subject is human.

168. The method of claim 167, wherein the subject is a child.

169. The method of claim 118, wherein the subject is at high risk of developing type 1 diabetes.
